(19) United States
(12) Patent Application Publication
(10) Pub. No.: US 2023/0272002 A1
(43) Pub. Date: Aug. 31, 2023

(54) PHOTOREDOX PROTEIN MODIFICATION

(71) Applicants: THE ROSALIND FRANKLIN INSTITUTE, Didcot, Oxfordshire (GB); OXFORD UNIVERSITY INNOVATION LIMITED, Oxford (GB)

(72) Inventors: Patrick G. Isenegger, Oxford, Oxfordshire (GB); Benjamin G. Davis, Didcot, Oxfordshire (GB); Brian Josephson, Oxford, Oxfordshire (GB); Veronique Gouverneur, Oxford, Oxfordshire (GB); Charlie Fehl, Oxford, Oxfordshire (GB); Adeline W J Poh, Oxford, Oxfordshire (GB); Andrew M. Giltrap, Didcot, Oxfordshire (GB); Simon Nadal, Oxford, Oxfordshire (GB); Yibo Zeng, Didcot, Oxfordshire (GB); Jeroen Sap, Oxford, Oxfordshire (GB); Oluwatobi Arisa, Oxford, Oxfordshire (GB)

(21) Appl. No.: 18/005,519

(22) PCT Filed: Jul. 15, 2021
(86) PCT No.: PCT/GB2021/051826

§ 371 Date: Jan. 13, 2023

(30) Foreign Application Priority Data
Jul. 16, 2020 (GB) 2010989.8

(51) Int. Cl. C07K 1/107; C07D 213/71; C07F 5/02; C07D 277/76

(52) U.S. Cl. CPC C07K 1/1077; C07D 213/71; C07F 5/025; C07D 277/76

(57) ABSTRACT

The present invention relates to the photoredox-mediated functionalization of proteins with chemical groups via radical generated C—C bond formation, by using specific boronate and sulfone precursor compounds. The present invention also relates to functionalized proteins that can be generated via this method and to the specific boronate and sulfone precursor compounds themselves.

### FIELD OF THE INVENTION

The present invention relates to the photoredox-mediated functionalization of proteins with chemical groups via radical generated C—C bond formation, by using specific boronate and sulfone precursor compounds. The present invention also relates to functionalized proteins that can be generated via this method and to the specific boronate and sulfone precursor compounds themselves.

### BACKGROUND TO THE INVENTION

Post-translational modifications (PTMs) greatly expand the structures and functions of proteins in nature. The emergence of parallel, synthetic protein functionalization strategies now allows not only their direct mimicry, but also unnatural protein variants with diverse potential functions ranging from drug carrying, to tracking, imaging, and partner crosslinking. However, the range of functional groups that can be introduced by these modifications is still limited, especially for reactive functional groups.

Methods that use the translational machinery of the cell provide some advantages for installing select modifications into proteins, but can be limited in scope and efficiency. Fed unnatural amino acid precursors can be degraded or may not be tolerated during biosynthesis; this is especially the case for those with reactive side-chains. Post-translational functionalization offers an alternative strategy that, through its late-stage use, could be potentially broader in scope. In principle, it is only limited by the compatibility of the reaction conditions used with the protein substrate and its context.

In one version of post-translational functionalization, a readily-generated dehydroalanine (Dha) residue is used in proteins as a singly-occupied molecular orbital (SOMO) acceptor (‘radical acceptor’ or ‘SOMO-phile’) that is highly reactive towards several carbon radical species thereby allowing selective β, γ-C—C bond formation to introduce new side-chains in a ‘scarless/traceless’ manner. However, incompatibilities of side-chain/carbon radical precursors and the reagents that generate them (e.g. single electron transfer (SET) from metals or BH₄⁻) currently limit the scope of such techniques. Nonetheless, such homolytic 1e-chemistry has potential advantages over typical heterolytic 2e-reagents. The intrinsic challenges of biomolecule modification include: water-compatibility; requirement for a ‘benignness’; and low (or non-) reactivity towards a plethora of biogenic acids, amines, alcohols, and thiols (ready 2e-reactants) present in most biological environments. By contrast, water and native proteins are less reactive to most carbon radicals. Suitably placed SOMOphiles such as Dha can therefore allow more general chemo- and site-selectivity in certain 1e-chemistries.

Other methods for SET (and hence carbon radical initiation, either oxidative or reductive) exist. Catalytic protein methods bring clear advantages over prior super-stoichiometric methods, which can drive unwanted side-reactions. Furthermore, if regulated by a relatively benign, potentially tissue-penetrating, trigger such as light, it could allow additional layers of e.g. temporal, spatial and even kinetic control to complement those of 1e-chemo-selectivity. Light-stimulated outer-sphere electron transfer (ET) has seen a resurgence in applications to small molecules. However, its use in site-selective, biomolecule modification has been more limited. Leading examples have largely been restricted to peptides sometimes requiring mixed organic solvents and/or ET systems that sit towards the extremes of redox ‘windows’ and resulting side-reactions have been noted. Moreover, dependence on certain precursor moieties, such as α-C-carboxyl or β-C—H, that cannot be re-/pre-positioned, can limit the site of reaction and/or lead to lower site-selectivity due to abundance. These methods have therefore yet to reach their full potential in protein chemistry.

There is a need for a method of functionalizing proteins with post translational modifications in a manner which can be done selectively, reliably, and under benign, moderate redox potentials, and which allows the addition of reactive functional side chains.

### SUMMARY OF THE INVENTION

The present inventors have surprisingly discovered that using specific radical precursors such as boronic acid catechol-ester derivatives and aryl sulfonyl fluorine derivatives, in the presence of a photocatalyst, allows for the radical driven C—C bond formation between functional side chains and SOMO acceptor residues on a protein or peptide. This C—C sidechain-alteration within intact proteins allows native, chemical, post-translational modification of proteins or peptides.

The methods discovered by the present inventors allow for light driven electron transfer, to generate sidechain carbon radical-precursors, that allows C—C bond-formation without the need for harsh reaction conditions and organic solvents. Further, control of reaction redox allows site-selective modification with good conversions and minimal damage to the protein or peptide. Specifically, the inventors have discovered that the in situ generation of easily-oxidized boronic acid catechol-ester (BACED) derivatives generates RH₂C· radicals that can form native (βCH2-γCH2) linkages of natural residues and PTMs, whereas in situ potentiation of aryl sulfone fluoride deriviatives and specific bromo fluoride derivatives by Fe(II) can generate RFXC· radicals, such as RF₂C·, that form equivalent (βCH₂-γCXF) linkages bearing H→F-labels.

These reaction methods of the present invention can be performed quickly and with small amounts of reagents. Further, these reactions are chemically-tolerant, allowing for incorporation an unprecedented range of functional groups into diverse protein scaffolds and sites. Initiation can be applied chemoselectively in the presence of sensitive groups in C radical precursors, enabling installation of previously incompatible sidechains. This provides access to new function and reactivity in proteins. The novel methods described herein and proteins/peptides produced by them may find application in a number of areas, for example (a) to install radical precursors for homolytic on-protein radical-generation; (b) to study enzyme function with natural, unnatural, and ‘zero-size’-labeled post-translationally modified protein substrates via simultaneous sensing of both chemo- and stereo-selectivity; and (c) to create access to generalized ‘alkylator-proteins’ with a spectrum of heterolytic covalent-bond-forming activity (reacting diversely with small molecules at one extreme or selectively with protein targets through good mimicry at the other). The resulting post-translational access to new reactions and chemical groups on proteins is therefore useful in revealing and creating protein function.

Thus, the present inventors have demonstrated that a three-fold combination of: (i) electron transfer at benign, moderate redox potentials using (ii) side-chain functionalized C· radical precursors ‘redox-matched’ with low, even substoichiometric, amounts of photocatalyst, triggered by (iii) light of appropriate flux, allows the generation and use of both off-protein and on-protein radicals to modify proteins via C—C bond formation (see FIG. 1). The resulting chemistry allows installation of unprecedented side-chains with new functional modes.

In a first embodiment the present invention provides a method of functionalizing a protein or peptide with a functional side chain moiety, wherein the protein or peptide comprises at least one singly occupied molecular orbital (SOMO) acceptor residue, wherein said SOMO acceptor is a residue comprising a side chain having an alkene group; wherein the method comprises:
(a) contacting the protein or peptide with a radical precursor compound and a photocatalyst having an oxidative half potential (Eₒₓ) of less than or equal to +1.2 V in its photo-activated state, when measured against a saturated calomel electrode, and
(b) exposing the resultant composition to light radiation in order to provide a functionalized protein or peptide;

wherein the radical precursor compound is selected from formula (II) or formula (III) below

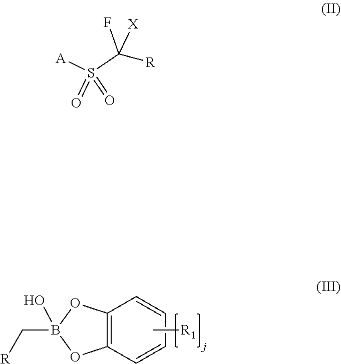

wherein R is the functional side chain moiety which is attached to the protein or peptide via the group —CFX— where the compound of formula (II) is used, or via the group —CH₂— where the compound of formula (III) is used;

X is selected from the group consisting of hydrogen, fluorine, chlorine, —C(O)OH, and —C(O)NH₂;

A is an aryl or heteroaryl group, which is optionally substituted by one or more R₂ groups;

j is 0, 1, 2, or 3;

R₁ and R₂ are independently selected from the group consisting of halogen and C₍₁₋₆₎ alkyl which is unsubstituted or substituted with one or more groups selected from hydroxy, oxy, halogen, amino, carboxy, C₍₁₋₆₎ ester, and C₍₁₋₆₎ ether; and wherein when a compound of formula (II) is used as the radical precursor, step (a) further comprises contacting the protein or peptide with a source of Fe(II).

In a further aspect of the method described above R is (i) a group selected from pharmaceutical drugs, sugars, polysaccharides, peptides, proteins, vaccines, antibodies, nucleic acids, viruses, labelling compounds, stabilized radical precursors, biomolecules and polymers, any of which may optionally be connected via a linker group.

In a further aspect of the methods described above the linker is a group L1 which is selected from alkyl in which one or more non-adjacent carbon atoms may be optionally substituted for a group selected from NH, O, S, —C(O)NH— or —NHC(O)—; polyethyleneglycol and analogues thereof; saccharides; polysaccharides; polyglycine; polyamides; or combinations of two or more of these groups.

In a further aspect of the first embodiment above R is (ii) a functional group R^{F}; or one or more functional groups R^{F} connected via a linker group L2; wherein R^{F} is
hydrogen, C₃₋₁₀ cycloalkyl, aryl or heteroaryl; wherein the cycloalkyl, aryl and heteroaryl groups are unsubstituted or substituted by one or more groups selected from ═O, ═NRa, Y and (C₁₋₆ alkyl)-Y; or
a reactive group Y selected from C₂₋₆ alkenyl, C₂₋₆ alkynyl, halogen, hydroxy, —OR^{a}, —SR^{a}, —S(O)Ra, —S(O)₂R^{a}, —OSO₃R^{a}, —NR^{a}C(O)R^{b}, —NR^{a}CO₂R^{b}, —NHC(O)NR^{a}R^{b}, —NHCNH₂NR^{a}R^{b}, —NR^{a}SO₂R^{b}, —N(SO₂R^{a})₂, —NHSO₂NR^{a}R^{b}, —OC(O)R^{a}, —C(O)R^{a}, —CO₂R^{a}, —C(O)NR^{a}R^{b}, —C(O)(NHNH₂), —ONH₂, —C(O)N(OR^{a})R^{b}, —SO₂NR^{a}R^{b} or —SO(NR^{a})R^{b}; cyano, nitro, C₁₋₆ azidoalkyl, —NR^{a}R^{b} and —(NR^{a}R^{b}R^{c})+;

wherein:

R^{a}, R^{b}, and R^{c} independently in each instance represent hydrogen, C₁₋₆ alkyl, C₃₋₁₀ cycloalkyl, heterocyclyl, phenyl, benzyl and heteroaryl, wherein the alkyl, cycloalkyl, heterocyclyl, phenyl, benzyl and heteroaryl groups at R^{a}, R^{b}, and R^{c} are unsubstituted or substituted by one or more substituents selected from halogen, hydroxy, ═O, —NH₂, —SO₃⁻, and C₁₋₆ alkoxy; and

L2 is selected from alkyl in which one or more non-adjacent carbon atoms may be optionally substituted for a group selected from NH, O, S, —C(O)NH— or —NHC(O)—; polyethyleneglycol and analogues thereof; saccharides; polysaccharides; polyglycine; polyamides; or combinations of two or more of these groups.

In a further aspect R is (ii) a functional group R^{F}; or one or more functional groups R^{F} connected via a linker group L2, wherein R^{F} is a reactive moiety selected from: C₂₋₆ alkenyl, C₂₋₆ alkynyl, halogen, —OC(O)R^{a}, —C(O)R^{a}, —CO₂R^{a}, —C(O)(NHNH₂), —ONH₂ and C₁₋₆ azidoalkyl; or R contains a reactive moiety of formula

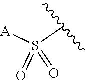

wherein A is as defined in claim 1; and wherein the reactive moiety

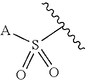

may optionally be connected via a linker group L2;

wherein L2 is an alkyl group in which one or more non-adjacent carbon atoms may be optionally substituted for a group selected from NH, O, S, —C(O)NH— or —NHC(O)—.

In a further aspect the reactive moiety is selected from halogen, C₁₋₆ azido, C₂₋₆ alkynyl,

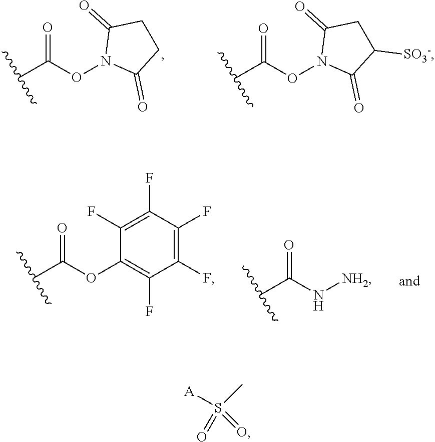

preferably

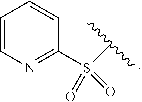 [SMILES: *S(=O)(=O)c1ccccn1]

In a second embodiment, the present invention provides a method of functionalizing a protein or peptide comprising at least one SOMO acceptor residue, as defined in the first embodiment above, with a functional side chain moiety, wherein the method comprises:
(a) contacting the protein or peptide with a radical precursor compound, a source of Fe(II) and a photocatalyst having an oxidative half potential (Eₒₓ) of less than or equal to +1.2 V in its photo-activated state when measured against a saturated calomel electrode; and
(b) exposing the resultant composition to light radiation in order to provide a functionalized protein or peptide;

wherein the radical precursor compound is a group of formula (IV) below,

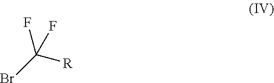

wherein R is the functional side chain moiety, which is attached to the protein or peptide via the group —CFX—; and wherein the group R is selected from —COOR^{d} and —CONR^{d}R^{e} wherein R^{d} represents hydrogen, C₁₋₆ alkyl, C₃₋₁₀ cycloalkyl, heterocyclyl, phenyl, benzyl or heteroaryl, wherein the alkyl, cycloalkyl, heterocyclyl, phenyl, benzyl, and heteroaryl groups at R^{d} are unsubstituted or substituted by one or more substituents selected from halogen, hydroxy, ═O, —NH₂, C₁₋₆ alkoxy and —NHCOR^{e}; and R^{e} represents hydrogen or C₁₋₄ alkyl.

In a third embodiment, the present invention provides a method of functionalizing a protein or peptide comprising at least one SOMO acceptor residue as defined in the first embodiment above with a functional side chain moiety having the structure

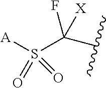

wherein the method comprises
(a) contacting the protein or peptide with a radical precursor compound, a source of Fe(II) and a photocatalyst having an oxidative half potential (Eₒₓ) of less than or equal to +1.2 V in its photo-activated state, when measured against a saturated calomel electrode; and
(b) exposing the resultant composition to light radiation in order to provide a functionalized protein or peptide;

wherein the radical precursor compound used has the following structure

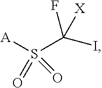

wherein the groups A and X are as defined in the first embodiment above.

In a further aspect of the above embodiments, when the functional side chain moiety comprises a reactive moiety as defined above, the method may further comprise reacting the peptide or protein via one of the reactive moieties to connect the functional side chain to a further molecule.

In a preferred aspect the further molecule is a pharmaceutical drug, a sugar, a polysaccharide, a peptide, a protein, a vaccine, an antibody, a nucleic acid, a virus, a labelling compound, a biomolecule or a polymer.

In a further aspect of any of the above embodiments, the SOMO acceptor residue is dehydroalanine.

In a further aspect of the above embodiments the group A is phenyl, pyridinyl, pyrimidinyl, benzothiazolyl or pyrazinyl.

In a preferred aspect of the above embodiments the group A is pyridinyl, pyrimidinyl or benzothiazolyl.

In a further preferred aspect of the above embodiments, the group A is 2-pyridinyl.

In a further aspect of the above embodiments the group X is fluorine.

In a further aspect of any of the above embodiments the source of Fe(II) is iron(II)sulfate, FeOTf₂, Fe(ClO₄)₂, FeF₂, or (NH₄)₂Fe(SO₄)₂, preferably FeSO₄·7H₂O.

In a further aspect of any of the above embodiments the photocatalyst is a Ru(II) or Ir(II) based catalyst, preferably a Ru(II) catalyst.

In a further preferred aspect of the above embodiments the Ru(II) photocatalyst is Ru(bpy)₃Cl₂ or Ru(bpm)₃Cl₂.

In a further aspect of any of the above embodiments the light radiation is in the region of 300 to 600 nm, preferably 400 to 500 nm, more preferably 430 to 470 nm.

In a further aspect of the above embodiments, when the radical precursor compound is a compound of formula (III), the compound of formula (III) is generated in situ by contacting the protein or polypeptide in step (a) with a functionalized boron compound comprising a —BCH₂R moiety, and a catechol derivative represented by the formula (IIIB) below:

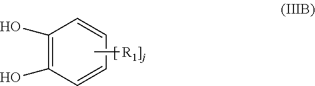

wherein R, R₁ and j are as defined in any above embodiments.

In a fourth embodiment the present invention provides a functionalized peptide or protein, comprising at least one residue of formula (IA):

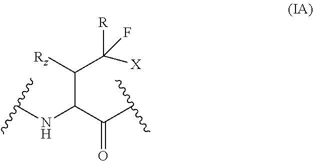

wherein X is selected from hydrogen, fluorine, —COOH, and —CONH₂, preferably fluorine; R_{Z} is hydrogen or methyl; and

R is as defined in any of the above embodiments.

In a further aspect of the above embodiment R is C₁₋₆ haloalkyl, C₁₋₆ azidoalkyl, or

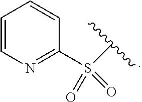 [SMILES: *S(=O)(=O)c1ccccn1]

In a further aspect of the above embodiment the residue of formula (IA) is any one of the compounds listed in examples 2a to 2ag.

In a further aspect of the above embodiment X is fluorine.

In a fifth embodiment the present invention provides a functionalized peptide or protein, comprising at least one residue of formula (IB).

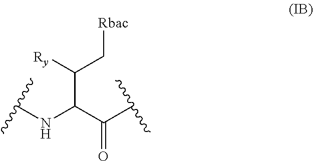

wherein Ry is hydrogen or methyl;

wherein Rbac is C₁₋₆ alkyl wherein the terminal carbon is substituted by at least one halogen, or Rbac is represented by the formula below

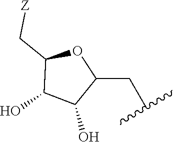

wherein Z is halogen.

In a sixth embodiment the present invention provides a method of covalently linking a functionalized protein or peptide according to the fourth or fifth embodiments described above with a further protein or peptide, wherein the group R or Rbac in the functionalized protein or peptide is C₁₋₆ haloalkyl, and wherein the further protein or peptide comprises a group capable of reacting with an alkyl halide to form a covalent bond.

In a further aspect of the above embodiment the functionalized protein or peptide is a substrate for the further protein or peptide, and the alkyl halide group is held in a binding pocket of the other protein or peptide in order to bring said alkylhalide group into proximity with the group capable of reacting with the alkylhalide group.

In a seventh embodiment, the present invention provides a method of covalently linking a functionalized protein or peptide according to the fourth embodiment with a further protein or peptide, wherein the group R in the functionalized protein or peptide is

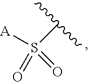

wherein the further protein or peptide comprises a group capable of reacting with a radical species to form a covalent bond, and wherein A is as defined in any of the above embodiments.

In an eighth embodiment, the present invention provides a compound according to formula (II) or (III) below:

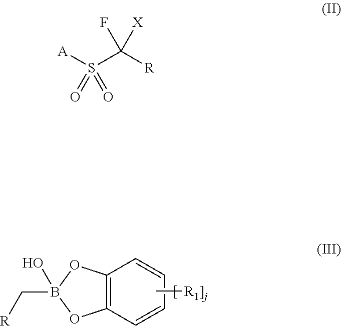

wherein A, X, R₁, and j are as defined in any of the above embodiments.

### BRIEF DESCRIPTION OF THE FIGURES

FIG. 1: On the left hand side is shown a schematic representation of the methods of the present invention, wherein BACED (left) and pySOOF (right) derivatives are reacted with a Dha containing residue to provide a functionalized protein. The top right shows some of the diverse range of protein scaffolds and sites which may be functionalized using the methods described herein. The bottom right shows some of the diverse range of functional groups which may be conjugated to proteins or peptides via the methods of the present invention.

FIG. 2(a) shows an oxidative half potential (Eₒₓ) spectrum for catalyst compatibility with protein-based chemistry at the top, including relevant catalysts found in literature (catalysts 1 to 5), as well as the oxidative half potentials of the BACED reagents, catechol and the boron precursor compounds.

FIG. 2(b) shows the voltammetric responses of 1 mM catechol and 12 mM phenethylboronic acid on GC in PBS, pH 7.10.

FIG. 2(c) shows a detailed reaction scheme for an example BACED reaction scheme according to embodiment (ii) described below, wherein a Dha residue is generated and functionalized with a specific side chain. Specifically, this scheme demonstrates the [Ru¹¹]-catalyzed, low-Eₒₓ activation (as compared to other derivatives) of the BACED reagent to RCH₂· radicals that then react with Dha to install side-chains in Histone H protein. Further, intact protein LC-MS (see right hand side chromatogram and m/z) shows homohomophenylalanine (1h) installation into Histone H3 protein.

FIG. 2(d) shows a detailed reaction scheme for an example pySOOF reaction scheme according to embodiment (i) described below, wherein a Dha residue is generated and functionalized with a specific side chain. Specifically, this scheme demonstrates [Ru¹¹]-catalyzed activation of pySOOF reagents to RCF2· radicals that then react with Dha in proteins to install ‘zero-size’-labelled side-chains. Added [Fe¹¹] drives unprecedented efficiency (2-5 equivalents of precursor) by suppressing oxidation by [Ru¹¹]* to imine (and hydrate) that suggests a key role as a reductant (readily-available in Biology) that quenches the alpha-C· radical adduct generated during the reaction. Intact protein LC-MS shows difluoroethylglycine (DfeGly, 2a) installation into Histone H3 protein is successful with [Fe¹¹] (see top right chromatogram and m/z), with improved conversion over the reaction without iron (see bottom center where unwanted side products were generated).

FIG. 3 shows a reaction scheme for on-protein homolytic and heterolytic reactivity via the installation of radical-precursor and electrophile side-chains.

FIG. 3(A) shows utilization of an iodo-functionalized pySOOF derivative according to embodiment (1a) of the method described below. This scheme shows the reductive installation of an on-protein pySOOF side-chain that is itself a protein radical precursor (as highlighted). Both mono- and difluoro-pySOOF sidechains could be installed via this method. The reagents and conditions used were: Histone H3-Dha9 (66 μM), Iodo-pySOOF (2 eq), FeSO₄·7H₂O (20 eq), Ru(bpy)₃Cl₂ (0.4 eq), NH₄OAc (500 mM, pH 6, 3 M GdnHCl), 50 W Blue LED, RT, 15 min. Intact protein LC-MS is shown in the bottom right boxed insert.

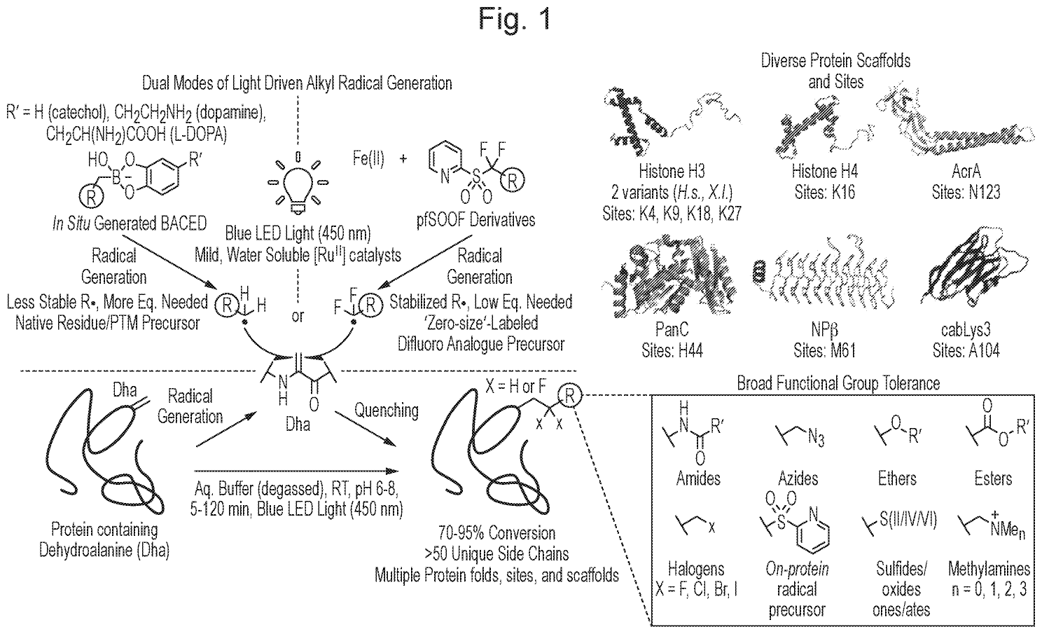

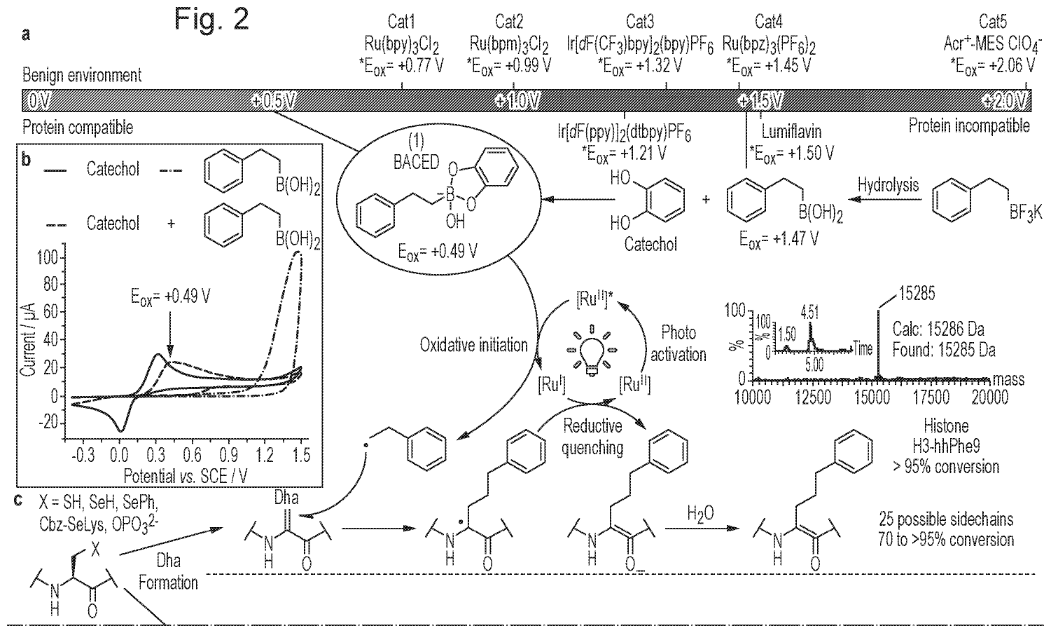

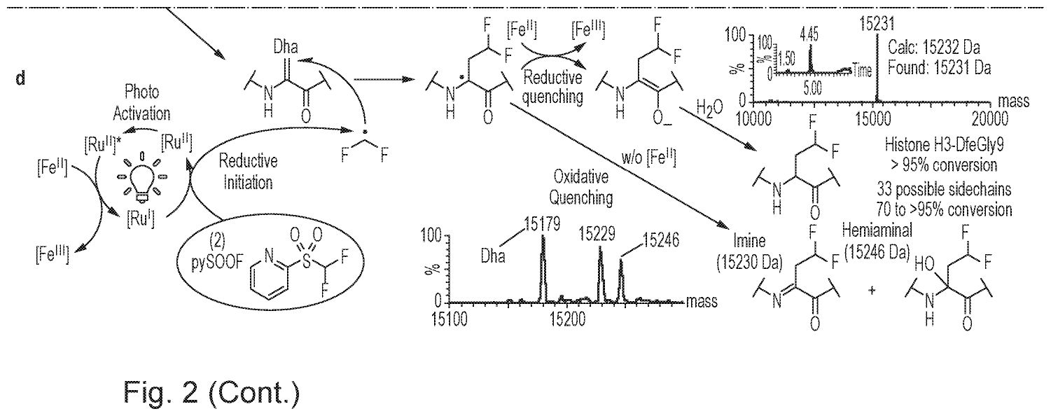

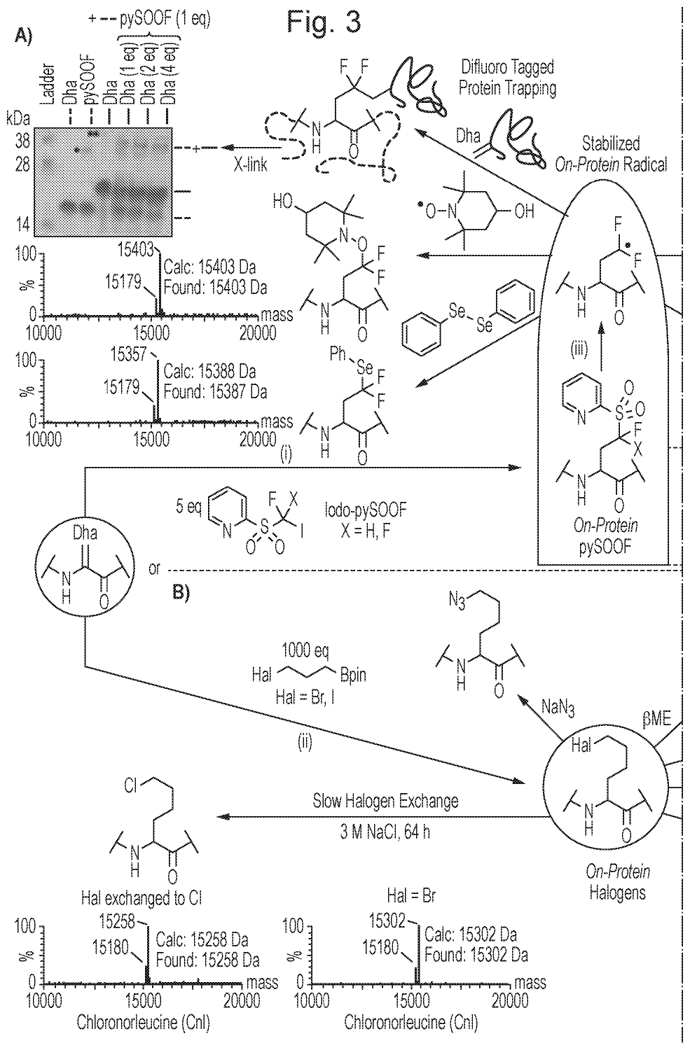

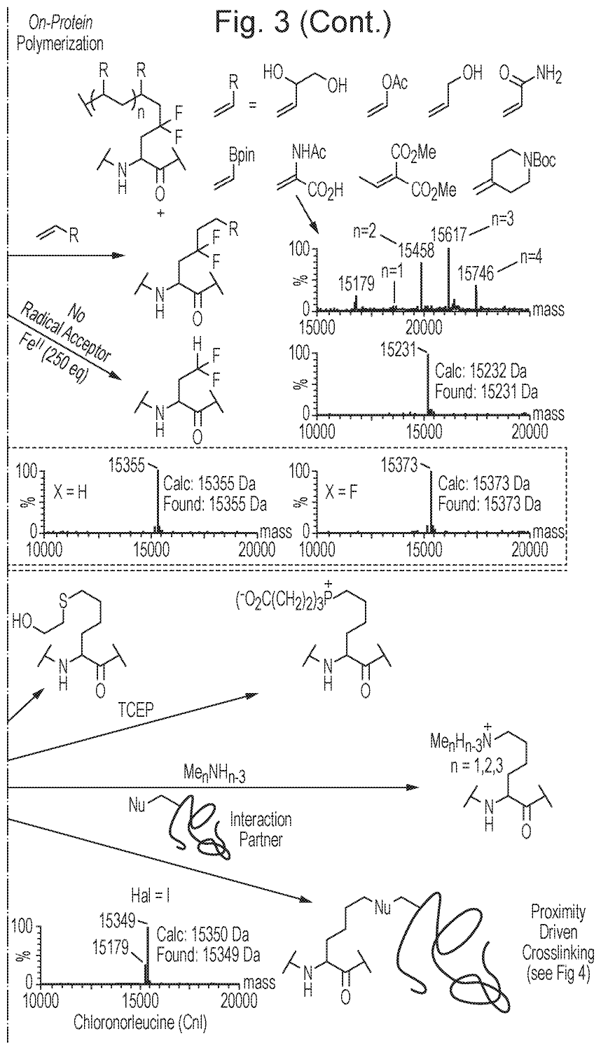

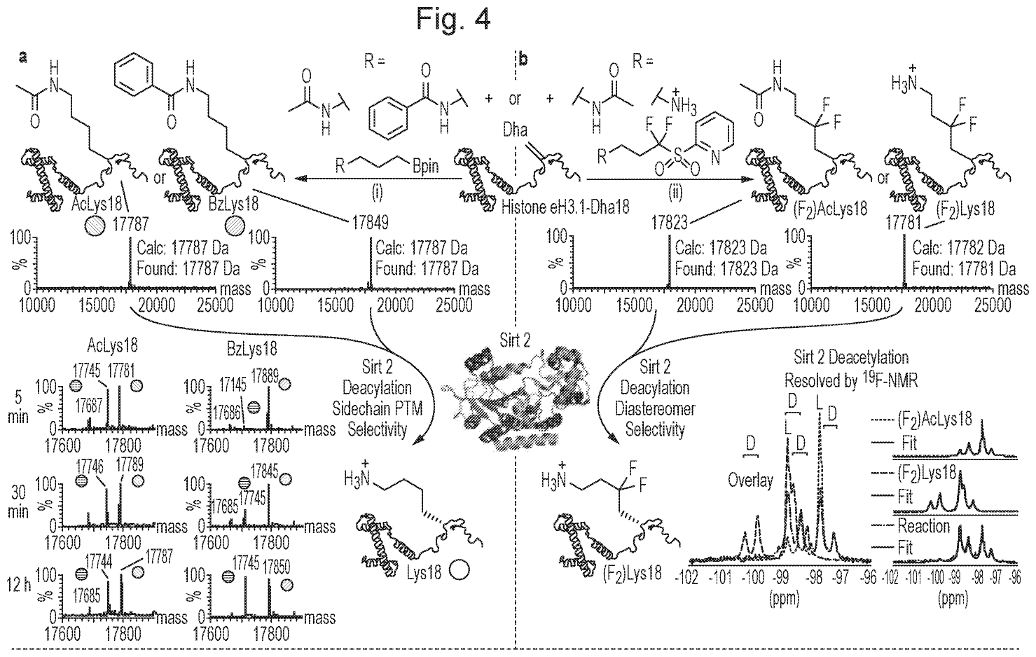

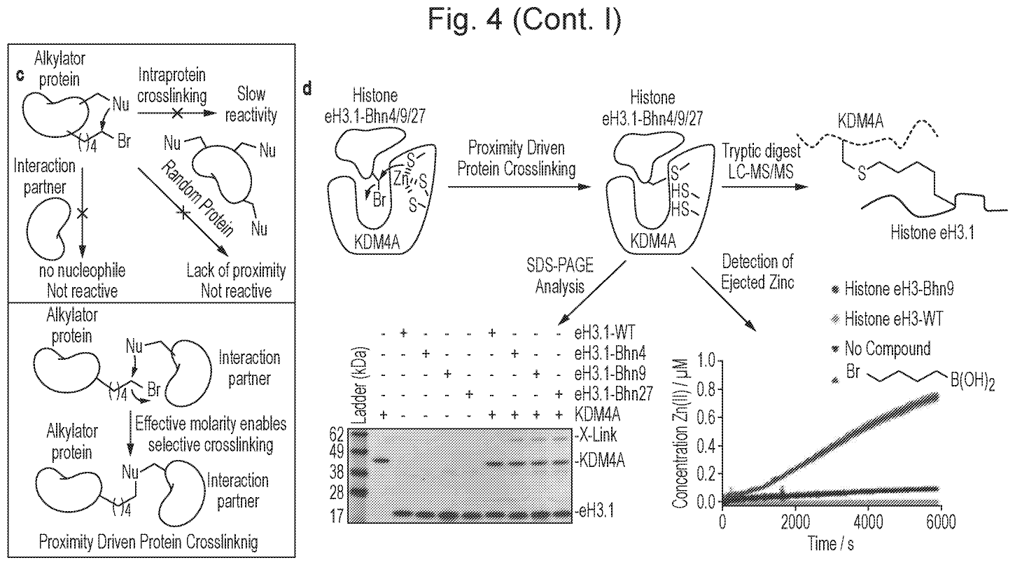

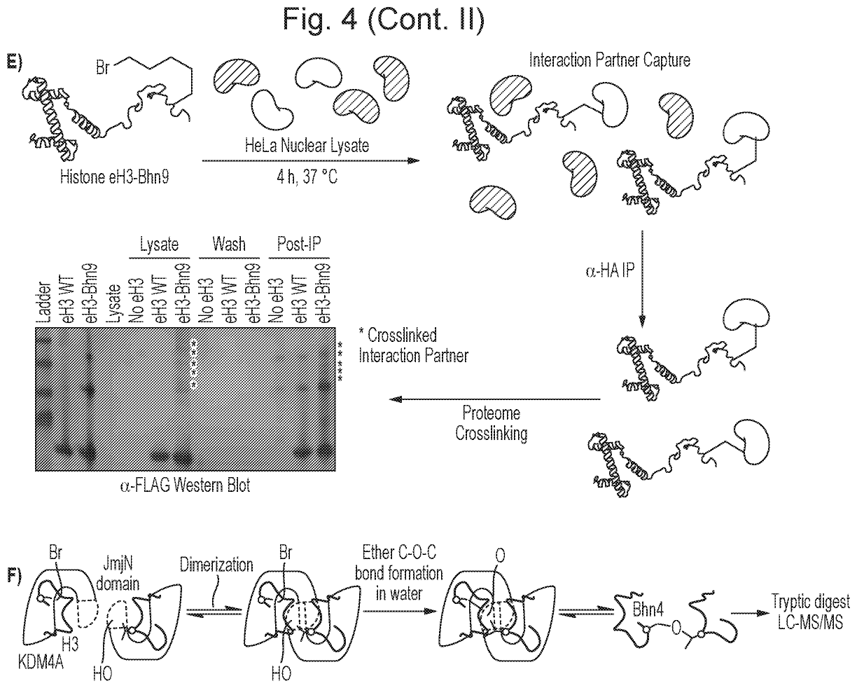

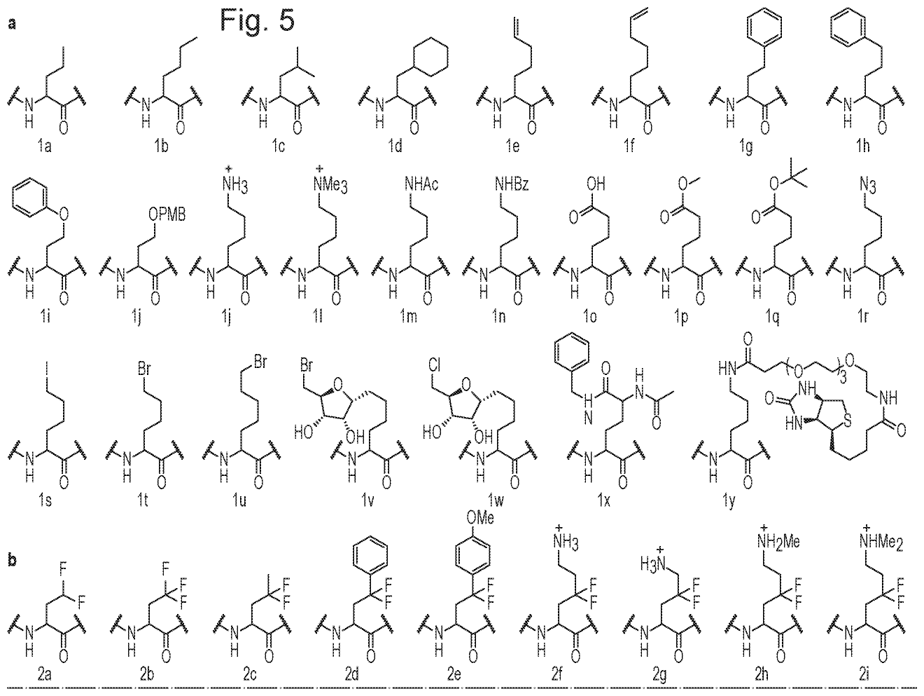

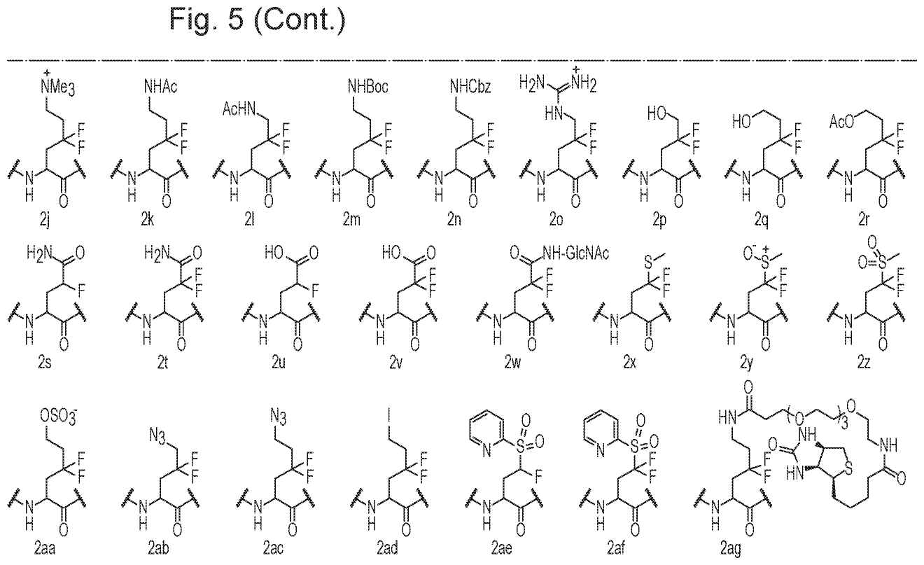

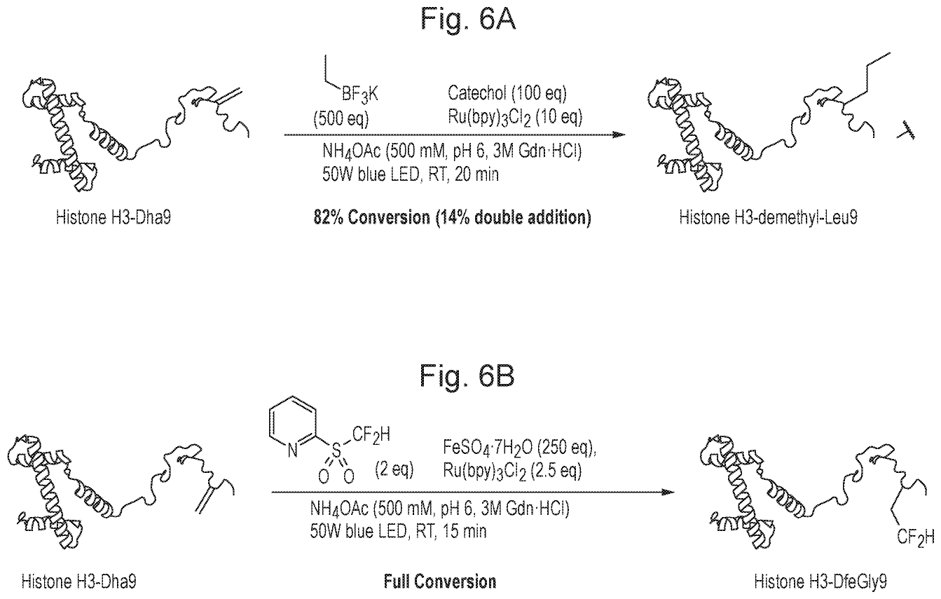

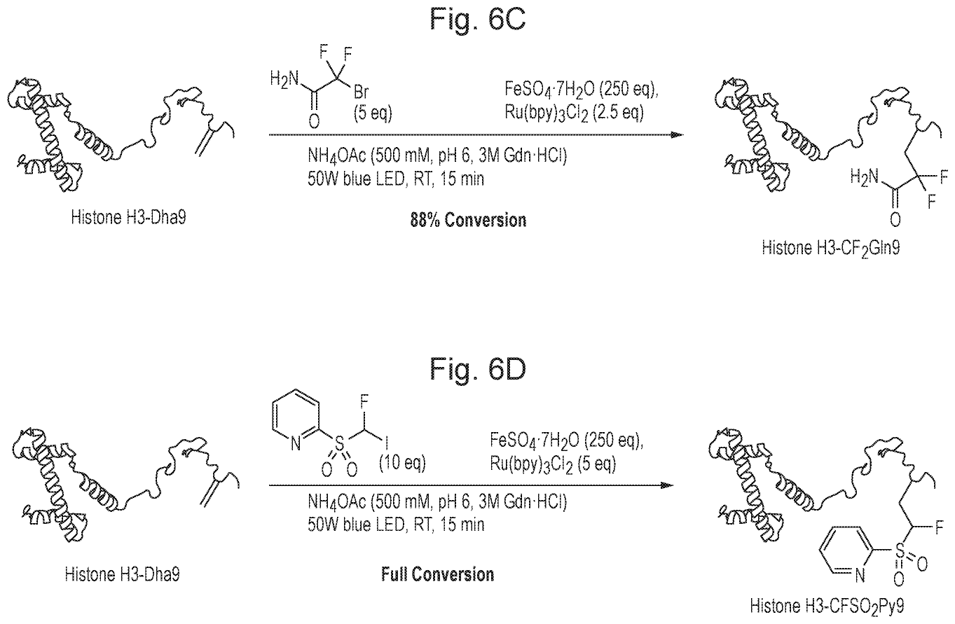

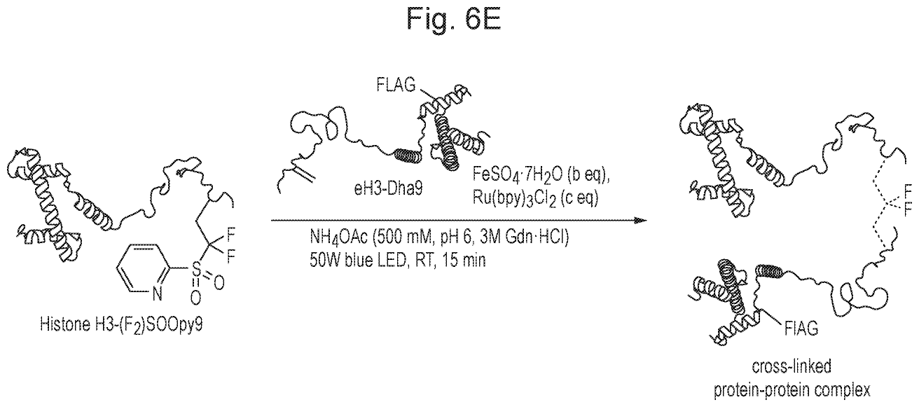

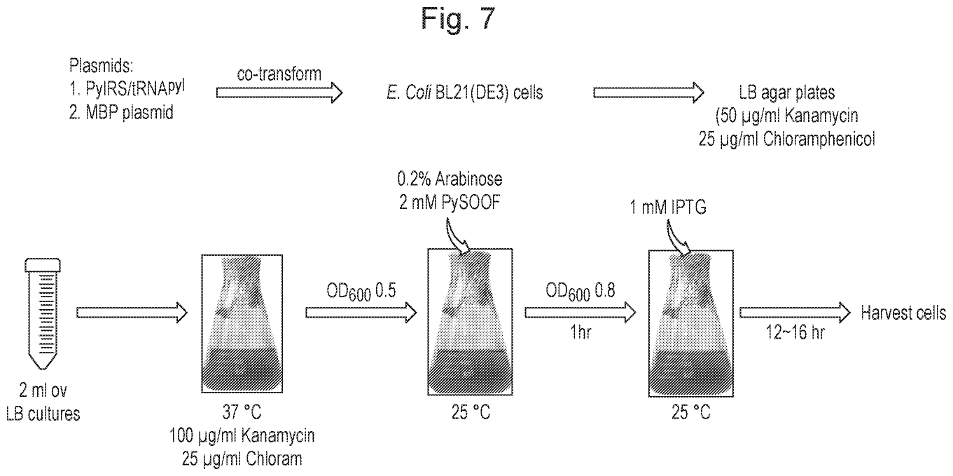

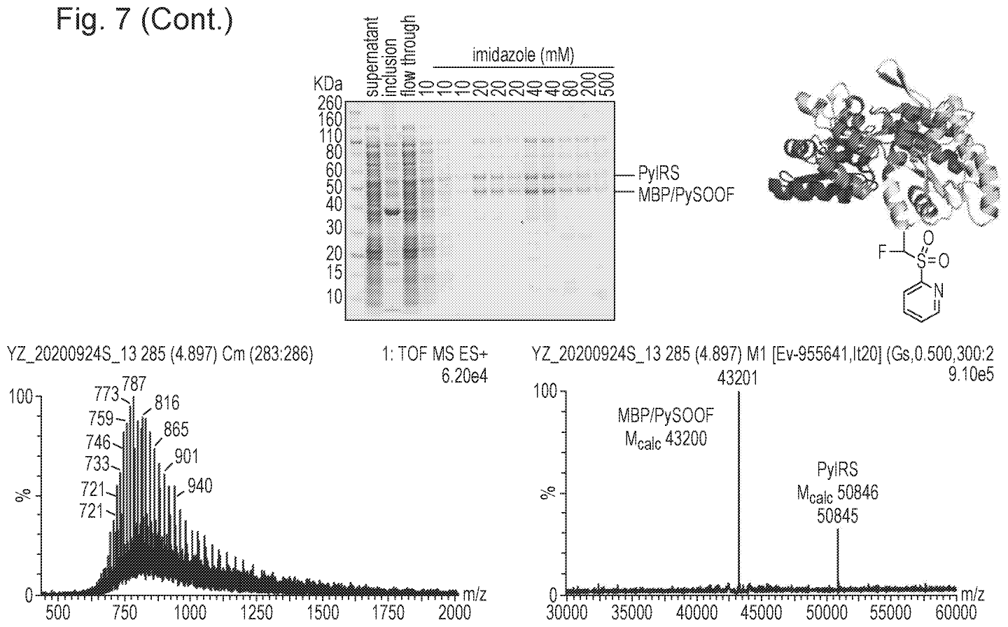

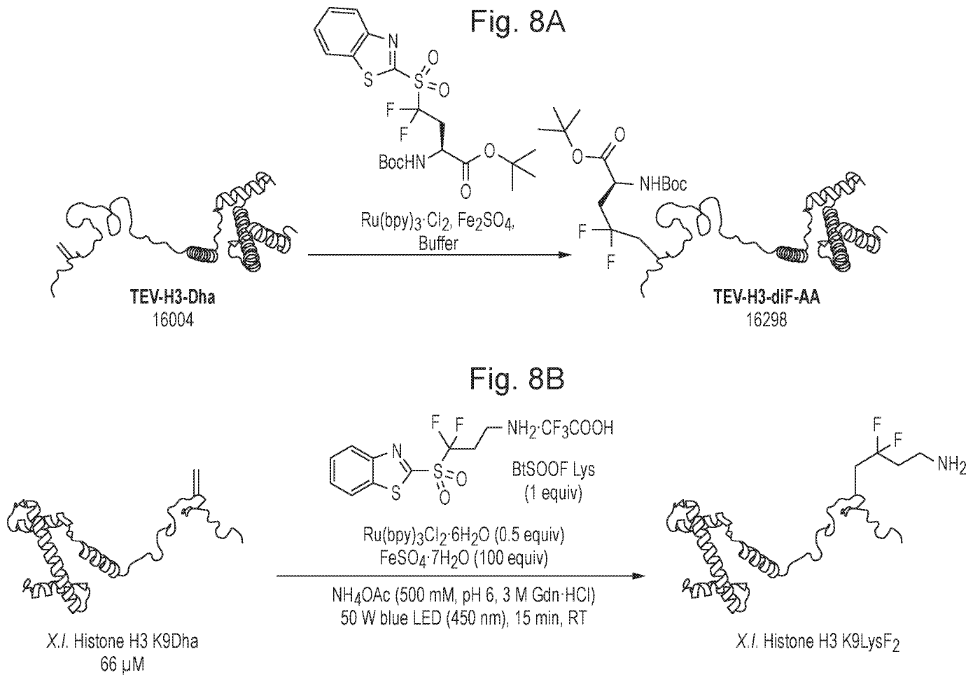

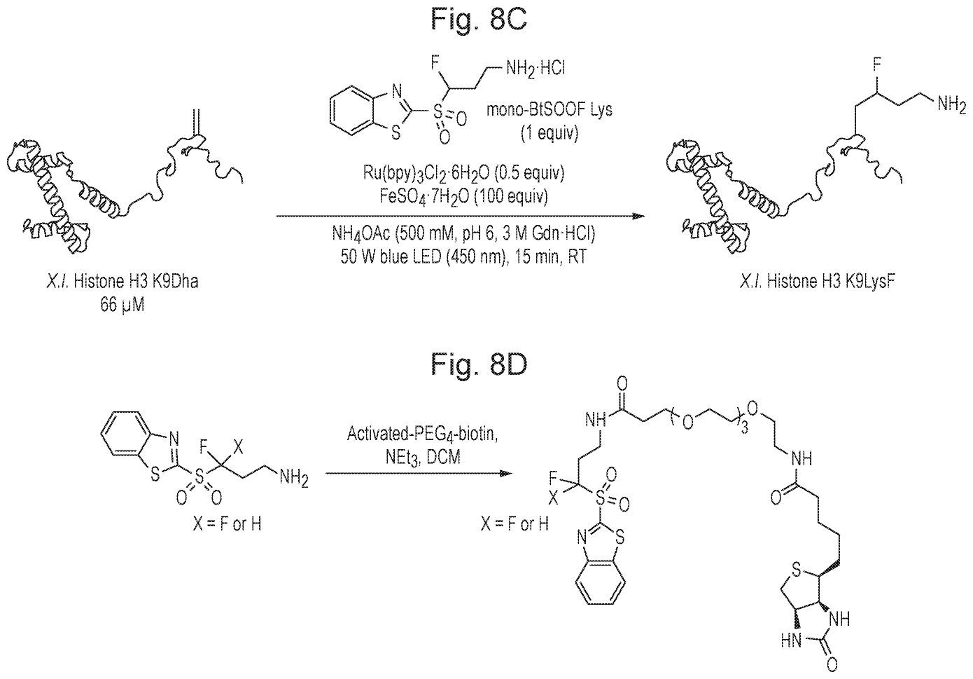

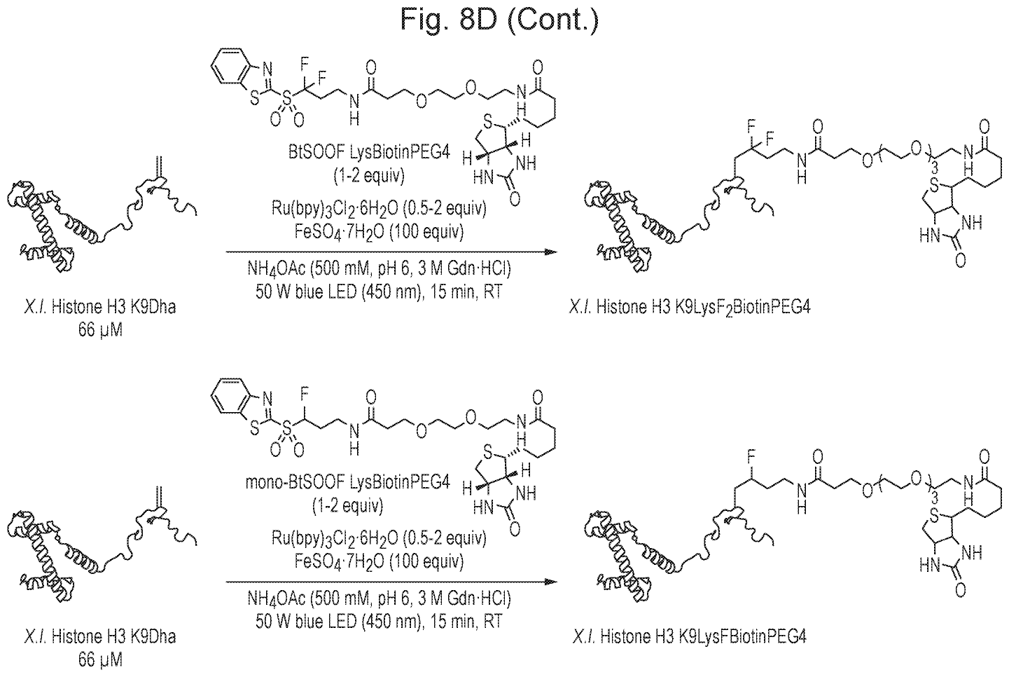

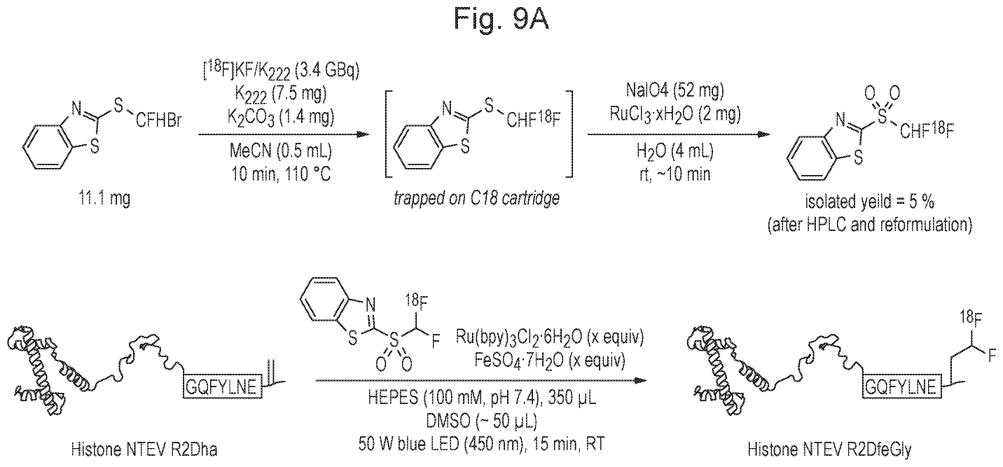

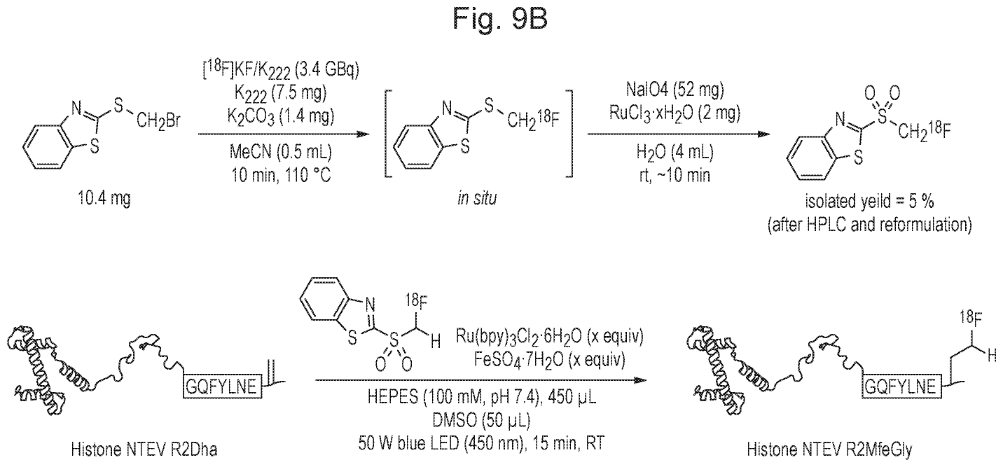

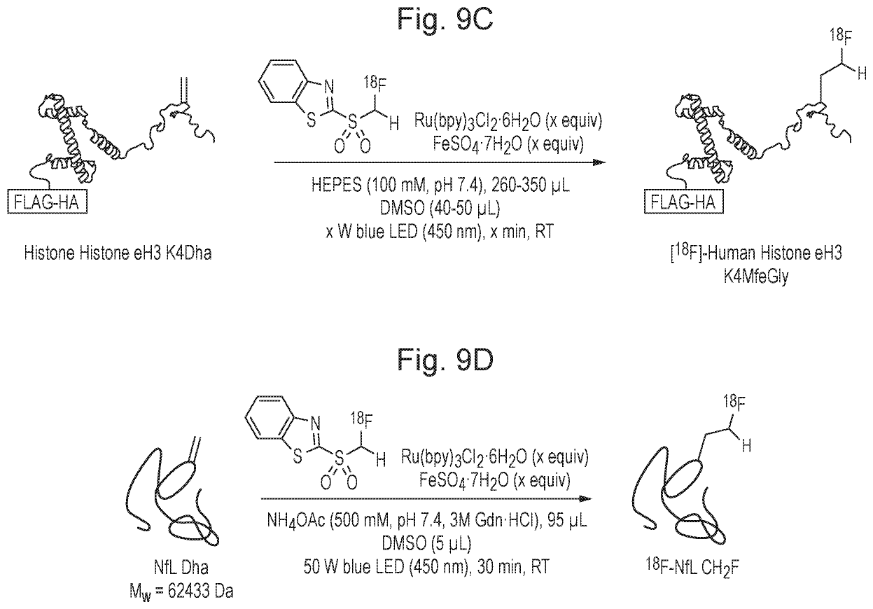

After activation using our standard, mild conditions (see FIG. 2), the resulting on-protein radical allowed diverse, further protein functionalization via various on-protein homolytic bond-forming modes. The on-protein radical could either be: polymerized with various radical acceptors via C—C-bond-formation (right, top); C—C-trapped with another Dha-containing protein to promote C—C-bond-forming protein-protein crosslinking (left, top), quenched with stable-O radical nitroxide radical TEMPO to form C—O bonds (left, middle); used to cleave diselenide (SePh)₂ to form C—Se bonds (left, bottom); or reduced (overall C—H bond-formation) to difluoroethylglycine (DfeGly) with additional Fe (right, middle). The reagents and conditions used were: Histone H3-pySOOF9 (66 μM), substrate (10-250 eq), FeSO₄·7H₂O (0-25 eq), Ru(bpy)₃Cl₂ (1-5 eq), NH₄OAc (500 mM, pH 6, 3 M GdnHCl), 50 W Blue LED, RT, 15 min, see Example 4 for reaction details, residual Dha=15179 Da]. FIG. 3(B) shows utilization of an alkylhalide-functionalized BACED according to embodiment (ii) of the method described below. The scheme shows oxidative installation that leaves the C-Halogen (C-Hal) bond unperturbed. This installs on-protein alkylhalide electrophile side-chains (highlighted). The reagents and conditions used were: Histone H3-Dha9 (66 μM), alkylboronic acid pinacol ester (1000 eq), catechol (100 eq), Ru(bpm)₃Cl₂ (10 eq), NH₄OAc (500 mM, pH 6, 3 M GdnHCl), 50 W Blue LED, RT, 1-3 h). This provided a further reaction platform for diverse, on-protein heterolytic bond-forming modes. These on-protein alkylhalide electrophiles could be reacted through substitution with various small molecule P, S, N and Hal nucleophiles (TCEP=tris(2-carboxyethyl)phosphine, PME=betamercaptoethanol), allowing diverse C—P, C—S, C—N, and C-Hal bonds (see Example 3 for details, residual Dha=15179 or 15180 Da) at higher concentrations. Furthermore, the ability to install a range of inherently-reactive alkylhalide side-chains in this way (e.g., chloro-(Cnl), bromo-(Bnl), iodo-(Inl) norleucines, see intact protein LC-MS, left, bottom) allowed proximity-driven protein-protein crosslinking with interaction partners (see FIG. 4).

FIG. 4 shows the specific editing insertion of native, difluoro-labeled, and electrophile-containing sidechains into proteins. Such modifications provide insight into enzymes that post-translationally modify proteins and can be used to bind other proteins or enzymes. For example, Sirt2 enzyme was shown to display different deacylation rates (as shown by intact-protein LC-MS monitoring) towards installed acetyl- and benzoyl-lysine on Histone eH3-K18 proteins. Deacetylation was also directly and site-specifically monitored via ¹⁹F-NMR via the difluoro-tag on the CγF₂ gamma carbon of installed Lys and AcLys sidechains. Although four-bonds-distant from site of PTM, CγF₂-labels display sufficient sensitivity to chemical environment (δF perturbation) to allow direct simultaneous monitoring of Sirt2's chemo- and stereo-selectivity during processing.

FIG. 4(A) shows the functionalization of Histone H3 with BACED reagent according to embodiment (ii)/(iia) of the methods described below for use in the above enzyme studies. The reagents and conditions used for installation were: Histone H3-Dha9 (66 μM), alkylboronic acid pinacol ester (250 eq), catechol (100 eq), Ru(bpm)₃Cl₂ (10 eq), NH₄OAc (500 mM, pH 6, 3 M GdnHCl), 50 W Blue LED, RT, 1 h.

FIG. 4(B) shows the functionalization of Histone H3 with pySOOF type reagent according to embodiment (i) of the methods described below for use in the above enzyme studies. The reagents and conditions used for installation were: Histone H3-Dha9 (66 μM), alkyl-pySOOF (50 eq), FeSO₄·7H₂O (50 eq), Ru(bpy)₃Cl₂ (2 eq), NH₄OAc (500 mM, pH 6, 3 M GdnHCl), 50 W Blue LED, RT, 15 min. Met ox=15838 Da.

FIG. 4(C) shows a general diagram for ideal traits of an ‘alkylator protein’: reactions to limit or avoid are shown in the upper box, and desired, selective reactions are shown in the bottom box.

FIG. 4(D) shows that crosslinking between KDM4A and Histone-eH3.1-Bhn4/9/27 (Bhn=bromohomonorleucine) traps KDM4A-Zn-binding-cysteines near active site. Coomassie-Blue-SDS-PAGE (bottom left), tryptic-LC-MS/MS (top right) and Zn(II)-ejection (bottom right) confirm crosslinking between KDM4A and Histone-eH3.1-Bhn9 (see also ED FIG. 10c) [Zn(II)-ejection rates: eH3-Bhn9=9.27±0.025 nM/min, eH3-WT=0.09+0.006 nM/min, 1u-precursor=0.805±0.010 nM/min, no-compound=0.87+0.028 nM/min, N=3 independent experiments. Data plotted is average+/−standard deviation (N=3 technical replicates), p<0.0001 1-way ANOVA]. See also ED FIG. 10 for further alkylator protein experiments.

FIG. 4(E) shows Histone eH3.1-Bhn9 alkylator protein was incubated with HeLa nuclear lysate to capture interaction partners via proximity-driven crosslinking. After an enrichment via the HA-tag (on Histone eH3.1), an α-FLAG western blot reveals multiple higher MW bands corresponding to the mass of the histone plus that of the captured interaction partner. No higher MW bands were seen in conditions lacking Bhn.

FIG. 4(F) shows unprecedented Williamson C—O—C bond ether formation in an inter-molecular fashion between H3 proteins (Bhn4 in one linked to hydroxyl in another) which is driven by effective molarity, possibly suggesting a transient dimer model for KDM4A function.

FIG. 5 shows a number of functionalized protein residues which were successfully generated via the reaction methods described herein. Reagents and conditions used are provided in the examples section. FIG. 5(a) shows the residues generated via BACED reagents (embodiment (ii)/(iia)). FIG. 5(b) shows residues generated via the activated fluorinated radical precursors (embodiments (i), (ia), and (ib)) which can be distinguished as they contain at least one fluorine label on the γ carbon atom on the side chain.

FIG. 6 Shows reaction schemes according to various embodiments of the invention, as described in the examples.

FIG. 7(A) shows the method for expression of maltose binding protein in the presence of monoF-PySOOF-AA, as described in example 8.

FIG. 7(B) shows: Top—SDS-Page gel of the purification of MBP. Bottom—MS analysis of purified fractions demonstrating product and contaminant PylRS.

FIG. 8 shows reaction schemes according to various embodiments of the invention, as described in example 9.

FIG. 9 shows reaction schemes according to various embodiments of the invention, as described in example 10.

### DETAILED DESCRIPTION

The present invention provides a method for functionalizing a protein or peptide with a functional side chain moiety, wherein the protein or peptide comprises at least one singly occupied molecular orbital (SOMO) acceptor residue, which method comprises:
(c) contacting the protein or peptide with a specific radical precursor compound containing a functional group to be attached to the protein or peptide and a photocatalyst; and
(d) exposing the resultant composition to light radiation in order to provide a functionalized protein or peptide.

The SOMO acceptor residue is an amino acid residue situated in the peptide or protein, and linked to one or two adjacent residues by peptide bond(s). The SOMO acceptor residue comprises a group that is highly reactive towards C radical species, which group is a side chain having an alkene group. In some embodiments the SOMO acceptor residue may have a side chain of formula C₁₋₆ alkenyl. Preferably, the C═C double bond is at the terminal end of the alkenyl group. In a preferred embodiment the SOMO acceptor is dehydroalanine (Dha) or dehydrobutyrine (Dhb), preferably dehydroalanine.

The Dha residue may be introduced to the protein or peptide of interest by any suitable means, such as any of those set out in Chemical Science, Vol. 2, Number 9, Sept 2011, Pages 1617-1868 or in Current Opinion in Chemical Biology, Vol. 46, Oct 2018, Pages 71-81.

The residue to be functionalized may be at any suitable point in the protein or peptide chain.

### Embodiment (i) Aryl Sulfone Fluoride Derivatives (ASOOF)

In a first embodiment (i) of the above method the radical precursor compound is a compound of formula (II) below, referred to herein as an ASOOF precursor:

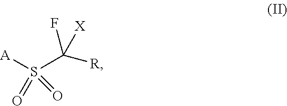

In the above formula (II), R is the functional side chain moiety which is attached to the protein or peptide via the group —CFX—.

A is an aryl or heteroaryl group, which is optionally substituted by one or more R₂ groups. Typically, A is unsubstituted or substituted with one, two or three R₂ groups, preferably A is unsubstituted or substituted with one or two R₂ groups. Most preferably A is unsubstituted.

R₂ is selected from the group consisting of halogen and C₍₁₋₆₎ alkyl which is unsubstituted or substituted with one or more groups (e.g. one, two or three, preferably one or two, groups) selected from hydroxy, oxy, halogen, amino, carboxy, C₍₁₋₆₎ ester, and C₍₁₋₆₎ ether. In some embodiments, R₂ is C₁₋₄ alkyl which is unsubstituted or substituted by hydroxy, oxy, halogen or amino. In a preferred embodiment A is unsubstituted.

In some embodiments A is a 6 membered ring.

In a preferred embodiment A is phenyl, pyridinyl, pyrimidinyl, benzothiazolyl, or pyrazinyl, more preferably A is pyridinyl, pyrimidinyl or benzothiazolyl.

In a most preferred embodiment, the compound of formula (II) is of formula (IIA) as set out below.

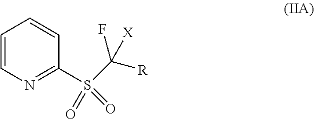

In the above formulae (II) and (IIA) X is selected from the list consisting of hydrogen, fluorine, chlorine, —COOH, and —CONH₂, preferably fluorine or hydrogen, most preferably fluorine.

In a most preferred embodiment the radical precursor compound is:

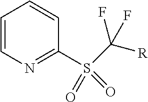

which is referred to herein as “pySOOF”.

In a further embodiment the radical precursor is

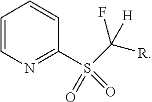

In a further embodiment the radical precursor compound is:

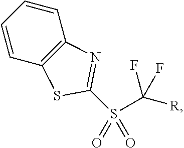

which is referred to herein as “BtSOOF”, or

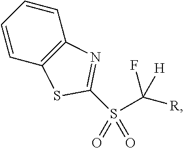

When compounds of formula (II) are used as the radical precursor compounds, the reaction composition must further comprise a source of Fe(II). The Fe(II) acts to reduce the photocatalyst to the active form which is capable of oxidising the radical precursor of formula (II), e.g. by reducing Ru(II) to Ru(I) as shown in FIG. 2(d). Additionally, the Fe(II) can act to reductively quench the radical protein/peptide intermediate generated by the initial reaction between the stabilised functional side chain radical and the SOMO acceptor residue. This has the benefit of preventing oxidative quenching of the intermediate which may otherwise arise due to an excess of oxidised photocatalyst, e.g. the Ru(II) catalyst species, and which leads to unwanted side products such as imine and hemiaminal formation (see FIG. 2(d)).

The source of Fe(II) is not particularly limited. In a preferred embodiment the source of Fe(II) is iron(II)sulfate, iron(II)trifluoromethylsulphonate (FeOTf₂), Fe(ClO₄)₂, FeF₂, or (NH₄)₂Fe(SO₄)₂, preferably iron(II) sulfate, e.g. FeSO₄·7H₂O.

The amount of Fe(II) compound used is not particularly limited, but may typically be from 1 to 1000 equivalents, preferably 5 to 600 equivalents, more preferably 10 to 300 equivalents, most preferably 25 to 250 equivalents relative to the amount of protein substrate used.

The amount of radical precursor compound used in this embodiment is not particularly limited, but may typically be from 0.1 to 1000 equivalents, preferably 0.5 to 250 equivalents, more preferably 0.5 to 50 equivalents, most preferably 2 to 25 equivalents relative to the amount of protein substrate used.

The reaction according to embodiment (i) may proceed according the scheme shown in FIG. 2(d). As can be seen, upon activation with light of the appropriate flux, the photo-excited oxidative state of the photocatalyst (e.g. Ru(II) photocatalyst) is reductively quenched by the Fe(II) to provide the active reduced species, e.g. (Ru(I)). The reduced species then reductively initiates the ASOOF precursor to yield a stabilized RCFX· radical species which then reacts via radical addition to the C═C double bond of the SOMO acceptor residue, such as Dha as shown below. The resulting α-carbon on-protein radical is then reduced via SET from Fe(II) to form an enolate intermediate that is protonated under the aqueous reaction conditions to yield the final functionalized protein/peptide.

### Embodiment (IA)

In a further specialized embodiment (ia) the same reaction conditions are used as for Embodiment (i) above, except that the group R in formula (II) is iodine, rather than a side chain group for attaching to the protein/peptide. Hence the radical precursor compound is a compound of formula

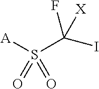

wherein A and X are as defined in embodiment (i) above. In a preferred embodiment A is pyridyl and X is fluorine, such that the above radical precursor is iodo-pySOOF.

Under the same reaction conditions as above for ASOOF, the reduced activated catalyst reductively activates the iodo-radical precursor to form a radical as shown below.

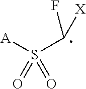

This stabilized radical species further reacts via radical addition to the C═C double bond of the SOMO acceptor residue via the same reaction pathway as set out above in the first aspect of this embodiment in order to generate a protein/peptide which is functionalised with a ASOOF radical precursor side chain.

This protein/peptide which has been functionalised with a ASOOF precursor side chain moiety may be activated via a photoredox catalyst and a source of Fe(II) using the same reaction conditions as embodiment (i) in order to provide a stabilized on-protein radical which may be used to conjugate it to further species. This moiety therefore allows for diverse further protein functionalization via various on-protein homolytic bond forming mechanisms, see FIG. 3(A).

### Embodiment (iai)

In a further embodiment the present invention provides a method of producing a protein/peptide comprising a residue containing an ASOOF functionalised side chain according to formula (IAi) below by incorporating a synthetic amino acid according to formula (IIi) into a protein/peptide. This may be done, for instance, using genetic code expansion techniques, such as those described in Example 8.

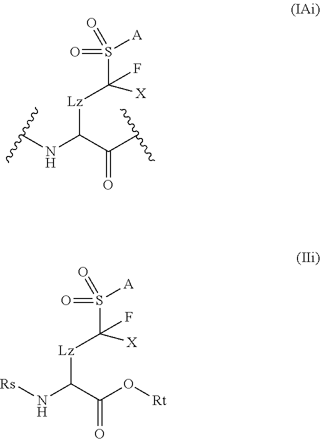

Wherein, in formulae (IAi) and (IIi), A and X are as defined in the above embodiments, and Lz is a C₁₋₄ alkyl linker group which may optionally be substituted with one or more groups selected from halogen, hydroxyl and amino. Lz is preferably methylene (—CH₂—), or —CH(CH₃)—. More preferably Lz is methylene. Rt is hydrogen or a protecting group, preferably hydrogen or C₁₋₄ alkyl, more preferably hydrogen or tert-butyl. Rs is hydrogen or a protecting group, more preferably hydrogen or tert-butoxycarbonyl (boc). In a preferred embodiment Rs and Rt are each hydrogen.

In preferred embodiments Lz is methylene, X is hydrogen or fluorine, A is heteroaryl selected from pyridinyl, pyrimidinyl or benzothiazolyl, and Rs and Rt are either both hydrogen, or are boc and tert-butyl, respectively. More preferably A is

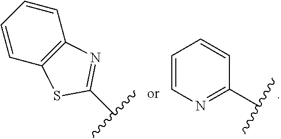 [SMILES: *c1nc2ccccc2s1 ; *c1ccccn1]

The protein/peptide which has been functionalized with an ASOOF precursor side chain moiety may be further activated/reacted as set out above for Embodiment (ia) above.

The present invention therefore also provides proteins/peptides according to formula (IAi) above, and synthetic amino acids according to formula (IIi) above. The present invention also provides salts of the compounds of formula (IIi) above.

### Embodiment (IB)

In a further specialized embodiment (ib) the same reaction conditions are used as for Embodiment (i) above, except that a radical precursor compound of formula (IV) is used.

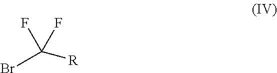

R is the functional side chain moiety which is attached to the protein or peptide via the group —CF₂—.

Under the same reaction conditions as above for ASOOF, the reduced activated catalyst reductively activates the precursor to form a radical as shown below.

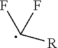

This stabilized radical species further reacts via radical addition to the C═C double bond of the SOMO acceptor residue via the same reaction pathway as set out above in the first aspect of this embodiment in order to generate a protein/peptide which is functionalised with the side chain —CF₂R.

### Embodiment (ii) Boronic Acid Catechol-Ester Derivatives (BACED)

In a further embodiment (ii) of the above method, the radical precursor compound is a compound of formula (III), herein referred to as a BACED reagent:

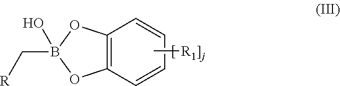

In formula (III), j is 0, 1, 2 or 3, typically j is 0, 1 or 2, preferably j is 0 or 1.

In a preferred aspect of embodiment (ii), the BACED reagent is of formula (IIIA) below.

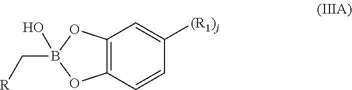

In formula (IIIA), j is 0 or 1.

Each R₁ in formulae (III) or (IIIA) above is independently selected from the group consisting of halogen and C₍₁₋₆₎ alkyl which is unsubstituted or substituted with one or more groups (e.g. one, two or three, preferably one or two, groups) selected from hydroxy, oxy, halo, amino, carboxy, C₍₁₋₆₎ ester, and C₍₁₋₆₎ ether. Preferably the group R₁ is C₍₁₋₄₎ alkyl, which is unsubstituted or substituted by one or two groups selected from hydroxy, halo, amino and carboxy. Most preferably, R₁ is hydrogen, CH₂CH₂NH₂, or CH₂CH(NH₂)COOH.

R is the functional side chain moiety which is attached to the protein or peptide via the group —CH₂—.

The BACED reagent should preferably have an oxidative half potential (Eₒₓ) of close to or less than that of the activated photocatalyst in order to be oxidised by said catalyst during the reaction.

The BACED reagent may be generated in situ by adding a functionalized boron compound and a catechol derivative represented by the formula (IIIB) below to the reaction mixture, wherein j and R₁ are as defined above.

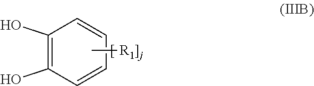

The functionalized boron compound may be any boron compound which is covalently bonded to the side chain to be attached to the protein or peptide (—CH₂R), i.e. any boron compound which comprises a B—CH₂—R unit. In order to form the active BACED reagent in situ, the boron compound should further be capable of substituting ligands in an aqueous environment. The boron component may be a boron salt, boronic acid and/or boronic ester. In one embodiment, the boron compound is a compound of formula [RCH₂BQ₃]V wherein each Q is independently a halogen, preferably chloro or fluoro, most preferably fluoro; and V is any suitable counterion such as K⁺, Li⁺, Na⁺, or NH₄⁺. In a further embodiment the boron compound is of formula RCH₂B(OR_{f})₂, wherein the R_{f} groups are independently hydrogen or C₁₋₆ alkyl or wherein the two R_{f} groups together form a straight or branched C₁₋₁₀ alkyl chain which links the two oxygen atoms in order to form a 4 to 7 membered ring together with the boron atom to which the oxygen atoms are attached. In a preferred embodiment the boron compound is RCH₂BF₃K, RCH₂B(OH)₂, or RCH₂Bpin, where pin is a pinacolato group bonded to the boron via the two oxygen atoms.

The amount of boron compound used is not particularly limited, but may typically be from 5 to 1000 equivalents, preferably 10 to 600 equivalents, more preferably 100 to 500 equivalents relative to the amount of protein substrate used. The amount of catechol derivative (IIIB) added is not particularly limited, but is preferably 0.02 equivalents or more, relative to the boron compound. In an embodiment the amount of catechol derivative is 0.02 equivalents or more, and 1 equivalent or less relative to the amount of boron compound added to the reaction mixture.

In embodiment (ii), the reaction may generally proceed according the scheme shown in FIG. 2(c). As can be seen, the photo-excited catalyst state, e.g. Ru(II)*, of the photocatalyst oxidatively initiates the BACED precursor to yield a RCH₂· radical species which then reacts via radical addition to the C═C double bond of the SOMO acceptor residue, such as Dha as shown in FIG. 2(c). The resulting α-carbon on-protein radical is then reductively quenched via SET from the reduced catalyst, e.g. Ru(I), to form an enolate intermediate that is protonated under the aqueous reaction conditions to yield the final functionalized protein/peptide.

### Embodiment (Iia) Boron Reagents

In one embodiment the present invention provides a method of functionalizing a protein or peptide with a functional side chain moiety, wherein the protein or peptide comprises at least one singly occupied molecular orbital (SOMO) acceptor residue as described herein.

The method comprises
(a) contacting the protein or peptide with a functionalized boron compound, a catechol derivative of formula (IIIB), and a photocatalyst having an oxidative half potential (Eₒₓ) of less than or equal to +1.2 V in its photo-activated state, when measured against a saturated calomel electrode as described in the embodiments above; and
(b) exposing the resultant composition to light radiation in order to provide a functionalized protein or peptide.

The functionalized boron compound and catechol derivative of formula (IIIB) are as defined in embodiment (ii) above.

Without wishing to be limiting, it is currently understood that the functionalized boron compound and catechol derivative of formula (IIIB) generate a BACED reagent of formula (III) in situ during step (a). However, the present invention is not restricted to methods in which the BACED reagent is formed (or is detectable) during the reaction and embodiments in which the BACED reagent either cannot be detected, or is not formed, are also encompassed within the scope of the invention.

### Reaction Conditions

Described below are reaction conditions for carrying out the methods of the invention. Unless stated otherwise, the aspects described below relate to all embodiments of the method of the invention, including methods wherein the radical precursor compound is of formula (II), (IIA), (III), (IIIA) or (IV), and methods wherein the reaction proceeds in the presence of a functionalized boron compound and a catechol derivative of formula (IIIB).

An advantageous feature of the present invention is that the reactions can be performed under mild redox conditions. Therefore, the photocatalysts used preferably have an oxidative half potential (Eₒₓ)* in their photo-actived oxidised state of less than or equal to +1.2 V, preferably less than or equal to +1.0 V, more preferably less than +1.0 V when measured against a saturated calomel electrode.

The photocatalysts used preferably also have a reductive half potential (E_{red}) in their reduced state of less than or equal to −1.5 V, preferably less than or equal to −1.4 V when measured against a saturated calomel electrode. For the avoidance of doubt, a lower reductive half potential as described herein is indicated by a lower negative, or higher positive value. Thus, a reductive half potential of −1.4 V is “less than” a reductive half potential of −1.5 V.

When the methods of embodiments (ii)/(iib) are used, the oxidative half potential (Eₒₓ) of the photocatalyst in its photoactivated state is preferably no more than 0.2 V less than the Eₒₓ of the radical precursor compound of formula (III) when measured against a saturated calomel electrode. Preferably the Eₒₓ of the photocatalyst is greater than the Eₒₓ of the radical precursor compound of formula (III) when measured against a saturated calomel electrode.

In some embodiments, the radical precursor compound of formula (III) may have an oxidative half potential (Eₒₓ) of +1.2 V or less, preferably +0.99 V or less, more preferably 0.8V or less, most preferably +0.5 V or less when measured against a saturated calomel electrode.

When the method of embodiments (i), (ia), or (ib) are used the photocatalyst preferably has an oxidative half potential Eₒₓ in its photoactivated oxidized state of greater than or equal to +0.72 V.

When the methods of embodiments (i), (ia), or (ib) are used, the reductive half potential (E_{red}) of the photocatalyst is preferably no more than 0.2 V less than the E_{red} of the radical precursor compound of formula (II)/(IV) when measured against a saturated calomel electrode. Preferably the E_{red} of the photocatalyst is greater than the E_{red} of the radical precursor compound of formula (II)/(IV) when measured against a saturated calomel electrode (i.e. is a stronger reductant).

In some embodiments, the radical precursor compound of formula (II)/(IV) may have a reductive half potential (E_{red}) of −1.4 V or less, preferably −1.2 V or less, more preferably −1.0 V or less when measured against a saturated calomel electrode.

The photocatalyst is preferably an Ru(II) or Ir(II) based catalyst, more preferably an Ru(II) catalyst. In a particularly preferred embodiment the photocatalyst is Ru(bpy)₃Cl₂ or Ru(bpm)₃Cl₂.

The amount of photocatalyst used is not particularly limited, but may be substochiometric with respect to the amount of protein/peptide. In some embodiments, the amount of photocatalyst is 0.1 to 100 equivalents, preferably 0.1 to 10, more preferably 0.25 to 1 equivalents with respect to the amount of protein or peptide.

Light radiation of appropriate flux is used in order to activate the photocatalyst, e.g. a Ru(II) photocatalyst, and therefore initiate radical formation and the coupling reaction. This has the advantages of allowing temporal, spatial and kinetic control of the reaction. Further the light can be tissue penetrating and can be benign, so as to not damage the sample, e.g. protein/peptide or tissue. The light radiation is preferably visible light. In some embodiments the light radiation has a wavelength in the region of 300 nm to 600 nm, preferably 400 to 500 nm. In a further embodiment, the light radiation has a wavelength in the range of 430 to 470 nm.

The light intensity is not particularly limited, but in some embodiments the light provided to the reaction may be 0.1 to 1000 W, preferably 1 to 200 W, more preferably 1 to 100 W, yet more preferably 5 to 60 W. In a preferred embodiment the light intensity provided to the reaction is 45 to 55 W.

The use of light activation in order to initiate the protein functionalization reactions described herein allows for precise spatiotemporal control of the reactions. The timed and targeted use of such a potentially tissue-penetrating trigger could be used to modify and probe complex biological systems.

The present reactions may be performed without the need for harsh solvents which may damage proteins. The solvent used is preferably water.

The reactions are preferably performed under anaerobic conditions in order to avoid unwanted oxidation reactions with the radical intermediates.

The reaction may advantageously be performed under mild pH conditions. Preferably the reaction is performed at a pH of 5.0 to 9.0. More preferably the reaction is performed at a pH of 5.5 to 8.5. In one embodiment the reaction is performed at a pH of 5.0 to 7.0. In a further embodiment the reaction is performed at pH 5.5 to pH 6.5.

The reaction mixture may optionally further comprise one or more additional components, such as buffer to modulate the pH. In some embodiments the buffer is selected from sodium phosphate buffer (NaPi), HEPES, FPBS, phosphate buffer saline (PBS), NH₄OAc, guanadinium chloride and combinations thereof. Preferably the buffer is a combination of NH₄OAc, and guanadinium chloride.

The reaction is typically carried out at a temperature of from 0 to 50° C., preferably from 5 to 40° C., more preferably from 10 to 30° C., and most preferably from 15 to 25° C.

The present invention further allows for rapid reaction times. The duration of the reaction is typically less than 4 hours, preferably less than 1 hour, more preferably less than 30 minutes, yet more preferably less than 20 minutes, and most preferably less than 15 minutes.

### Functional Side Chain Moieties

Described below are functional side chain moieties which can be attached to proteins or peptides using the methods of the invention. Unless stated otherwise, the side chains described below can be added using any embodiment of the method of the invention, including methods wherein the radical precursor compound is of formula (II), (IIA), (III), (IIIA) or (IV), and methods wherein the reaction proceeds in the presence of a functionalized boron compound and a catechol derivative of formula (IIIB).

Those skilled in the art will appreciate that the functional side chain moiety attached to the protein or peptide may provide a variety of functions, such as aiding in enzymatic or other biological studies, linking it to specific payloads, and modulating its chemical properties.

The present methods allow functional side chain moieties to be attached to proteins or peptides via a light mediated radical reaction under mild conditions. In general, the group R will be attached to the protein/peptide via the group —CXF— where the ASOOF precursor is used (embodiment (i), first aspect), via the group —CF₂— where the precursor of formula (IV) is used and via the group —CH₂— when the boron-containing precursors are used (embodiment (ii), (iia)). Those skilled in the art will appreciate that there is no particular limitation to the group R which may be attached to the protein or peptide, as the methods described herein are generally applicable and can be used even where reactive groups are present. The group attached to the protein or peptide may therefore comprise any suitable chemical moiety that is useful for attachment. This group may, for example, comprise a linker group comprising a payload and/or a reactive functional group that is capable of attaching to a payload via a further reaction. Such linkers, payloads, and reactive functional groups, are well known in the field of protein conjugates.

In a first aspect of the above embodiments the group R represents a payload which is optionally connected via a linker. The payload may be selected from the group consisting of pharmaceutical drugs, sugars, polysaccharides, peptides, proteins, vaccines, antibodies, nucleic acids (DNA, RNA), viruses, labelling compounds, stabilized radical precursors, biomolecules and polymers, any of which may optionally be connected via a linker group.

In one embodiment the payload is selected from pharmaceutical drugs, sugars, polysaccharides, peptides, proteins, antibodies, labelling compounds, stabilized radical precursors, and polymers. In a further embodiment the payload is selected from peptides, proteins, sugars, polysaccharides, labelling compounds and polymers. Preferably, the payload is a sugar or a labelling compound. In a particular embodiment the payload may be an amino acid, which may be either a natural or synthetic amino acid, and which may optionally be attached via a covalent bond to its side chain.

The linker group L can be substantially any suitable multivalent organic group, typically being divalent or trivalent. In one embodiment, the linker group L may be an organic group having a molecular weight of 2000 or less, preferably 1500 or less, and more preferably 1000 or less. The linker may optionally comprise polyethylene glycol (PEG) or a PEG analogue. Suitable PEG analogues include those listed in Chemical Society Reviews, Vol. 47, Number 24, 21 Dec. 2018, Pages 8971-9160. For the avoidance of doubt, when the linker is described as “alkyl” or other related terms, it is to be interpreted as covering a multivalent group, e.g. a divalent “alkenyl” group.

In a preferred embodiment the linker is a group L1, which is selected from: alkyl in which one or more non-adjacent carbon atoms may be optionally substituted for (i.e. replaced with) a group selected from NH, O, S, —C(O)NH— or —NHC(O)—; polyethyleneglycol (PEG) and analogues thereof, saccharides; polysaccharides; polyglycine; polyamide; and combinations of two or more of these groups.

In a preferred embodiment L1 is selected from alkyl in which one or more non-adjacent carbon atoms may be optionally substituted for a group selected from NH, O, S, —C(O)NH— or —NHC(O)—; PEG, PEG analogues, polyamides, and combinations of two or more of these groups. The alkyl group is typically C₁₋₂₀ alkyl, preferably C₁₋₁₀ alkyl, more preferably C₁₋₆ alkyl.

In a preferred embodiment L1 is PEG or C₁₋₂₀ alkyl in which one two or three non-adjacent carbon atoms may be optionally substituted for a group selected from NH, O, S, —C(O)NH— or —NHC(O)—. Preferably, L1 is C₁₋₁₀ alkyl, more preferably C₁₋₆ alkyl.

Suitable polymers for attaching to the present invention include natural polymers such as polypeptides, polysaccharides, polynucleotides and polymeric lipids as well as synthetic polymers. Preferred polymers include PEG, PEG analogues, polyamides, polyacrylamides, and polyacrylates, as well as RAFT (reversible addition-fragmentation chain transfer polymerization) generated polymers. Further preferred examples of polymers which may be attached as the payload include those set out in Chemical Society Reviews, Vol. 47, Number 24, 21 Dec. 2018, Pages 8971-9160. The polymers typically have a molecular mass of less than 10 kDa, preferably less than 5 kDa, more preferably less than 2 kDa, most preferably less than 1 kDa. In a preferred embodiment the polymer is PEG, a PEG analogue, a polyacrylamide or a polyacrylate, more preferably the polymer is PEG.

The payload attached to the protein or peptide may optionally be a labelling compound, which is herein defined as a compound comprising a labelling group allowing for its detection in chemical and/or biological studies. Suitable labels include isotopic labels wherein one or more atoms in the group are labelled with a particular isotope which may be detected via suitable means such as NMR, mass spectrometry and radiolabeling studies. Suitable labelling isotopes include deuterium, ¹⁹F, ¹³C, and ¹⁵N. Suitable labelled groups include biomolecules, sugars, and natural or synthetic amino acids, which have been labelled with one or more of the above isotopes in a particular location. Additionally, the term labelling group is intended to cover other payloads or side chain moieties as described herein which have been labelled with a particular isotopic label, as defined above. Other suitable labelling compounds include fluorophores and FRET reagents. Further, suitable labelling compounds include compounds which may assist in the identification and or isolation of the peptide of interest. In one embodiment the labelling compound is a FLAG-tag or biotin. In a preferred embodiment the labelling compound is biotin, which may be attached via its terminal carboxy group, e.g. in the form of an ester.

In one aspect of methods (i), (ia), (iai) and (ib) of the present invention and the products thereof, the functional side chain moiety R is attached to the protein or peptide via an ¹⁹F containing linking group —CFX— or —CF₂—. This group allows the monitoring of various reaction pathways through NMR, as demonstrated in example 7.

In a further aspect of methods (i), (ia), (iai) and (ib) of the present invention and the products thereof, the functional side chain moiety R is attached to the protein or peptide via an ¹⁸F containing linking group —CFX— or —CF₂—, i.e. one or both of the fluorine atoms bonded to the linking carbon atom may be ¹⁸F. This group allows labelling of peptides/proteins, which may allow for the monitoring of various reaction pathways as demonstrated in, e.g., example 9.

In some embodiments, either one or both, preferably one, of the fluorine atoms bonded to the carbon adjacent to the group R is ¹⁸F in any of the compounds of formulae (II), (IV), (IA), (IIi) and the iodo compound used as a radical precursor in embodiment (ia).

A biomolecule or biological molecule is defined herein as a molecule present in organisms that is essential to one or more biological processes. This term is intended to cover small organic molecules, typically with molecular masses of less than 5 kDa, preferably less than 1.5 kDa, such as primary metabolites, secondary metabolites and natural products which are used in essential biological processes. This term includes endogenous and exogenous biomolecules, such as metabolites, vitamins and other organic nutrients.

As used herein, a stabilized radical precursor refers to a functional group which may be used to generate a radical for further reactions, e.g. by stimulation with light radiation.

Suitable groups include groups of formulae

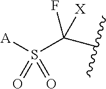

wherein A and X are as defined above in relation to embodiment (i).

As used herein, the term “pharmaceutical drug” refers to a chemical compound which has known biological effect on an animal, such as a human. Typically, drugs are chemical compounds which are used to treat, prevent or diagnose a disease. Preferred drugs are biologically active in that they produce a local or systemic effect in animals, preferably mammals, more preferably humans. Typically, the drug molecule has M_{w} less than or equal to about 5 kDa. Preferably, the drug molecule has M_{w} less than or equal to about 1.5 kDa.

A more complete, although not exhaustive, listing of classes and specific drugs suitable for use in the present invention may be found, for example, in each of: (a) Pharmaceutical Substances: Syntheses, Patents, Applications, Axel Kleemann and Jurgen Engel (Thieme Medical Publishing, 1999) and (b) The Merck Index: An Encyclopedia of Chemicals, Drugs, and Biologicals, ed. S Budavari et al. (CRC Press, 1996); the contents of which are incorporated herein by reference in their entirety.

As used herein, sugar covers monosaccharides, including glucose, fructose, galactose, ribose and deoxyribose as well as disaccharides, which are composed of two monosaccharides joined by a glycosidic bond, including sucrose, lactose, and maltose. As used herein the term polysaccharide is intended to cover polymers of more than two saccharide molecules joined by glycosidic bonds, and includes, e.g. starch, cellulose and chitin. Any saccharide which forms part of a sugar or polysaccharide as used herein may be a modified saccharide, for example wherein the hydroxyl group of the natural sugar is replaced with a substituent. Acetyl, N-acetyl and methyl groups are examples of common substituents. Alternatively, hydroxyl groups may be absent, e.g. replaced by a hydrogen atom. Thus, saccharides, sugars and polysaccharides described herein may be unsubstituted, or substituted by one or more, typically 1 or 2, acetyl groups or N-acetyl groups. Thus, the term sugar as used herein covers groups such as N-acetylglucosamine.

As used herein, the term “peptides” refers to biologically occurring or synthetic short chains of at amino acid monomers linked by peptide (amide) bonds. The covalent chemical bonds are formed when the carboxyl group of one amino acid reacts with the amino group of another. The shortest peptides are dipeptides, consisting of 2 amino acids joined by a single peptide bond, followed by tripeptides, tetrapeptides, etc. A polypeptide is a continuous peptide chain comprising multiple amino acids.

As used herein, the term “proteins” refers to biological molecules comprising polymers of amino acid monomers which are distinguished from peptides on the basis of size, and as an arbitrary benchmark can be understood to contain approximately 50 or more amino acids.

Proteins consist of one or more polypeptides arranged in a biologically functional way, often bound to ligands such as coenzymes and cofactors, or to another protein or other macromolecules (DNA, RNA, etc.), or to complex macromolecular assemblies.

In a further aspect of the embodiments of the invention, R is a functional group R^{F}; or one or more, typically 1 or 2, functional groups R^{F} connected via a linker group of formula L2. R^{F} is
hydrogen, C₃₋₁₀ cycloalkyl, aryl or heteroaryl; wherein the cycloalkyl, aryl and heteroaryl groups are unsubstituted or substituted by one or more groups selected from ═O, ═NR^{a}, Y and (C₁₋₆ alkyl)-Y; or
a reactive group Y selected from C₂₋₆ alkenyl, C₂₋₆ alkynyl, halogen, hydroxy, —OR, —SR^{a}, —S(O)R^{a}, —S(O)₂R^{a}, —OSO₃R^{a}, —NR^{a}C(O)R^{b}, —NR^{a}CO₂R^{b}, —NHC(O)NR^{a}R^{b}, —NHCNH₂NR^{a}R^{b}, —NR^{a}SO₂R^{b}, —N(SO₂R^{a})₂, —NHSO₂NR^{a}R^{b}, —OC(O)R^{a}, —C(O)R^{a}, —CO₂R^{a}, —C(O)NR^{a}R^{b}, —C(O)(NHNH₂), —ONH₂, —C(O)N(OR^{a})R^{b}, —SO₂NR^{a}R^{b} or —SO(NR^{a})R^{b}; cyano, nitro, C₁₋₆ azidoalkyl, —NR^{a}R^{b} and —(NR^{a}R^{b}R^{c})⁺;

wherein:

R^{a}, R^{b}, and R^{c} independently in each instance represent hydrogen, C₁₋₆ alkyl, C₃₋₁₀ cycloalkyl, heterocyclyl, phenyl, benzyl and heteroaryl, wherein the alkyl, cycloalkyl, heterocyclyl, phenyl, benzyl and heteroaryl groups at R^{a}, R^{b}, and R^{c} are unsubstituted or substituted by one or more substituents selected from halogen, hydroxy, ═O, —NH₂, —SO₃— and C₁₋₆ alkoxy; and

L2 is selected from alkyl in which one or more non-adjacent carbon atoms may be optionally substituted for (i.e. replaced with) a group selected from NH, O, S, —C(O)NH— or —NHC(O)—; polyethyleneglycol (PEG) and analogues thereof, saccharides; polysaccharides; polyglycine; polyamides; or combinations of two or more of these groups.

In a preferred embodiment, L2 is selected from alkyl in which one or more non-adjacent carbon atoms may be optionally substituted for a group selected from NH, O, S, —C(O)NH— or —NHC(O)—; PEG; PEG analogues; saccharides; polyamides and combinations of two or more of these groups. The alkyl group is typically C₁₋₂₀ alkyl, preferably C₁₋₁₀ alkyl, more preferably C₁₋₆ alkyl. The saccharide is typically glucose, galactose, ribose or deoxyribose.

In a preferred embodiment L2 is PEG, a saccharide, C₁₋₂₀ alkyl in which one two or three non-adjacent carbon atoms may be optionally substituted by a group selected from NH, O, S, —C(O)NH— or —NHC(O)—, or combinations of two or more of these groups.

In a preferred embodiment L2 is PEG or C₁₋₂₀ alkyl in which one two or three non-adjacent carbon atoms may be optionally substituted by a group selected from NH, O, S, —C(O)NH— or —NHC(O)—.

In a further embodiment, L2 is C₁₋₁₀ alkyl, preferably C₁₋₆ alkyl.

In a particularly preferred embodiment L2 is C₁₋₄ alkyl, such as methylene, ethylene or propylene, preferably methylene or ethylene.

Typically, R is the functional group R^{F}, or a group -L2-R^{F}.

In a further embodiment R is -L2(R^{F})₂.

In some embodiments, R is an amino acid, which is covalently attached via its side chain.

In particular, R may have a structure as set out below, wherein Lz, Rs and Rt are as defined in embodiment (iai) above.

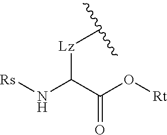

Typically, R^{F} is
hydrogen, C₃₋₆ cycloalkyl, phenyl or pyridyl; wherein the cycloalkyl, phenyl and pyridyl groups are unsubstituted or substituted by one or two groups selected from ═O, ═NR^{a}, Y and —(C₁₋₆ alkyl)-Y; or
a reactive group Y selected from C₂₋₆ alkenyl, C₂₋₆ alkynyl, halogen, hydroxy, —OR^{a}, —SR^{a}, —S(O)R^{a}, —S(O)₂R^{a}, —NR^{a}C(O)R^{b}, —OC(O)R^{a}, —C(O)R^{a}, —CO₂R^{a}, —C(O)NR^{a}R^{b}, —C(O)(NHNH₂), —ONH₂, C₁₋₆ azidoalkyl, —NR^{a}R^{b} and —(NR^{a}R^{b}R^{c})⁺.

Preferably R^{F} is
hydrogen, cyclohexyl, phenyl; or
a reactive group Y selected from C₂₋₆ alkenyl, C₂₋₆ alkynyl, halogen, —S(O)₂R^{a}, —NR^{a}C(O)R^{b}, —OC(O)R^{a}, —C(O)R^{a}, —CO₂R^{a}, —C(O)NR^{a}R^{b}, C₁₋₆ azidoalkyl, —NR^{a}R^{b} and —(NR^{a}R^{b}R^{c})⁺.

In one embodiment, R^{F} is a reactive group Y selected from C₂₋₆ alkenyl, C₂₋₆ alkynyl, halogen, —S(O)₂R^{a}, —NR^{a}C(O)R^{b}, —OC(O)R^{a}, —C(O)R^{a}, —CO₂R^{a}, —C(O)NR^{a}R^{b}, C₁₋₆ azidoalkyl, —NR^{a}R^{b} and —(NR^{a}R^{b}R^{c})⁺. In one aspect of this embodiment, R^{F} is a reactive group Y selected from C₂₋₆ alkenyl, C₂₋₆ alkynyl, halogen and C₁₋₆ azidoalkyl.

In particular, R is a group Y or L2-Y, wherein L2 is C₁₋₄ alkyl, such as methylene, ethylene or propylene, preferably methylene or ethylene, and Y is selected from C₂₋₆ alkenyl, C₂₋₆ alkynyl, halogen, —S(O)₂R^{a}, —NR^{a}C(O)R^{b}, —OC(O)R^{a}, —C(O)R^{a}, —CO₂R^{a}, —C(O)NR^{a}R^{b}, C₁₋₆ azidoalkyl, —NR^{a}R^{b} and —(NR^{a}R^{b}R^{c})⁺, preferably Y is selected from C₂₋₆ alkenyl, C₂₋₆ alkynyl, halogen and C₁₋₆ azidoalkyl.

Typically, R^{a}, R^{b}, and R^{c} independently in each instance represent hydrogen, C₁₋₆ alkyl, 5- to 6-membered heterocyclo, phenyl, benzyl and 5- to 6-membered heteroaryl, for example hydrogen, C₁₋₆ alkyl, phenyl, benzyl or pyridyl; wherein the alkyl, heterocyclo, phenyl, benzyl and heteroaryl groups at R^{a}, R^{b}, and R^{c} are unsubstituted or substituted by one or more substituents selected from halogen, hydroxy, ═O, —NH₂, —SO₃— and C₁₋₆ alkoxy. The groups R^{a}, R^{b}, and R^{C} when present may be the same or different. In one preferred embodiment, when multiple R^{a}, R^{b}, and R^{c} groups are attached to the same Y moiety, one of the groups is as defined according to any of the above definitions, whilst the other R^{a}, R^{b}, and R^{c} groups attached to the moiety are selected from hydrogen and C₁₋₃ alkyl.

In some embodiments R^{a} may be hydrogen or C₁₋₄ alkyl.

In some embodiments R^{b} may be hydrogen or C₁₋₄ alkyl.

In some embodiments R^{C} may be hydrogen or C₁₋₃ alkyl.

In a particular embodiment, R is the functional group R^{F}; or one or more, preferably one, functional groups R^{F} connected via the linker group L2, wherein R^{F} is a reactive moiety Y selected from: C₂₋₆ alkenyl, C₂₋₆ alkynyl, halogen, —OC(O)R^{a}, —C(O)R^{a}, —CO₂R^{a}, —C(O)(NHNH₂), —ONH₂ and C₁₋₆ azidoalkyl; or R contains a reactive moiety of formula

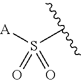

wherein A is as defined above; and wherein the reactive moiety

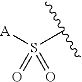

may optionally be connected via a linker group L2. In a preferred aspect of the above embodiment L2 is an alkyl group in which one or more non-adjacent carbon atoms may be optionally substituted for a group selected from NH, O, S, —C(O)NH— or —NHC(O)—. In a more preferred aspect of the above embodiment, L2 is C₁₋₄ alkyl, such as methylene or ethylene.

The reactive moiety in the above embodiment is optionally selected from halogen, C₁₋₆ azido, C₂₋₆ alkynyl,

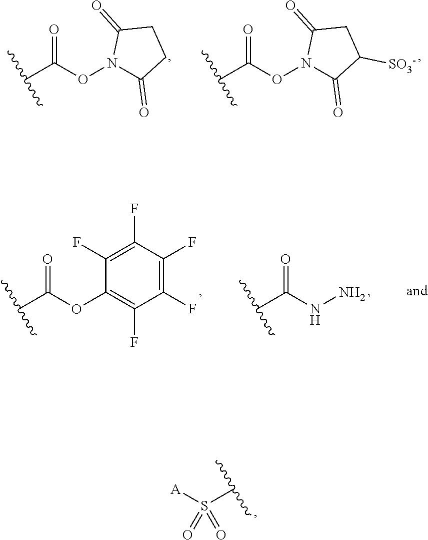

preferably

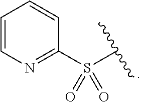 [SMILES: *S(=O)(=O)c1ccccn1]

The reactive moiety in the above embodiment is preferably selected from halogen, C₁₋₆ azido, C₂₋₆ alkynyl and

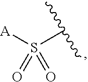

preferably

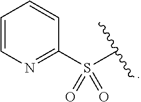 [SMILES: *S(=O)(=O)c1ccccn1]

In a further preferred aspect of any of the above embodiments the group L2-R^{F} is C₁₋₃ haloalkyl, preferably C₁₋₃ iodoalkyl or C₁₋₃ bromoalkyl.

In one embodiment, when the group R is -L2(R^{F})₂, L2 is C₁₋₄ alkyl, the first R^{F} is —CO₂R^{a}, and the second R^{F} is —NR^{a}R^{b} or —NH-Boc, wherein Boc is the protecting group tert-butoxycarbonyl. Preferably in said embodiment L2 is C₂ alkyl, the first R^{F} group is —CO₂H, and the second R^{F} is —NH₂.

In one embodiment R is the group -L2-Y, wherein Y is hydroxyl, —OR^{a}, —NR^{a}C(O)R^{b}, —NR^{a}R^{b} and —(NR^{a}R^{b}R^{c})⁺; wherein L2 is C₁₋₃ alkyl, preferably methylene or ethylene.

In another embodiment R is —SR^{a}, —S(O)R^{a}, —S(O)₂R^{a}, —C(O)R^{a}, —CO₂R^{a}, —C(O)NR^{a}R^{b}.

The groups R^{a}, R^{b} and R^{c} are as defined above.

In a preferred embodiment the side chain R corresponds to that used in any of Examples 1a to 2ag.

In a further embodiment the side chain is any group R which, together with the —CH₂—, —CXF— or —CF₂— linking group (from formula (III), (II) or (IV), respectively) and the residue to which it is bonded form one of the natural amino acids, except that the the γ carbon of the residue is substituted by one or two fluorines as applicable.

The functional group R^{F} may be attached at any appropriate point to the linker group, preferably at the terminal position, such as the terminal carbon.

### Embodiment (i) Functional Side Moieties

When the method of embodiment (i) is used it has been found that use of certain halo compounds as the group R can lead to side reactions where the groups other than R are added to the protein or peptide. Therefore, in a preferred aspect of this embodiment where R is halogen, it is fluorine.

In cases where the group X is hydrogen, R is preferably a group capable of stabilizing the intermediate wherein the radical is situated on the adjacent carbon. In a preferred aspect of this embodiment R is halogen, hydroxy, —OR^{a}, —SR^{a}, —SOR^{a}, —SO₂R^{a}, —OSO₃R^{a}, —NR^{a}COR^{b}, —NR^{a}CO₂R^{b}, —NHCONR^{a}R^{b}, —NHCNH₂NR^{a}R^{b}, —NR^{a}SO₂R^{b}, —N(SO₂R^{a})₂, —NHSO₂NR^{a}R^{b}, —OCOR^{a}, —COR^{a}, —CO₂R^{a}, —CONR^{a}R^{b}, —CON(OR^{a})R^{b}, —SO₂NR^{a}R^{b} or —SO(NR^{a})R^{b}.

Preferably R is —CO₂R^{a}, or —CONRₐR_{b} most preferably R is —COOH.

The groups R^{a}, R^{b} and R^{c} are as defined above.

### Embodiment (Ib) Functional Side Moieties

When the radical precursor compound of formula (IV) is used (embodiment (ib)) R is selected from —COOR^{d} and —CONR^{d}R^{e} wherein R^{d} represents hydrogen, C₁₋₆ alkyl, C₃₋₁₀ cycloalkyl, heterocyclo, phenyl, benzyl and heteroaryl, wherein the alkyl, cycloalkyl, heterocyclyl, aryl and heteroaryl groups at R^{d} are unsubstituted or substituted by one or more substituents selected from halogen, hydroxy, ═O, —NH₂, C₁₋₆ alkoxy and —NHCOR^{e}; and R^{e} represents hydrogen or C₁₋₄ alkyl, preferably hydrogen.

Preferably R^{d} represents hydrogen, C₁₋₆ alkyl, or a 5 or 6 membered heterocyclyl, wherein said alkyl or heterocyclyl, groups are unsubstituted or substituted by one or more substituents selected from hydroxy, —NH₂, C₁₋₆ alkoxy and —NHCOR^{e}.

In one embodiment R^{d} is hydrogen or C₁₋₆ alkyl which is unsubstituted, or substituted by 1 or 2 substituents selected from hydroxy, —NH₂, and C₁₋₆ alkoxy.

In a further preferred embodiment R is —C(O)OH, —CONH₂ or -GlcNAc.

### Embodiments (ii) and (Iia) Functional Side Moieties

When the reaction method of embodiments (ii) or (iia) are used, it is preferred that R comprises a moiety which stabilizes the radial intermediate, such as an adjacent electron withdrawing group.

In one aspect of embodiments (ii) and (iia), R is a functional group R^{F}; or one or more, typically 1 or 2, functional groups R^{F} connected via a linker group of formula L2.

R^{F} is
C₃₋₁₀ cycloalkyl, heteroaryl; wherein the cycloalkyl and heteroaryl groups are unsubstituted or substituted by one or more groups selected from ═O, ═NR^{a}, Y and (C₁₋₆ alkyl)-Y; or
a reactive group Y selected from, C₂₋₆ alkynyl, halogen, —SR^{a}, —S(O)R^{a}, —S(O)₂R^{a}, —OSO₃R^{a}, —NR^{a}C(O)R^{b}, —NR^{a}CO₂R^{b}, —NHC(O)NR^{a}R^{b}, —NHCNH₂NR^{a}R^{b}, —NR^{a}SO₂R^{b}, —N(SO₂R^{a})₂, —NHSO₂NR^{a}R^{b}, —OC(O)R^{a}, —CO₂R^{a}, —C(O)NR^{a}R^{b}, —C(O)(NHNH₂), —ONH₂, —C(O)N(OR^{a})R^{b}, —SO₂NR^{a}R^{b} or —SO(NR^{a})R^{b}; cyano, nitro, C₁₋₆ azidoalkyl, —NR^{a}R^{b} and —(NR^{a}R^{b}R^{c})⁺.

In this aspect the reactive group Y may be selected from halogen, C₁₋₆ azido, C₂₋₆ alkynyl,

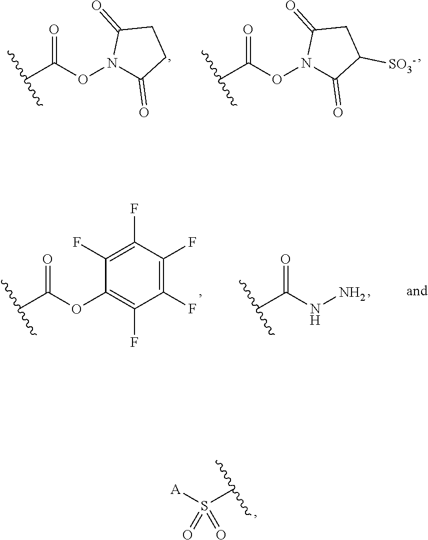

preferably

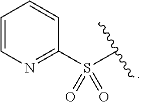 [SMILES: *S(=O)(=O)c1ccccn1]

In a further aspect of this embodiment R is C₁₋₆ halo, preferably C₁₋₆ bromo or C₁₋₆ iodo.

Where L2, R^{a}, R^{b}, and R^{c} are as defined in any of the embodiments above.

In preferred aspects of embodiments (ii) and (iia) R is not methyl, tert-butyl, propeneyl, phenyl or —C(O)R_{g} where R_{g} is

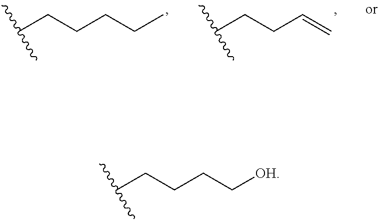

In a further embodiment, R is not —C(O)Rₕ, where Rₕ is C₁₋₆ alkyl or C₂₋₆ alkenyl, optionally substituted by one or more hydroxy groups.

In the embodiments (i), (ia) and (ib), the fluorine groups present in the radical precursor compound act as stabilising groups.

### Further Reactions of Side Chains

In some embodiments the functional side chain which is attached to the protein or peptide is a group capable of undergoing further reactions, in order to modify it, or to attach it to one or more further molecules. Therefore, in an embodiment, the present invention also provides a method as defined above where the functional side chain added to the protein is further reacted to modify it, or attach it to a further molecule.

The R groups attached to the protein or peptide as described above may be further reacted via any suitable reactions, for instance to attach them to one or more further molecule of interest. The further reactions are preferably biocompatible reactions, i.e. reactions which can be performed with minimal damage to the protein or peptide, e.g. under aqueous conditions without needing excessive temperatures. In a preferred embodiment the group R comprises one or more of the reactive moieties described above, which may be reacted via further linking reactions as described below. The person skilled in the art would be aware of suitable linking reactions such as, e.g. standard “click chemistry” reactions via azido, alkynyl and reactive esters, e.g. NHS esters.

The further molecule which may be attached to the reactive functional side chain moiety of the functionalized protein/peptide is not particularly limited, but includes pharmaceutical drugs, sugars, polysaccharides, peptides, proteins, vaccines, antibodies, nucleic acids, viruses, labelling compounds, biomolecules and/or polymers. In a preferred embodiment the further molecule is a drug, sugar, peptide, protein antibody biomolecule or polymer, preferably a peptide, protein or polymer. These terms are as defined above in relation to the functional side chain moiety, or in the definitions below.

In one embodiment, when the R group contains a suitable electrophile, such as a halogen it may react with a nucleophile, e.g. an off-protein nucleophile, via nucleophilic substitution by displacing a suitable leaving group such as a halogen. For instance, where the R group contains a halogen moiety, preferably a terminal halogen, it may be reacted via suitable chemistries to create C—S, bonds by reacting with nucleophiles such as a thiol e.g. beta-mercaptoethanol; to create C—P bonds, e.g. by reacting with TCEP (tris(2-carboxyethyl)phosphine); or C—N bonds, e.g. by reacting with methylamines, or N₃⁻. Alternatively, the electrophile containing R group may be reacted with a suitable nucleophile on a further protein or peptide such as a cysteine or lysine side chain in order to attach the protein or peptide to a further protein or peptide. By tuning the pH, off protein nucleophile concentration, and halogen choice it is possible to selectively facilitate intermolecular nucleophile substitution at C-Halogen bonds wile avoiding competing side reactions such as elimination and intraprotein nucleophilic substitution.

In a further embodiment, a halogen present on the R group may be substituted for alternative halogen groups, e.g. I to Cl, or Br to Cl, via a Finkelstein type reaction. This may be done in addition to, or prior to attaching the R group to a further molecule of interest e.g. via nucleophilic substitution.

In a further embodiment, where the side chain itself contains a stabilized radical precursor, such as the

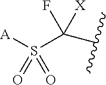

group described above, it may be activated to provide an “on-protein” radical on the protein or peptide in question, as described in example 4 (FIG. 3), using reaction conditions as described above, e.g. light radiation, a photocatalyst and source of Fe(II). The on-protein radical may be further reacted with any suitable group containing a SOMO acceptor moiety, such as an alkene group. For example, the on-protein radical may be reacted with a SOMO acceptor residue, e.g. a protein or peptide having a side chain containing an alkene group, e.g. a C₁₋₆ alkene groups, for example a further protein or peptide containing a Dha or Dhb residue, in order to provide a site-selective bond between the functionalized protein/peptide and a further protein/peptide.

Alternatively, the on-protein radical may be reacted with a suitable alkene containing monomer units in order to provide radical initiated polymerization on the protein/peptide. For instance, the functionalized protein or peptide may be reacted with a monomer of general formula

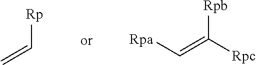

to provide a further functionalized protein or peptide containing at least one functionalized residue of formula (IP) below.

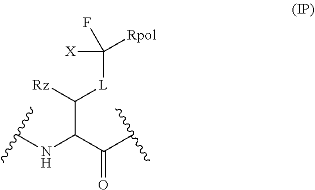

Wherein, L is a linker group as defined above or a bond; R_{z} is hydrogen or methyl, preferably hydrogen; and X is as defined above.

In the case where the monomer used is

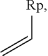

the group Rpol is

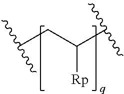

where q is typically 1 to 20, preferably 1 to 10, more preferably 1 to 5, most preferably 1, 2 or 3.

In the case where the monomer

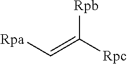

is used, the group Rpol is instead

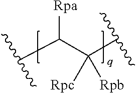

The pendant groups Rpb and Rpc may in some embodiments be joined together to form a ring. The polymer groups Rpol described above may be terminated by any suitable group such as hydrogen.

In an embodiment the generated on-protein radical may be reacted with one or more monomers of formula,

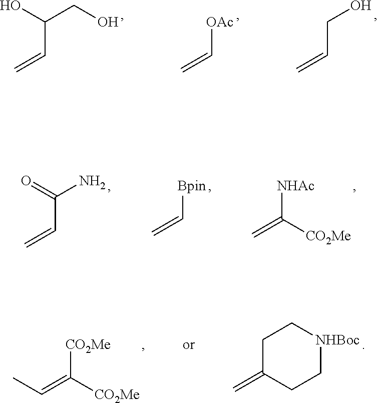

In an alternative embodiment, the on-protein radical generated may be reacted with a further radical terminating group, as shown in FIG. 3, such as hydroxy-TEMPO, or a diselenium compound of formula Rₕ—Se—Se—Rₕ, where each Rₕ is C₁₋₆ alkyl, C₁₋₆ cycloalky, or C₁₋₆ aryl, preferably phenyl. In such embodiments, the further functionalized protein or peptide produced may contain at least one functionalized residue according to formula (IP) above, except that the group Rpol is replaced by Rrad, wherein Rrad is a radical terminating group, which may be —Se—Rₕ, or

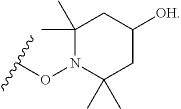 [SMILES: *ON1C(C)(C)CC(O)CC1(C)C]

method according to any one of claims 3 to 6, wherein when the functional side chain moiety comprises a reactive moiety as defined in one of claim 4 to 6, the method further comprises reacting the peptide or protein via one of the reactive moieties to connect the functional side chain to a further molecule.

### Functionalized Proteins and Peptides

A further embodiment of the present invention relates to the functionalized proteins or peptides produced by any of the above methods.

The present invention also provides functionalized proteins or peptides containing a functionalized residue of general formula (IA) as shown below, which can be obtained from the methods described in embodiments (i), (ia) or (ib) of the above described methods.

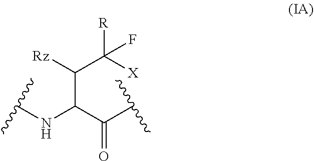

The group Rz represents hydrogen or methyl.

In preferred embodiments Rz represents hydrogen.

R may be as defined in any of the embodiments discussed above.

In particular embodiments, R is the functional group R^{F}; or one or more, preferably one, functional groups R^{F} connected via the linker group L2, wherein R^{F} and L2 are as defined herein. Preferably, R^{F} is a reactive moiety Y selected from: C₂₋₆ alkenyl, C₂₋₆ alkynyl, halogen, —OC(O)R^{a}, —C(O)R^{a}, —CO₂R^{a}, —C(O)(NHNH₂), —ONH₂ and C₁₋₆ azidoalkyl; or R contains a reactive moiety of formula

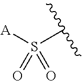

wherein A is as defined above; and wherein the reactive moiety

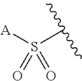

may optionally be connected via a linker group L2.

Preferably, L2 is an alkyl group in which one or more non-adjacent carbon atoms may be optionally substituted for a group selected from NH, O, S, —C(O)NH— or —NHC(O)—. More preferably, L2 is C₁₋₄ alkyl, such as methylene or ethylene.

In further embodiments, R may be a group resulting from the reaction of the functionalized side chain with a further molecule as discussed in “Further reactions of side chains” above.

For instance, R may be a group resulting from the generation of an on-protein radical via, e.g., activation of an on protein ASOOF group followed by reaction with a radical acceptor such as a further protein or peptide containing a SOMO acceptor residue, or a monomer containing a radical acceptor group.

In a particular embodiment R is

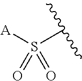

connected either directly or via a linker group, preferably connected directly, wherein A is as defined in relation to the embodiment (i) above.

In a further embodiment R is C₁₋₆ haloalkyl, C₁₋₆ azidoalkyl, or

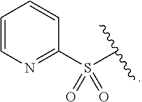 [SMILES: *S(=O)(=O)c1ccccn1]

In a preferred aspect of the above embodiment the group R is

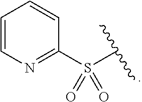 [SMILES: *S(=O)(=O)c1ccccn1]

Such proteins or peptides may be obtained, for instance, via the method of embodiment (ia), i.e. by using an iodo-ASOOF radical precursor compound.

The group X in any of the above definitions may be selected from fluorine or hydrogen. In preferred embodiments X is fluorine.

The present invention further provides functionalized proteins or peptides of general formula (IB) as shown below, which can be obtained from the methods described in embodiments (ii) or (iia) of the above methods.

The functionalized peptides described herein may be obtained by any suitable method as described above.

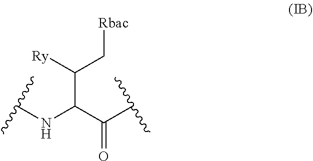

Ry is hydrogen or methyl.

In preferred embodiments Ry represents hydrogen.

Rbac is C₁₋₆ alkyl wherein the terminal carbon is substituted by at least one halogen. In a preferred embodiment, Rbac is C₁₋₄ alkyl wherein the terminal carbon is substituted by at least one halogen. In one aspect of the above embodiments said halogen is bromine or iodine.

In a further embodiment Rbac is represented by the formula below

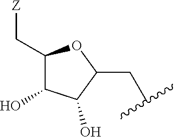

wherein Z is halogen. In one aspect of the above embodiment Z is bromine or iodine.

### Covalently Linking Proteins/Peptides

The functionalized proteins and peptides of the present invention may be further reacted to form covalent bonds with other proteins and peptides, for instance as described in the above section on further reactions of side chains.

Thus, in a further embodiment, the present invention provides a method of covalently linking a functionalized protein or peptide as produced by any of the methods described above, such as those described by formulae (IA) or (IB), in which the group R or Rbac is C₁₋₆ haloalkyl with a further protein or peptide which comprises a group capable of reacting with an alkyl halide to form a covalent bond.

The group capable of reacting with the haloalkyl group may be a suitable nucleophilic group, such as a hydroxyl, thiol, or amine group, such as those found in the side chains of various natural amino acids, such as serine, cysteine, lysine etc. In one preferred embodiment, the group capable of reacting with the haloalkyl group is a thiol groups of a cysteine residue.

In a preferred aspect of the above method, the functionalized protein or peptide and the further protein or peptide are “protein partners” such that they will interact when in solution together, optionally in the presence of further biological molecules such as enzymes and cofactors, to form a protein-protein interface which brings the alkylhalide group into proximity with the group capable of reacting with the alkylhalide group. This proximity allows a reaction between the two groups to take place, e.g. by nucleophilic substitution. This proximity driven reaction greatly increases the effective molarity of the groups with respect to one another, and allows highly site specific covalent binding, as described in examples 5 and 6.

In a particularly preferred embodiment, the protein-protein interface is a binding pocket wherein one of the functionalized protein/peptide and further protein/peptide is held in a binding pocket of the other protein/peptide. In a preferred aspect of this embodiment, at least one of the proteins/peptides is an enzyme and the other is a substrate for said enzyme. The protein or peptide is preferably held in the binding pocket of said enzyme such that the reaction between the alkylhalide group and the group capable of reacting with the alkylhalide group (e.g. nucleophilic group) occurs at the active site of the enzyme. Preferably the active site contains one or more cysteine residues, which are configured to react with the alkylhalide group.

Typically, in the above embodiment, the functionalized protein/peptide is a substrate having an alkyl halide group in a position which will be held in the binding pocket of the further protein/peptide, which is a receptor for the substrate. Where the binding pocket contains a nucleophilic group, in particular a thiol group of a cysteine residue, the alkyl halide in the binding pocket will form a covalent linkage with the cysteine residue.

In a particular aspect of the above embodiment, the enzyme or receptor protein/peptide is inhibited by said binding.

The present invention therefore provides a method for site selectively introducing an alkyl halide group into a protein or peptide such as an enzyme substrate. The alkyl halide group may be introduced in such a position that it enters the active site of the enzyme substrate. For example, a lysine residue which is involved in binding interactions with a substrate may be modified so as to replace it with a DHA residue, which can then be linked to an alkyl halide group using the methods of the present invention. The alkyl halide group so introduced will in turn enter the binding pocket of the substrate and may covalently bond with any nucleophilic group, e.g. a cysteine residue, present in said binding pocket, thereby inactivating the substrate (e.g. an enzyme). In this way, the methods of the present invention may be used to site selectively modify proteins/peptides to provide novel inhibitors.

In a preferred aspect of the above embodiment, the haloalkyl side chain R or Rbac on the functionalized protein or peptide is bromoalkyl or iodoalkyl, preferably C₂₋₃ bromoalkyl or C₂₋₃ iodoalkyl, more preferably —CH₂CH₂BR, —CH₂CH₂I, —CH₂CH₂CH₂BR, or —CH₂CH₂CH₂I.

In a further embodiment the present invention provides a method of covalently linking a functionalized protein or peptide according to formula (IA) above with a further protein or peptide, wherein the group R in the functionalized protein or peptide is

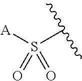

and wherein the further protein or peptide comprises a group capable of reacting with a radical species to form a covalent bond. The group A is as defined above in relation to embodiment (i). The functionalized proteins may be produced by any suitable method, such as those described in embodiment (ia) above.

This covalent bond may be formed via the generation of an on-protein radical as described in the above section on further reactions of side chains, for instance via the application of light in the presence of a suitable photocatalyst and source of Fe(II) as described in detail in the embodiments above, e.g. in embodiment (i) and in example 4. The on-protein radical may then react with the the further protein or peptide which comprises a group capable of reacting with a radical species to form a covalent bond. Such groups capable of reacting with a radical species include SOMO acceptor residues such as alkene groups, e.g. C₁₋₆ alkene groups. Suitable further proteins or peptides are therefore those comprising a residue having aside chain comprising an alkene group, e.g. a C₁₋₆ alkene side chain, preferably dha and or dhb as described above.

In a preferred aspect of this embodiment the further protein or peptide contains one or more dha residues.

In a preferred aspect of the above embodiment, in the functionalized protein of formula (IA), Rz is hydrogen, X is fluorine, and A is heteroaryl. In a more preferred aspect, A is pyridinyl, pyrimidinyl or benzothiazolyl, most preferably 2-pyridinyl.

In a further embodiment the present invention provides a compound of formula (II) or (III) as defined above.

In a still further embodiment, the present invention provides the use of a compound according to formulae (II) or (III) as defined above in a method of functionalizing a protein. In a preferred aspect of said embodiment, said method is one of the methods for protein functionalizing using formula (II) or (III), described above, respectively.

### Definitions

As used herein, the term “alkyl” refers to a linear or branched saturated monovalent hydrocarbon radical having the number of carbon atoms indicated in the prefix. Thus, the term “C₁₋₄ alkyl” refers to a linear saturated monovalent hydrocarbon radical of one to four carbon atoms or a branched saturated monovalent hydrocarbon radical of three or four carbon atoms, e.g. methyl, ethyl, n-propyl, iso-propyl, n-butyl, iso-butyl and tert-butyl. Preferably, an alkyl group is a C₁₋₂₀ alkyl group, more preferably a C₁₋₁₂ alkyl group, yet more preferably a C₁₋₈ alkyl group, and most preferably a C₁₋₄ alkyl group. Derived expressions such as “C₁₋₆ alkoxy”, “C₁₋₆ ester”, “C₁₋₆ azidoalkyl” and “C₁₋₆ ether” are to be construed accordingly.

As used herein, the term “alkenyl” refers to a linear or branched monovalent hydrocarbon radical having the number of carbon atoms indicated in the prefix and containing at least one double bond. Thus, the term “C₂₋₆ alkenyl” refers to a linear monovalent hydrocarbon radical of two to six carbon atoms having at least one double bond, or a branched monovalent hydrocarbon radical of three to six carbon atoms having at least one double bond, e.g. ethenyl, propenyl, 1,3-butadienyl, (CH₂)₂CH═C(CH₃)₂, CH₂CH═CHCH(CH₃)₂, and the like. Preferably, an alkenyl group is a C₂₋₂₀ alkenyl group, more preferably a C₂₋₁₂ alkenyl group, yet more preferably a C₂₋₈ alkenyl group, and most preferably a C₂₋₄ alkenyl group.

As used herein, the term “alkynyl” refers to a linear or branched monovalent hydrocarbon radical having the number of carbon atoms indicated in the prefix and containing at least one triple bond. Thus, the term “C₂₋₆ alkynyl” refers to a linear monovalent hydrocarbon radical of two to six carbon atoms having at least one triple bond, or a branched monovalent hydrocarbon radical of four to six carbon atoms having at least one double bond, e.g. ethynyl, propynyl, and the like. Preferably, an alkynyl group is a C₂₋₂₀ alkynyl group, more preferably a C₂₋₁₂ alkynyl group, yet more preferably a C₂₋₈ alkynyl group, and most preferably a C₂₋₄ alkynyl group.

As used herein, the term “cycloalkyl” refers to a cyclic or bicyclic monovalent hydrocarbon radical having the number of carbon atoms indicated in the prefix. A cycloalkyl group is typically saturated. Thus, the term “C₃₋₁₀ cycloalkyl” may refer to, e.g. cyclopropyl, cyclobutyl, cyclopentyl, or cyclohexyl, and the like; or to bicyclo[3.1.0]hexanyl, bicyclo[4.1.0]heptanyl and bicyclo[2.2.2]octanyl and the like.

As used herein, the term “heterocyclyl” refers to a monovalent monocyclic or bicyclic group of 4 to 8 ring atoms in which one or two ring atoms are heteroatoms selected from N, O, or S(O)n, where n is an integer from 0 to 2, the remaining ring atoms being C. The term heterocyclyl includes, but is not limited to, pyrrolidinyl, piperidinyl, homopiperidinyl, morpholinyl, piperazinyl, tetrahydropyranyl, thiomorpholinyl, and the like.

As used herein, the term “aryl” refers to a monovalent monocyclic or bicyclic aromatic hydrocarbon radical of 6 to 10 ring atoms, e.g. phenyl or naphthyl, and the like.

As used herein, the term “heteroaryl” refers to a monovalent monocyclic or bicyclic aromatic radical of 5 to 10 ring atoms where one or more, preferably one, two, or three, ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like, preferably pyridinyl, pyrimidinyl, pyrazinyl, or pyridazinyl.

As used herein, the term “alkoxy” refers to an —OR⁹ radical where R⁹ is alkyl as defined above, e.g., methoxy, ethoxy, n-propoxy, iso-propoxy, n-butoxy, iso-butoxy, tert-butoxy and the like. Preferably, an alkoxy group is a C₁₋₂₀ alkoxy group, more preferably a C₁₋₁₂ alkoxy group, yet more preferably a C₁₋₈ alkoxy group, and most preferably a C₁₋₄ alkoxy group.

As used herein, the term “halo” refers to fluoro, chloro, bromo, or iodo, preferably fluoro or chloro.

As used herein, the term poly(ethyleneglycol) refers to a divalent radical polymer of formula

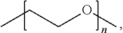

n is not particular limited, but may be from 1 to 500, preferably 1 to 200, more preferably 1 to 50 and wherein one end is covalently bonded to a group, such as the functionalized protein or peptide and the other end is bonded to a hydrogen atom, or to a further group. In some embodiments n is from 1 to 10, typically 1 to 5, preferably 1 to 3.

As used herein the term photocatalyst refers to a redox catalyst which increases its oxidative and/or reductive potential in response to stimulation by light radiation of an appropriate flux, e.g. due to excitation of an electron to a higher energy level. Oxidative half potentials as defined herein are measured against a saturated calomel electrode. The oxidative half potential of the photocatalyst is its oxidative half potential of the catalyst in its oxidized state, typically its photo-activated state.

Where the compounds and functional groups described herein have one or more asymmetric centres, they may accordingly exist as enantiomers. Where the compounds of use in the invention possess two or more asymmetric centres, they may additionally exist as diastereomers. The invention is to be understood to extend to the use of all such enantiomers and diastereomers, and to mixtures thereof in any proportion, including racemates. The formulae depicted hereinafter are intended to represent all individual stereoisomers and all possible mixtures thereof, unless stated or shown otherwise. In addition, some of the compounds and groups described herein may exist as tautomers, for example keto (CH₂C═O)↔enol (CH═CHOH) tautomers or amide (NHC═O)↔hydroxyimine (N═COH) tautomers. The formulae depicted hereinafter are intended to represent all individual tautomers and all possible mixtures thereof, unless stated or shown otherwise.

Unless otherwise specified, it is to be understood that each individual atom present in the groups or formulae defined herein, may in fact be present in the form of any of its naturally occurring isotopes, with the most abundant isotope(s) being preferred. Thus, by way of example, each individual hydrogen atom present in the formulae defined herein, may be present as a ¹H, ²H (deuterium) or ³H (tritium) atom, preferably ¹H. Similarly, by way of example, each individual carbon atom present in any of the formulae depicted herein, may be present as a ¹²C, ¹³C or ¹⁴C atom, preferably ¹²C.

Where the compounds of use in the invention carry an acidic moiety, e.g. carboxy, the present disclosure also covers suitable salts thereof, such as alkali metal salts, e.g. sodium or potassium salts; alkaline earth metal salts, e.g. calcium or magnesium salts; ammonium salts; and salts formed with suitable organic ligands, e.g. quaternary ammonium salts.

When a moiety is said to be optionally substituted it may be substituted by, for example 0, 1, 2 or 3 groups. In some embodiments it is substituted by 0, 1 or 2, groups, preferably 0 or 1 groups.

When groups are attached to another group, e.g. wherein a peptide, pharmaceutical drug or sugar is bonded to a linker they may be attached via any suitable means known to the person skilled person in the field of protein conjugation, such as through esterification with a hydroxyl group or carboxy group on the molecule of interest.

As used herein, the term “amino acid” refers to any natural or synthetic amino acid, that is, an organic compound comprising carbon, hydrogen, oxygen and nitrogen atoms, and comprising both amino (—NH₂) and carboxylic acid (—COOH) functional groups. Typically, the amino acid is an α-, β-, γ- or δ-amino acid. Preferably, the amino acid is one of the twenty-two naturally occurring proteinogenic α-amino acids. Alternatively, the amino acid is a synthetic amino acid, for example selected from α-Amino-n-butyric acid, Norvaline, Norleucine, Alloisoleucine, t-leucine, α-Amino-n-heptanoic acid, Pipecolic acid, α,β-diaminopropionic acid, α,γ-diaminobutyric acid, Ornithine, Allothreonine, Homocysteine, Homoserine, β-Alanine, β-Amino-n-butyric acid, β-Aminoisobutyric acid, γ-Aminobutyric acid, α-Aminoisobutyric acid, isovaline, Sarcosine, N-ethyl glycine, N-propyl glycine, N-isopropyl glycine, N-methyl alanine, N-ethyl alanine, N-methyl β-alanine, N-ethyl β-alanine, isoserine, α-hydroxy-γ-aminobutyric acid, Homonorleucine, O-methyl-homoserine, O-ethyl-homoserine, selenohomocysteine, selenomethionine, selenoethionine, Carboxyglutamic acid, Hydroxyproline, Hypusine, Pyroglutamic acid, aminoisobutyric acid, dehydroalanine, β-alanine, γ-Aminobutyric acid, δ-Aminolevulinic acid, 4-Aminobenzoic acid, citrulline, 2,3-diaminopropanoic acid and 3-aminopropanoic acid. Further, the amino acid may be dehydroalanine, dehydrobutyrine, or a synthetic dehydroalanine or dehydrobutyrine precursor. An amino acid which possess a stereogenic centre may be present as a single enantiomer or as a mixture of enantiomers (e.g. a racemic mixture). Preferably, if the amino acid is an α-amino acid, the amino acid has L stereochemistry about the α-carbon stereogenic centre.

All documents referenced herein are hereby incorporated by reference.

### Examples

The following are examples that illustrate the present invention. However, these examples are in no way intended to limit the scope of the invention.

Unless specified otherwise, parameters and values are measured as set out in the following examples.

### General Methods

Unless otherwise noted, chemical reagents, media, and Escherichia coli cell stocks were obtained from commercial suppliers (Sigma-Aldrich, Fluorochem, Carbosynth, VWR, Alfa Aesar, Fisher Scientific) and used without further purification. Sonication was performed using a Fisher Scientific Model 505 Sonic Dismembrator. Proteins were purified using an Äkta FPLC System UPC-900 (GE Healthcare, UK). Gel electrophoresis was performed using Invitrogen NuPAGE 4-12% Bis-Tris gels, Novex MiniCell tanks, and a BioRad PowerPac controller. Western blotting was performed using an iBlot gel transfer device from Thermo-Fisher. Antibodies were used as per the manufacturer's recommendations: anti-Histone H3 (96C10) Mouse mAb for histone detection, Mouse monoclonal Anti-polyHistidine-Alkaline Phosphatase, Clone HIS-1 (Sigma, A5588) for KDM4A detection (6His tag), Rabbit Anti-Mouse IgG (H+L) HRP conjugate (Promega, W4021) and Goat Anti-Mouse IgG H&L Alkaline Phosphatase (Abcam, ab97020) as secondary antibodies. Thin layer chromatography was performed using Silica Gel 60 F254 plates (Merck) using 1-10% methanol in dichloromethane. Nuclear magnetic resonance spectra were recorded on a Bruker AVIII HD 400 nanobay (400 MHz) spectrometer and analyzed on MestReNoval 1. Carbon nuclear magnetic resonance spectra were recorded on a Bruker DQX 400 (100 MHz) spectrometer. All 1H-NMR chemical shifts are quoted in ppm using residual solvent as the internal standard relative to TMS (d6-acetone: 2.09 ppm). All 13C NMR chemical shifts are quoted in ppm using the central solvent peak as the internal standard relative to TMS (d6-DMSO 39.3 ppm). Coupling constants (J) are reported in Hertz (Hz). Infrared (IR) spectra were recorded on a Bruker Tensor 27 Fourier-Transform spectrophotometer. High-resolution small molecule mass spectra were recorded on a Micromass LCT (resolution=5000 RWHM) using a lock-spray source. Protein crystal structures were analyzed and displayed using MacPyMOL v. 1.3 (Schrodinger, Inc.). Synthetic gene fragments (i.e. for human histone eH3-FLAG-HA constructs) were obtained from GeneArt Gene Synthesis (Thermo-Fisher). Nucleotide sequences were confirmed by the Source Bioscience DNA Sanger sequencing services based at Oxford University.

### Mass Spectrometry

Liquid Chromatography-Mass Spectrometry/Mass Spectrometry (LC-MS/MS) were used to confirm site-selective post-transcriptional protein editing and to identify possible side products. The general workflow for bottom-up LC-MS/MS analysis of post-translationally edited proteins is desribed below. Samples were reduced (commonly with TCEP or DTT) and alkylated (with iodo- or chloroacetamide). Proteins were digested with a protease (Trypsin, ArgC, LysC, AspN, Elastase etc.) and resulting peptides analysed. Proteomics software such as PEAKS can perform de-novo sequencing on the measured spectra or compare these to a database of protein sequences. Modifications were identified and manually validated.

### Intact Protein Mass Spectrometry

Intact protein mass spectrometry was performed on a Waters Xevo G2-S QTof coupled to Water Acquity UPLC. Separation was achieved using a Thermo Proswift (250 mm×4.6 mm×5 m) column with water+0.1% formic acid (solvent A) and acetonitrile+0.1% formic acid (solvent B) as the eluent system over a 10-minute linear gradient. Nitrogen was used as the desolvation gas (600 L/h) for positive electrospray ionization. Voltages used were capillary: 3000 V, cone: 160 V. Lock-spray analysis ensured continual calibration against a leucine enkephalin standard solution.

Raw spectra containing multiple charged ion series were deconvoluted using MassLynx (Waters) and its maximum entropy (MaxEntl) deconvolution algorithm (Resolution: 1.00 Da/channel, Width at half height: ion series/protein dependent, Minimun intensity ratios: 33% Left and Right). Spectra were deconvoluted between 10000 and 20000 Da for Xenopus laevis Histone H3, between 10000 and 25000 Da for human Histone eH3.1, between 5000 and 15000 Da for Xenopus laevis Histone H4, between 10000 and 30000 Da for NPP, between 30000 and 50000 Da for AcrA, and between 30000 and 40000 Da for PanC. Any reaction conversions were calculated from relative peak intensities in the deconvoluted spectra. On histones, ˜10% baseline methionine oxidation often occurred during production, storage, and use, and these “+16 Da adducts” were combined into this total sums for starting material/products.

### Tandem Mass Spectrometry

### ArgC In-Solution Digest

### Variant 1: Denatured Protein Samples, No Alkylation

Approx. 10 μg (20 μL) of desalted & denatured modified protein sample were taken in 50 mM TEAB to a total volume of 100 μL and reduced with 10 mM TCEP for 30 min at r.t. Samples were digested with Arg-C(1:20 w/w) in activation buffer (50 mM TEAB, 0.2 mM EDTA, 5 mM TCEP) for 3 h at 37° C. The reaction was stopped by addition of 10% FA to a final concentration of 0.5%. Samples were desalted by C18 (Oasis HLB 10 mg) and dried in a speed-vac before being resuspended in 5% FA 5% DMSO.

Variant 2: With Denaturation, with Alkylation

Approx. 10 μg of modified protein sample were taken in 8M urea in 50 mM TEAB containing 20 mM methylamine to a total volume of 100 μL and denatured for 30 min at room temperature. Samples were reduced with 10 mM TCEP for 30 min at room temperature and alkylated with 50 mM chloroacetamide for 30 min at room temperature in the dark. Samples were diluted to 1M urea and digested with Arg-C(1:20 w/w) in activation buffer (50 mM TEAB, 0.2 mM EDTA, 5 mM TCEP) for 4 h—O/N at 37° C.

The reaction was stopped by addition of 10% FA to a final concentration of 0.5%. Samples were desalted by C18 (Oasis HLB 10 mg cartridge) and dried in a speed-vac before being resuspended in 5% FA 5% DMSO.

### LysC In-Solution Digest

Approx. 10 μg (20 μL) of desalted & denatured modified protein sample were taken in 8 M urea in 100 mM TEAB to a total volume of 100 μL. Samples were reduced with 10 mM TCEP for 30 min at room temperature and alkylated with 50 mM chloroacetamide for 30 min at room temperature in the dark. The solution was diluted to 6M urea with 50 mM TEAB and digested with LysC 1:20 (w/w) over night at 37° C. %. Samples were desalted by C18 (Oasis HLB 10 mg) and dried in a speed-vac before being resuspended in 5% FA 5% DMSO.

### Tryptic or AspN or Elastase In-Solution Digest

Approx. 10 μg (20 μL) of desalted & denatured modified protein sample were taken in 8 M urea in 100 mM TEAB to a total volume of 100 μL. Samples were reduced with 10 mM TCEP for 30 min at room temperature and alkylated with 50 mM chloroacetamide for 30 min at room temperature in the dark. The solution was diluted to 1 M urea with 50 mM TEAB and digested with AspN or Trypsin or Elastase 1:20 (w/w) 4 h to overnight at 37° C. %. Samples were desalted by C18 (Oasis HLB 10 mg) and dried in a speed-vac before being resuspended in 5% FA 5% DMSO.

### Data Acquisition

### Standard Data Acquisition—Q Exactive

Resulting peptides were separated by nano-flow reversed-phase liquid chromatography Ultimate 3000 UHPLC system (Thermo Fisher Scientific) coupled to a Q Exactive Hybrid Quadrupole-Orbitrap mass spectrometer (Thermo Fischer Scientific). The peptides were loaded on a C18 PepMap100 precolumn (inner diameter 300 m×5 mm, 3 m C18 beads; Thermo Fisher Scientific) and separated on an in-house packed analytical column (75 m inner diameter ×50 cm packed with ReproSil-Pur 120 C18-AQ, 1.9 m, 120 Å, Dr. Maisch GmbH). Separation of cross-linked peptides was conducted with a first step linear gradient from 15 to 35% of B for 30 min followed by a second step from 35% to 55% of B for additional 15 min, at a flow rate of 200 nl/min (A: 0.1% formic acid, B: 0.1% formic acid in acetonitrile). The raw data were acquired on the mass spectrometer in data-dependent mode. Automatically switching from MS to higher energy collision induced dissociation MS/MS. Full-scan spectra were acquired in the Orbitrap [scan range 350-2000 m/z, resolution 70000, automatic gain control (AGC) target 3×106, maximum injection time 50 ms]. After the MS scan, the top 10 most intense peaks were selected for HCD fragmentation at 30% of normalised collision energy. HCD spectra were also acquired in the Orbitrap (resolution 17500; AGC target 5×104; maximum injection time 120 ms), with first fixed mass at 180 m/z.

### Data Acquisition for Crosslinked Samples—Q Exactive

Full-scan spectra were acquired in the Orbitrap [scan range 350-2000 m/z, resolution 70000, automatic gain control (AGC) target 3×106, maximum injection time 100 ms]. after the MS scan, the top 10 most intense peaks were selected for HCD fragmentation at 30% of normalised collision energy, excluding 1+ and 2+ charged species. HCD spectra were also acquired in the Orbitrap (resolution 17500; AGC target 5×104; maximum injection time 120 ms, scan range 200-2000 m/z), with first fixed mass at 180 m/z.

Data analysis (tandem mass spectrometry) Standard data analysis Searches were performed with Peaks version 8.5 (Bioinformatics Solutions Inc.) for identification and de-novo analysis. The raw MS file was searched against the given protein sequence and a list of contaminants (generated from MaxQuant contaminants database). Samples were additionally searched with MaxQuant against the UniProt human database for confirming the purity of the sample. Precursor mass tolerance was set to 10 ppm. Fragment mass tolerances for HCD was set to 0.02 Da. The corresponding protease was selected with a maximum number of 3 missed cleavages and non-specific cleavage at one end of the peptide. For Elastase, an unspecific search was used. Oxidation (Methionine), Deamination (Asparagine, Glutamine), Carbamidomethylation (Cysteine —except for ArgC variant 1 digest), Carbamylation (lysine, peptide N-term), Amidation (C-terminus) and dehydroalanine (Cysteine, −33.9887) were set as variable modifications, as well as the sample-specific modifications in the following table below. A maximum of 4 variable modifications was set. A FDR of 1% on peptide level and de-novo ALC of 80 was applied. All spectra and identifications were manually validated. For analysis of isotopic pattern, manual analysis with XCalibur Qual Browser 4.0 was performed.

### Crosslinking Mass Spectrometry Analysis

Crosslinked samples were searched with Peaks 8.5 as described above to confirm presence of both proteins and to confirm sample purity. Crosslinked samples were processed with the pLink 2.3.5 software package. Using the pConfig module, the amino acid ‘B’ of mass 205.01023 and composition H(12)C(7)N(1)O(1)Br(1) was defined. Note that the isotopic pattern doesn't matter for crosslinking analysis as HBr gets eliminated. The linker 4BrBut was defined as linking the alpha amino acid B to the beta amino acids STYCHRKWDENQ with the linker composition H(−1)Br(−1).

First search mass tolerance was set to 20 ppm and fragment mass tolerances at 20 ppm. A mass filter of 10 ppm was applied. E-values were computed and a global FDR of 1% set. Trypsin (or LysC for H3-K4) was selected as a protease with a maximum number of 3 missed cleavages. Carbamidomethylation (Cysteine), Oxidation (Methionine), Deamidation (Asparagine, Glutamine), Carbamylation (lysine, peptide N-term), Amidation (C-terminus) were set as variable modifications. RAW files were searched against a database containing the sequence of the modified eH3 construct and the expressed KDM4. Resulting spectra were manually analysed and validated using pLabel 2.3.5. As an empirical rule, an e-value higher than e-03 indicates a potential identification, higher than e-06 a reliable identification and higher than e-10 a very good identification.

### Cyclic Voltammetry

Catechol and 4-bromobutylboronic acid were obtained from Sigma-Aldrich and used as received without further purification. All solutions were made up using ultrapure water of resistivity not less than 18.2 MQ cm (Millipore) at 25° C. and degassed thoroughly with nitrogen (99.998%, BOC Gases plc) before use. Phosphate buffered saline (PBS) solution (pH=6.0) consisting of 43.85 mM sodium phosphate monobasic and 6.15 mM potassium phosphate dibasic.

All voltammetric measurements were recorded using an Autolab PGSTAT30 computer controlled potentiostat (Metrohm, Utrecht, The Netherlands). Experiments were performed in a thermostatted (25.0+0.3° C.) Faraday cage using a three-electrode set-up. A glassy carbon macroelectrode (diameter 3.0 mm, CH Instrument) was used as the working electrode, a saturated calomel electrode (SCE) as the reference electrode (SCE, ALS distributed by BASi, Tokyo, Japan) and a graphite rod as the counter electrode. Prior to each voltammetric experiment, renewal of the working electrode surface was achieved by polishing with alumina slurries in the size sequence 1.0 m, 0.3 m and 0.05 m, (Buehler Ltd, USA) followed by sonication in water and drying with nitrogen.

### HPLC Analysis and Comparison to LC-MS Results

All BACED and pySOOF reactions performed were monitored via LC-MS analysis of the crude reaction product, where the chromatogram was constructed based on total ion count detect by the mass spectrometer. In these cases, the ion series was produced by combining all spectra contained within the time encompassed by the protein peak in the chromatogram (see below, protein peak maximas usually occurred around 4.50 minutes).

HPLC analysis was performed on a Shimadzu 2020 LC-MS instrument with an LCM20AD pump, SPD-20A UV/Vis operating at 220 nm and 280 nm using a Phenomenex Jupiter C-4 (5 mm, 300 Å) 4.6×250 mm column operating at a flow rate of 1 mLmin-1. The analysis was performed using a mobile phase of 0.1 vol % TFA in water (Solvent A) and 0.1 vol % TFA in MeCN (Solvent B) using a linear gradient as follows: 0% B for 4 min, 0 to 100% B over 26 min. Chromatograms recorded at 220 nm were analysed using the Shimadzu LabSolutions software.

### ¹⁹F-NMR Studies

¹⁹F-NMR studies were performed according to the general procedure below: A glass vial (5 mL) was charged with FeSO₄·7H₂O (100 eq) and transferred into a glovebox. Then, an aliquot of Dha-tagged protein (1.5-4.6 mg, 0.5-1 mL, typical protein concentration of 3-4.6 mg/mL), pySOOF-reagent (5 eq in DMSO [1M]) and Ru(bpy)₃Cl₂ (2.5 eq in 10 μL water) were added to the glass vial. Afterwards, the vial was sealed with a plastic cap, transferred out of the glovebox and irradiated with blue LED light (50 W) for 15 minutes. The crude reaction mixture was treated with either DTT (10 mg) or EDTA (5 mg), vortexed for 30 s and purified by PD MiniTrap G-25 column (GE Healthcare) followed by a PD MidiTrap G-25 column (GE Healthcare) (both column equilibrated with buffer in D₂O following gravity protocol) yielding a fluorine-labelled protein. After concentration of the protein sample to a volume of 0.5 mL using a vivaspin column (MCW=5000) the sample is ready for recording a ¹⁹F-NMR spectra.

### Histone Formation

Bacterial expression plasmids encoding all canonical Xenopus laevis histones in a pET3 production vector was used. The gene for the WT human histone eH3.1 (C-terminal FLAG-HA tag, C96A and C110A)24,25 was obtained from Thermofischer (GeneArt service) and cloned into the pET3d expression plasmid at the NcoI and BamHI restriction enzyme sites. Quickchange mutagenesis was performed per the manufacturer's instructions (QuikChange II Site-Directed Mutagenesis Kit, Agilent) to create the desired cysteine mutants. The Escherichia coli strain BL21(DE3)pLysS was transformed as appropriate and selected on chloramphenicol and ampicillin. Single colonies were used to inoculate 5-20 mL starter cultures in LB broth with the same antibiotics. Flasks containing 500 mL 2×TY media were inoculated with 1% v/v of starters, grown at 37° C. until OD600=0.4-0.8. The production of histones were induced with 0.5 mM IPTG and allowed to proceed for 2 h before harvest and resuspension into 5-fold volume/weight “wash buffer” (50 mM Tris, pH 7.5, 100 mM NaCl with a protease inhibitor cocktail). Suspensions were flash-frozen and stored at −80° C. until lysis. Lysis proceeded via sonication in the presence of 1 mg DNase for 5×30 second bursts at 40% amplitude. The sonicate was centrifuged for 20 min at 20 krpm at 4° C. The supernatant was discarded and the pellet resuspended in 40 mL [“wash buffer”+1% Triton-X detergent]. Sonication was repeated once at 40% amplitude, 30 seconds, and the suspension centrifuged at 20 krpm for 10 minutes. The pellet was washed twice more in this fashion, then once with the non-Triton containing “wash buffer.” 1 mL of DMSO was added to the pellet and crudely mixed with a spatula to aid histone desolvation for 10 minutes. 10 mL “unfolding buffer” (7M Gdn·HCl, 20 mM Tris, pH 7.5, 10 mM DTT) was added and shaken for 1 h at rt, then the mixture was centrifuged for 10 minutes at 20 krpm at room temperature. The supernatant was loaded onto an S200 size exclusion column (GE Healthcare) pre-equilibrated with “SAU-100” buffer (7M urea, 20 mM NaOAc, pH 5.2, 100 mM NaCl, 1 mM EDTA, 10 mM DTT, 1 mM benzamidine). Protein was eluted with SAU-100, analyzed by SDS-PAGE, and histone fractions were pooled and concentrated to 1-4 mL. Cation exchange chromatography was used to further purify histones (HiTrap SP 5 mL) using a linear gradient of 0-100% SAU-1000 buffer (“SAU-100” with 1000 mM NaCl final concentration). Pure fractions were pooled, dialyzed against water (with 2 mM β-mercaptoethanol) and lyophilized.

### AcrA

Plasmids (pET24) were transformed into BL-21(DE3) cells and plated on Kanamycin agar plates. Four 10 mL starter cultures (LB/Kanamycin) of each plasmid were grown over night as 37° C. then transferred into 500 mL of media (LB/Kanamycin). The cultures were grown at 37° C. until OD600=0.6-0.8 (all between 40-70 min) at which point the cells were induced with IPTG (1.25 mM) and incubated for a further 4 h. The cells were then pelleted for 10 minutes at 8 krpm. Cell pellets were resuspended in buffer (50 mL of 50 mM Tris, 100 mM NaCl, 10 mM imidazole, 1 mg/mL lysozyme and 0.1 mg/mL DNAse) and stirred on ice for 2 h. The pellets were then subjected to sonification (50% power, 30 s sonication 1 min rest, four times), with the resultant mixtures treated by centrifugation (20 krpm, 45 min). The supernatant was purified using Ni-NTA resin (50 mL of 50 mM tris, 100 mM NaCl, 5 mM imidazole binding buffer and 20 mL of 50 mM Tris, 100 mM NaCl, 250 mM imidazole elution buffer). The fractions containing the desired protein (visualised by SDS-PAGE analysis) were then dialysed into 20 mL of 50 mM Tris, 100 mM NaCl and the concentration analysed.

### Human Sirtuin 2 (Sirt2)

The gene for Human Sirt2 in pET6 plasmid was transformed in BL21-(DE3) cells and plated on LB/agar/carbenicillin plates. Single colonies were picked into 5 mL of LB/carbenicillin and grown at 37° C. 250 RPM overnight. The starter culture was poured into 4×500 mL of super broth media and grown to an OD of 0.6 at 37° C. 250 RPM (2 hours). Expression was induced by the addition of IPTG stock to give the final concentration of 0.3 mM then incubated at 37° C. 250 RPM for 4 h. Cells were pelleted at 8 kRPM 9.6 kG at raverage for 15 minutes then the pellets frozen at −80° C. Pellets were thawed on ice then resuspended in buffer (NaCl 500 mM, Tris 50 mM, Glycerol 5%, PME 5 mM, Imidazole 25 mM and one Roche cOmplete EDTA-free protease inhibitor cocktail tablet at a pH of 7.5, 10 mL). Cells were lysed by sonication on ice (30% Amplitude, for 5 minutes of 2 s on 2 s off). The insoluble fraction was removed by centrifugation (25 kRPM 52 kG at average for 1 h) and the lysate filtered through a 0.2 m syringe filter before being applied to a FPLC column. The protein was purified by 2D-FPLC firstly through a 1 mL ff-Histrap (A: NaCl 500 mM, Tris 50 mM, Glycerol 5%, PME 5 mM, Imidazole 25 mM pH of 7.5, B: A+225 mM Imidazole pH 7.5, 5 CV A 10 CV B step gradient). Fractions containing the desired protein (analysed by SDS-PAGE) were concentrated to −5 mL using a 10 kDa GE vivaspin then passed through an s200 36/60 sec column in 150 mM NaCl, 25 mM Tris pH 8.0 buffer to give 50 mL of 0.1 mg/mL protein.

Expression and purification was carried out for the following proteins as set out in the rereferences below

### PanC:

Dadová, J. et al. Precise Probing of Residue Roles by Post-Translational β,γ-C,N Aza-Michael Mutagenesis in Enzyme Active Sites. ACS Central Science 3, 1168-1173, doi:10.1021/acscentsci.7b00341 (2017)
   cabLys3:
Chen, Z.-L. et al. A high-speed search engine pLink 2 with systematic evaluation for proteome-scale identification of cross-linked peptides. Nature Communications 10, 3404, doi:10.1038/s41467-019-11337-z (2019).

### NPP-G2F-C61:

Wright, T. H. et al. Posttranslational mutagenesis: A chemical strategy for exploring protein side-chain diversity. Science, aag1465, doi:10.1126/science.aag1465 (2016).

### Dehydroalanine Formation

X.I. Histone H3-Dha9—Lyophilized X.I. Histone H3-C9 (10 mg) was added to denaturing phosphate buffer (100 mM NaPi, pH 8, 3 M Gdn·HCl, 500 μL) and mixed until fully dissolved. DTT was added (30 mg) and shaken for 30 min at rt, 500 rpm, to reduce disulfide bonds, before being removed via desalting into 1 mL of the same buffer (PD Minitrap G25, GE Healthcare). The resulting protein concentration was determined (Nanodrop) and immediately followed by the addition of DBHDA (60 eq from a freshly prepared 0.5 M DMSO stock) and subsequent shaking (500 rpm) at 25° C. for 45 min, then 37° C. for 2 h. The protein was desalted as before to remove the excess DBHDA and exchange into the desired buffer. Protein yield and concentration was determined by Nanodrop and conversion was determined by LC-MS analysis.

Corresponding procedures were used for formation of Human histone eH3.1-Dha4 and Human histone eH3.1-Dha9 from Human histone eH3-C4, and Human histone eH3-C4, respectively.

AcrA-Dha123 formation was carried out as described in Wright, T. H. et al. Posttranslational mutagenesis: A chemical strategy for exploring protein side-chain diversity. Science, aag1465, doi:10.1126/science.aag1465 (2016).

PanC-Cys44/47—PanC-Cys44/47 was buffer exchanged from storage buffer to sodium phosphate buffer (100 mM, pH 8.0, 3M Gdn·HCl) using PD MiniTrap G-25 column (GE Healthcare) equilibrated with sodium phosphate buffer (100 mM, pH 8.0, 3M Gdn·HCl) following gravity protocol to give a protein solution with a concentration of 2.56 mg/mL. An 1 mL aliquot (29.2 nmol) was treated with methyl 2,5-dibromopentanoate (MDBP, 1M in DMSO, 1.46 μmol) and shaken at 25° C. with 500 rpm for 16 hours. Then, the excess of alkylation reagent was removed using PD MidiTrap G-25 column (GE Healthcare) equilibrated with ammonium acetate buffer (500 mM, pH 6.0, 3M Gdn·HCl) following gravity protocol to give a protein solution with a concentration of 1.56 mg/mL. Conversion was determined by analysis of an aliquot of the purified product by LC-MS.

cabLys3-Dha104—cabLys3-Dha104An aliquot of cabLys3-Cys 104 in PBS buffer (pH 7.4) was treated with DTT (4 mg) and incubated for 30 min at 25° C. Afterwards, DTT was removed using PD MidiTrap G-25 column (GE Healthcare) equilibrated with sodium phosphate buffer (50 mM, pH 8.0) following gravity protocol to give a crude protein solution with a concentration of 0.9 mg/mL (0.5 mL) after concentration using a vivaspin column (MCW=5000). Then, DBHDA (0.5M in DMSO, 14.25 μmol) was added to the protein solution and the resulting reaction mixture was incubated for 150 min at 37° C., followed by a purification step by PD MiniTrap G-25 column (GE Healthcare) equilibrated with ammonium acetate buffer (100 mM, pH 6.0) following gravity protocol. After concentration of the protein sample using a vivaspin column (MCW=5000), a 0.5 mL stock solution of Dha-tagged cabLys3 was obtained with a protein concentration of 0.9 mg/mL.

### Synthesis Examples

The reagents and compounds used in the examples were synthesized according to the following methods.

¹H NMR and ¹³C NMR data for these compounds were obtained and confirmed against literature values.

### 1-Allyl-2,3,5-tri-O-benzoyl-α-D-ribofuranose

1-O-Acetyl-2,3,5-tri-O-benzoyl-β-D-ribofuranose (10.0 g, 19.8 mmol) was added to an ice-cold mixture of allyltrimethylsilane (9.45 mL, 59.5 mmol) in 200 mL acetonitrile followed by dropwise addition of BF₃·OEt₂ (2.69 mL, 21.8 mmol). The reaction mixture was allowed to warm to rt over a 4 h period, then diluted with aqueous saturated NaHCO₃ solution and extracted with Et₂O. The combined organic layer was dried over MgSO₄, filtered and concentrated. The oily residue was purified by column chromatography (SiO₂, pentane:ethyl acetate (8:2)) to give 1-allyl-2,3,5-tri-O-benzoyl-α-D-ribofuranose (7.22 g, 14.9 mmol, 75%) as a green oil.

C₂₉H₂₆O₇ (486.5 g/mol).

### 1-Allyl-α-D-ribofuranose

NaOH (3.09 g, 57.2 mmol) added to a stirred solution of 1-allyl-2,3,5-tri-O-benzoyl-α-D-ribofuranose (7.00 g, 14.3 mmol) in 50 mL MeOH under N2. The resulting reaction mixture was stirred for 1 hour, then cooled to 0° C. and carefully neutralized with a methanolic solution of HCl (ca. 1M). The crude mixture was concentrated under reduced pressure and purified by column chromatography (SiO₂, ethyl acetate) to give 1-allyl-α-D-ribofuranose (2.10 g, 12.1 mmol, 85%) as a yellow oil.

C₈H₁₄O₄ (174.2 g/mol).

### 1-Allyl-2,3-isopropylidene-α-D-ribofuranose

1-Allyl-α-D-ribofuranose (2.00 g, 11.5 mmol) was added to a solution of p-toluenesulfonic acid monohydrate (9.72 g, 51.1 mmol) and triethyl orthoformate (12.1 mL, 72.6 mmol) in 200 mL acetone. The reaction mixture was stirred at rt overnight. After neutralisation with saturated aqueous Na₂CO₃ solution, the crude mixture was concentrated to a small volume of MeOH to crystallize the product out of solution at 0° C. The resulting product was filtrated to give 1-allyl-2,3-isopropylidene-α-D-ribofuranose (1.50 g, 7.01 mmol, 60%) as a white solid.

C9H₁₈BN₃O₂ (211.1 g/mol).

### 1-Allyl-2,3-isopropylidene-5-bromo-α-D-ribofuranose

Under inert atmosphere, CBr₄ (1.55 g, 4.67 mmol) and polymer bound PPh₃ (1.23 g, 4.68 mmol) were added to a solution of 1-allyl-2,3-isopropylidene-α-D-ribofuranose (0.50 g, 2.34 mmol) in dry CH₂Cl₂ (10 mL) at 0° C. The resulting reaction mixture was stirred overnight at room temperature. Then, the resin was filtered off and the organic layer was washed with water (2×10 mL), dried over Na₂SO₄, filtered off and evaporated to dryness. The crude product was purified by column chromatography (SiO₂, pentane:ethyl acetate (9:1)) to give 1-allyl-2,3-isopropylidene-5-bromo-α-D-ribofuranose (0.34 g, 1.17 mmol, 50%) as a yellow oil.

C₁₁H₁₇BrO₃ (277.2 g/mol).

### 1-Allyl-2,3-isopropylidene-5-chloro-α-D-ribofuranose

Under inert atmosphere, 1-allyl-2,3-isopropylidene-α-D-ribofuranose (0.20 g, 0.93 mmol) and polymer bound PPh₃ (0.49 g, 1.87 mmol) were dissolved in CCl₄ (10 mL) followed by addition of imidazole (3 mg, 0.05 mmol) and the resulting reaction mixture was heated to reflux overnight. Then, the reaction was quenched by the addition of ice-cold water, diluted with CH₂Cl₂ and filtered over celite. After evaporation of the solvent under reduced pressure, the crude product was purified by column chromatography (SiO₂, pentane:ethyl acetate (9:1)) to give 1-allyl-2,3-isopropylidene-5-chloro-α-D-ribofuranose (0.17 g, 0.74 mmol, 78%) as a yellow oil.

C₁₁H₁₇ClO₃ (232.7 g/mol).

### (4-(5-Bromo-α-D-ribofuranose)butyl)boronic acid

Under inert atmosphere, BCl₃ in CH₂Cl₂ (1M, 0.71 mL, 0.71 mmol) was carefully added to a mixture of 1-allyl-2,3-isopropylidene-5-bromo-α-D-ribofuranose (0.13 g, 0.48 mmol) and SiEt₃H (91.3 μL, 0.57 mmol) at −78° C. The resulting suspension was stirred at this temperature for 30 min, after which it was allowed to warm to rt overnight. The HCl generated in situ induced the deprotection of the acetonide groups. The resulting mixture was diluted with water and Et₂O and the aqueous layer was extracted with Et₂O. The combined organic layers were washed with brine and dried over MgSO₄. After removal of the solvent under reduced pressure, the crude product was purified by Prep HPLC using a RP XBridge Prep C18 column with a mobile phase of 0.25% NH₄CO₃ solution in Water: CH₃CN to give (4-(5-bromo-α-D-ribofuranose)butyl)boronic acid (90.0 mg, 0.32 mmol, 67%) as a white solid.

C₈H₁₆BBrO₅ (282.9 g/mol).

### (4-(5-Chloro-α-D-ribofuranose)butyl)boronic acid

Under argon atmosphere, BCl₃ in CH₂Cl₂ (1 M, 0.85 mL, 0.85 mmol) was carefully added to a mixture of 1-allyl-2,3-isopropylidene-5-chloro-α-D-ribofuranose (0.13 g, 0.57 mmol) and SiEt₃H (108.7 μL, 0.681 mmol) at −78° C. The resulting suspension was stirred at this temperature for 30 minutes, after which it was allowed to warm to rt overnight. The resulting mixture was diluted with water and Et₂O and the aqueous layer was extracted with Et₂O. The combined organic layers were washed with brine and dried over MgSO₄. After removal of the solvent under reduced pressure, the crude product was purified by Prep HPLC using a RP XBridge Prep C18 column with a mobile phase of 0.25% NH₄CO₃ solution in Water: CH₃CN to give the pure compound (4-(5-chloro-α-D-ribofuranose)butyl)boronic acid (30.0 mg, 0.13 mmol, 23%) as a white solid.

C₈H₁₆BClO₅ (238.5 g/mol).

### Peracetyl-β-D-GlcNAc

To an ice-cold stirred suspension of D-GlcNAc (6.42 g, 29.0 mmol) in Ac₂O (80 mL, 74.0 g, 725 mmol) montmorillonite K-10 (24.0 g) was added in portions over 10 mins. The ice-bath was removed, and the reaction mixture stirred at this temperature for 24 h. The reaction mixture was filtered through Celite and the pad washed with AcOEt until colourless. The combined filtrate was concentrated under reduced. The orange residue was recrystallized from MeOH twice to afford the title product as white needles (2.39 g, 6.11 mmol, 131° C., 19.5%).

C₁₆H₂₃NO₁₀ (389.4 g/mol).

### Peracetyl 1-iodoethyl-β-D-GlcNAc

To a solution of peracetyl β-D-GlcNAc (500 mg, 1.28 mmol) and 2-iodoethanol (400 μL, 882 mg, 5.12 mmol) in dry DCM (15 mL) under argon, ytterbium (II) triflate (240 mg, 0.387 mmol) was added. The reaction mixture was heated to reflux overnight. At the point where analysis by TLC (100% EtOAc, Sulfuric Acid development) showed the reaction complete (16 h) by complete consumption of the starting material (R_{f}=0.68), the red reaction mixture was washed with Sat. Aq. NaHCO₃ (3×30 mL) then concentrated. Purification by column chromatography (40% EtOAc in petroleum ether R_{f}=0.33) afforded the title product as a colourless amorphous solid (524 mg, 1.04 mmol, 820%).

C₁₆H₂₄INO₉ (501.3 g/mol).

### 1-Iodoethyl-β-D-GlcNAc

To a solution of peracetyl 1-iodoethyl-β-D-GlcNAc (250 mg, 0.499 mmol) in dry methanol (5 mL), sodium methoxide in methanol (25%, 100 μL) was added and stirred until analysis by TLC (100% EtOAc, sulfuric acid development) had shown the reaction complete by disappearance of the starting material and the appearance of one spot (R_{f} 0.0). Upon completion (30 min) the reaction mixture was neutralised by addition of DOWEX H⁺ and stirred for 5 minutes, the reaction mixture was filtered and then concentrated to 1 mL, which was washed through a silica plug (which had been thoroughly washed with methanol, water/isopropanol/ethyl acetate 1:2:5), volatiles were removed under reduced pressure to afford the title product as a white amorphous solid (153 mg, 0.409 mmol, 82%).

C₁₆H₂₄INO₉ (501.3 g/mol).

### Peracetyl 2-chloro-α-D-GlcNAc

A suspension of N-acetyl-D-glucosamine (25.0 g, 113 mmol) in acetyl chloride (50.0 mL, 55.0 g, 701 mmol) was sealed with a suba seal and balloon then stirred for 17 h at which point analysis by TLC (100% EtOAc, H₂SO₄ development) indicated complete disappearance of starting material (R_{f}0.0) and the appearance of one major product (R_{f} 0.70) and one side product (R_{f} 0.47). The scarlet solution was diluted with DCM (500 mL), washed with saturated aqueous NaHCO₃ (3×500 mL), dried over MgSO₄, then concentrated under reduced pressure (to 50.0 mL), the crude produce was precipitated with sodium dried Et₂O (1.00 L) to afford beige crystals. Purification by flash column chromatography (Pet Ether/EtOAc 40%->65% gradient elution) afforded the title product as a white amorphous solid (17.53 g, 47.9 mmol, 420%).

C₁₄H₂₀ClNO₈ (365.8 g/mol).

### Peracetyl 1-azido-β-D-GlcNAc

To a rapidly stirred solution of Peracetyl 2-chloro-α-D-GlcNAc (500 mg, 1.37 mmol) and tetra(ⁿbutyl) ammonium hydrogen sulfate (464 mg, 1.37 mmol) in EtOAc (5 mL) and saturated aqueous NaHCO₃ (5 mL), sodium azide (267 mg, 4.10 mmol) was added in portions. The reaction mixture was stirred for 1 h at which point analysis by TLC (100% EtOAc, H₂SO₄ Development) indicated complete disappearance of the starting material (R_{f}0.70) and appearance of a single product (R_{f}0.52). The organic fraction was washed with saturated aqueous NaHCO₃ (3×10 mL) and saturated NH₄Cl (10 mL) then volatiles removed under reduced pressure, the white amorphous solid was purified by flash column chromatography (Pet Ether/EtOAc 50%->80% gradient elution) to give the title product as a white amorphous solid (387 mg, 1.04 mmol, 76%).

C₁₄H₂₀N₄O₈ (372.3 g/mol).

### Peracetyl 1-amino-β-D-GlcNAc

To a rapidly stirred solution of peracetyl 1-azido-β-D-GlcNAc (1.00 g, 2.69 mmol) in anhydrous methanol (16 mL) under Ar, NEt₃ (0.9 mL, 0.653 g, 6.45 mmol) and 1,3-propanedithiol (0.6 mL, 0.648 g, 6.00 mmol) were sequentially added. The effervescent reaction mixture was stirred at RT for 2 h, at which point a large amount of white precipitate could be seen suspended in the colourless to pale yellow solution, TLC analysis (10% MeOH/CHCl₃ Anisaldehyde development) showed complete consumption of the starting material (R_{f} 0.72) and the formation of a major spot (R_{f} 0.41). The methanol was removed under reduced pressure and the residue dissolved in chloroform, this was loaded onto a short silica plug and washed with a large amount of chloroform then eluted with MeOH/CHCl₃ (10%), solvents were removed, then the glassy solid stored under high vacuum overnight. The title product was obtained as a colourless glassy solid (0.74 g, 2.15 mmol, 80%).

C₁₄H₂₂N₂O₈ (346.3 g/mol).

### Peracetyl 1-(iodoactamide)-β-D-GlcNAc

To a stirred solution of EEDQ (427 mg, 1.73 mmol) and iodoacetic acid (323 mg, 1.73 mmol) in THF (10 mL) peracetyl 1-amino-β-D-GlcNAc (500 mg, 1.73 mmol) was added. The reaction mixture was stirred at room temperature for 24 h at which point analysis by TLC (5% MeOH/CHCl₃ development with 254 nm and anisaldehyde dip) had shown significant development of product (R_{f}=0.38) The reaction mixture was concentrated to dryness on diatomaceous earth (5.00 g) then loaded onto a chromatography column which had been pre-equilibrated with chloroform then purified by column chromatography (0->10% MeOH/CHCl₃ gradient elution) to afford the title product as a white amorphous solid (410 mg, 951 μmol, 55%), which turned yellow if exposed to light for sustained periods.

C₁₆H₂₃IN₂O₉ (514.3 g/mol).

### 1-(Iodoacetamide)-β-D-GlcNAc

To a solution of peracetyl 1-(iodoacetamide)-β-D-GlcNAc (410 mg, 0.796 mmol) in methanol (8 mL) sodium methoxide in methanol (25%, 200 μL) was added and stirred for 5 minutes at which point analysis by TLC (MeOH/CHCl₃ 30%, anisaldehyde) had shown the reaction complete by disappearance of the starting material (R_{f}0.9) and the appearance of one spot (R_{f}0.45). The reaction mixture was neutralised by addition of DOWEX H⁺ (352 mg) and stirred for 5 minutes, the reaction mixture was filtered andthen concentrated to dryness to afford the title product as a white to pale orange amorphous solid (303 mg, 774 μmol, 98%), which turned brown if exposed to light for sustained periods.

C₁₀H₁₇IN₂O₆ (388.2 g/mol).

### 2-(3-Azidopropyl)-4,4,5,5-tetramethyl-1,3,2-dioxaborolane

A round-bottom flask was charged with 3-bromopropylboronic acid pinacol ester (200 mg, 0.80 mmol), sodium azide (525 mg, 8.00 mmol), tetra-n-butylammonium bromide (130 mg, 0.40 mmol), water (2 mL) and EtOAc (2 mL). The resulting reaction solution was stirred for 16 hours at 85° C. After cooling to room temperature, water (10 mL) was added and the resulting aqueous mixture was extracted with EtOAc (3×10 mL). The combined organic layers were dried over MgSO₄, concentrated under vacuum and purified by CombiFlash Rfflash chromatography system equipped with an 12 g RediSep R_{f} silica gold column (gradient: 2 min 100% hexane then linear gradient to 50% petroleum ether:EtOAc (95:5) over 14 min) to afford the product (137 mg, 0.17 mmol, 81%) as a colorless liquid.

C₉H₁₈BN₃O₂ (211.1 g/mol).

### N-(3-(4,4,5,5-Tetramethyl-1,3,2-dioxaborolan-2-yl)propyl)benzamide

Under nitrogen atmosphere a round-bottom flask was charged with 2-(3-azidopropyl)-4,4,5,5-tetramethyl-1,3,2-dioxaborolane (211 mg, 1.00 mmol) and chloroform (1 mL). Then, 2,6-lutidine (139 mg, 151 μL, 1.30 mmol) and thiobenzoic acid (276 mg, 2.00 mmol) were added to the reaction mixture and stirred for 16 hours at 55° C. Afterwards, the crude reaction mixture was concentrated under vacuum and dissolved in EtOAc (25 mL). The organic layer was washed with sodium bicarbonate solution (sat., 25 mL), water (25 mL), brine (25 mL), dried over MgSO₄ and concentrated under vacuum. The crude product was purified by CombiFlash Rfflash chromatography system equipped with an 12 g RediSep R_{f} silica gold column (gradient: 2 min 100% hexane then linear gradient to 100% petroleum ether:EtOAc (1:3) over 14 min) to afford the product (50 mg, 0.17 mmol, 17%) as a white solid.

C₁₆H₂₄BNO₃ (289.2 g/mol).

### N-(3-(4,4,5,5-Tetramethyl-1,3,2-dioxaborolan-2-yl)propyl)acetamide

Under nitrogen atmosphere a round-bottom flask was charged with 2-(3-azidopropyl)-4,4,5,5-tetramethyl-1,3,2-dioxaborolane (211 mg, 1.00 mmol) and chloroform (1 mL). Then, 2,6-lutidine (139 mg, 151 μL, 1.30 mmol) and thioacetic acid (152 mg, 142 μL, 2.00 mmol) were added to the reaction mixture and stirred for 16 hours at 55° C. Afterwards, the crude reaction mixture was concentrated under vacuum and dissolved in EtOAc (25 mL). The organic layer was washed with sodium bicarbonate solution (sat., 25 mL), water (25 mL), brine (25 mL), dried over MgSO₄ and concentrated under vacuum. The crude product was purified by CombiFlash Rfflash chromatography system equipped with an 12 g RediSep R_{f} silica gold column (gradient: 2 min 100% hexane then linear gradient to 100% EtOAc over 14 min and 6 min 100% EtOAc) to afford the product (60 mg, 0.26 mmol, 26%) as a black oil.

C₁₁H₂₂BNO₃ (227.1 g/mol).

### 2-(3-Iodopropyl)-4,4,5,5-tetramethyl-1,3,2-dioxaborolane

A round-bottom flask was charged with 3-bromopropylboronic acid pinacol ester (500 mg, 2.00 mmol), sodium iodide (900 mg, 6.00 mmol and acetone (5 mL). The resulting reaction solution was stirred for 16 hours at 60° C. After cooling to room temperature, water (25 mL) was added and the resulting aqueous mixture was extracted with EtOAc (3×25 mL). The combined organic layers were washed with an aqueous solution of sodium hydrosulfite (sat., 2×25 mL), water (25 mL), brine (25 mL), dried over MgSO₄, concentrated under vacuum and purified by CombiFlash R_{f} flash chromatography system equipped with an 12 g RediSep R_{f} silica gold column (gradient: 2 min 100% hexane then linear gradient to 100% petroleum ether:EtOAc (97:3) over 14 min) to afford the product (430 mg, 1.45 mmol, 73%) as a colorless liquid.

C₉H₁₈BIO₂ (296.0 g/mol).

### 3-Aminopropylboronic acid pinacol ester

3-Azidopropylboronic acid pinacol ester (1.00 g, 4.70 mmol) was added to EtOH (15 mL) followed by 10% Pd on activated C (80 mg). Argon was bubbled through for 15 min before the reaction mixture was purged with hydrogen for a further 15 min and left to stir at RT for 24 h under a hydrogen balloon. The mixture was filtered through celite and the solvent was removed under reduced pressure. The residue was washed with cold ether and filter, leaving a white powder (310 mg, 1.68 mmol, 36%).

C₉H₂₀BNO₂ (185.1 g/mol).

### 3-Trimethylaminopropylboronic acid pinacol ester iodide

3-Aminopropylboronic acid pinacol ester (150 mg, 810 μmol) was dissolved in MeOH (8 mL). 2 M LiGH (2.43 mL, 4.86 mmol) followed by Mel (0.5 mL, 8.10 mmol) was added dropwise and stirred at RT for 1.5 h. Solvents were removed under reduced pressure and the resultant white solid was extracted with acetonitrile, taking the desired product into solution. Evaporation under reduced pressure followed by trituration with DCM where the filtrate was then evaporated and extracted with acetone gave a pale yellow oil (105 mg, 0.38 mmol, 47%).

C₆H₁₈BINO₂ (273.9 g/mol).

### 2-Acetylamino-N-benzyl-acrylamide

To a stirred solution of 2-acetamidoacrylic acid (1.29 g, 10.0 mmol, 1.00 equiv.) and 4-methylmorpholine (1.21 mL, 11.0 mmol, 1.10 equiv.) in THE (100 mL) were added subsequently isobutyl chloroformate (1.43 mL, 11 mmol, 1.10 equiv.) and benzylamine (1.20 mL, 11.0 mmol, 1.10 equiv.). The mixture was stirred at room temperature for 2 h, before it was filtered and the solvent was evaporated. The residue was purified by flash chromatography (n-heptane/EtOAc; 10-100% EtOAc) yielding the title compound as a white solid (1.62 g, 7.43 mmol, 74%).

C₁₂H₁₄N₂O₂ (218.3 g/mol).

### (2-Acetamido-3-(benzylamino)-3-oxopropyl)boronic acid

To a stirred solution of 2-acetylamino-N-benzyl-acrylamide (100 mg, 0.46 mmol) in dry THE (5 mL) was added BH₃·THF (1 M, 0.9 mL, 0.92 mmol) at 0° C. The mixture was stirred at 0° C. for 10 min before being allowed to warm to room temperature. The reaction mixture was stirred for 3 days and quenched by addition of 500 μL of water. The solvent was evaporated. The residual liquid lyophilized and dissolved in H₂O for purification. Purification was performed via preparative HPLC (Stationary phase: RP XBridge Prep C18 OBD-10 μm, 50×250 mm, Mobile phase: 0.25% NH₄HCO₃ solution in water, MeCN). The title compound was afforded after lyophilisation as a white solid (16.6 mg, 0.06 mmol, 14%).

C₁₂H₁₇BN₂O₄ (264.0 g/mol).

BPin-Biotin

NEt₃ (28 μL) was added to a solution of 3-aminopropylboronic acid pinacol ester (15 mg, 81 μmol) and the active biotin ester (44 mg, 69 μmol) in anhydrous DCM under argon. The reaction mixture was left to stir overnight at rt and then concentrated. Purification by flash column chromatography (CHCl₃/MeOH 0->10% gradient elution) gave the title product as a white solid (12 mg, 26%).

C₃₀H₅₅BN₄O₉S (658.7 g/mol).

### 3-(4,4,5,5-Tetramethyl-1,3,2-dioxaborolan-2-yl)propanoic acid

To a solution of 2-tBu-ethylboronic acid pinacol ester (500 mg, 1.95 mmol) in CH₂Cl₂ (1.5 mL) was added trifluoroacetic acid (1.5 mL). The solution was stirred at room temperature for 2 h before being concentrated under a stream of nitrogen and azeotroped from more CH₂Cl₂ to yield the desired carboxylic acid as a viscous oil in quantitative yield which was used without further purification.

C₉H₁₇BO₄ (200.0 g/mol).

### 2-((1,1-Difluoroethyl)sulfonyl)pyridine

Under nitrogen atmosphere a heat-gun dried two-neck flask was charged with difluoromethyl 2-pyridyl sulfone (193 mg, 1.00 mmol), THE (4 mL) and DMI(0.4 mL). Then, the reaction mixture was cooled to −78° C. in an iso-propanol/dry ice mixture followed by addition of methyl iodide (766 mg, 0.33 μL, 5.40 mmol) and dropwise addition of LiHMDS (1M in THF, 2.5 mL, 2.50 mmol) and after complete addition the mixture was stirred for 30 minutes at −78° C. After quenching with aqueous ammonium chloride solution (sat., 5 mL) the resulting aqueous solution was extracted with ethyl acetate (3×10 mL). The combined organic layers was dried over MgSO₄, concentrated under vacuum and the crude product was purified by column chromatography (SiO₂, hexane:ethyl acetate (3:1), d×h: 3.5×13 cm) to afford the product (123 mg, 0.59 mmol, 59%) as a yellow solid.

C₇H₇F₂N₂O₂S (207.2 g/mol).

### tert-Butyl (3,3-difluoro-3-(pyridin-2-ylsulfonyl)propyl)carbamate

Under nitrogen atmosphere a heat-gun dried two-neck flask was charged with difluoromethyl 2-pyridyl sulfone (1.04 g, 5.38 mmol), 3-Boc-1,2,3-oxathiazolidine 2,2-dioxide (1 g, 4.48 mmol), THE (20 mL) and DMI (2 mL). Then, the reaction mixture was cooled to −95° C. in methanol/liquid nitrogen mixture followed by dropwise addition of LiHMDS (1M in THF, 5.5 mL, 5.5 mmol) and after complete addition the mixture was stirred at −95° C. After 30 minutes, the reaction mixture was quenched by addition of sulfuric acid (1M, 20 mL), allowed to warm to room temperature and stirred for three hours. At 0° C., the reaction mixture was adjusted to an alkaline pH (>10) by addition of aqueous NaOH solution (1M) and the resulting aqueous mixture was extracted with EtOAc (3×100 mL). The combined organic layers were washed with aqueous LiCl solution (sat., 20 mL), brine (20 mL), dried over MgSO₄ and concentrated under vacuum. The product was purified by a comi to afford the product (630 mg, 1.88 mmol, 42%) as a yellow solid.

C₁₃H₁₈F₂N₂O₄S (336.4 g/mol).

### Benzyl (3,3-difluoro-3-(pyridin-2-ylsulfonyl)propyl)carbamate

Under nitrogen atmosphere a heat-gun dried two-neck flask was charged with difluoromethyl 2-pyridyl sulfone (350 mg, 1.82 mmol), benzyl 1,2,3-oxathiazolidine-3-carboxylate 2,2-dioxide (700 mg, 2.75 mmol), THF (7 mL) and DMI (0.7 mL). Then, the reaction mixture was cooled to −78° C. in an iso-propanol/dry ice mixture followed by dropwise addition of LiHMDS (1M in THF, 2.2 mL, 2.2 mmol) and after complete addition the mixture was stirred at −78° C. After 30 minutes, the reaction mixture was quenched by addition of sulfuric acid (1M, 10 mL), allowed to warm to room temperature and stirred for three hours. At 0° C., the reaction mixture was adjusted to an alkaline pH (>10) by addition of NaOH solution (1M) and the resulting aqueous mixture was extracted with EtOAc (3×50 mL). The combined organic layers were washed with aqueous LiCl solution (sat., 10 mL), brine (10 mL), dried over MgSO₄ and concentrated under vacuum. The crude product was purified by using a CombiFlash Rfflash chromatography system equipped with an 4 g RediSep R_{f} silica gold column (gradient: 2 min 100% petrol ether then linear gradient to 100% EtOAc over 14 min) to afford the product (290 mg, 0.79 mmol, 43%) as a white solid.

C₁₆H₁₆F₂N₂O₄S (370.4 g/mol).

### 3,3-Difluoro-3-(pyridin-2-ylsulfonyl)propan-1-aminium trifluoroacetate

Under nitrogen atmosphere a round-bottom flask was charged with tert-butyl (3,3-difluoro-3-(pyridin-2-ylsulfonyl)propyl)carbamate (230 mg, 0.69 mmol) and DCM (5 mL). Then, the reaction mixture was cooled to 0° C. in an ice-water bath followed by dropwise addition of TFA (1.19 g, 800 μL, 10.5 mmol) and after complete addition the mixture was stirred for two hours at 0° C. Afterwards, the crude reaction mixture was concentration under vacuum and dried on the high vacuum to afford the product (231 mg, 0.69 mmol, 100%) as a yellow solid.

C₁₀H₁₁F₅N₂O₄S (350.4 g/mol).

### N-(3,3-Difluoro-3-(pyridin-2-ylsulfonyl)propyl)acetamide

Under nitrogen atmosphere a heat-gun dried two-neck flask was charged with 3,3-difluoro-3-(pyridin-2-ylsulfonyl)propan-1-aminium trifluoroacetate (202 mg, 0.60 mmol), DCM (7 mL) and. DIPEA (263 mg, 355 μL, 2.04 mmol) followed by dropwise addition of acetic anhydride (76.7 mg, 71 μL, 0.75 mmol). After stirring for two hours at room temperature the reaction mixture was concentration under vacuum. Then, the crude mixture was dissolved in DCM (25 mL) and the resulting organic layer was washed with NaOH (2M, 20 mL), HCl (1M, 20 mL), brine (20 mL), dried over MgSO₄ and concentrated under vacuum. The crude product was purified by using a CombiFlash R_{f} flash chromatography system equipped with an 4 g RediSep R_{f} silica gold column (gradient: 2 min 100% petrol ether then linear gradient to 100% EtOAc over 14 min and 5 min 100% EtOAc) to afford the product (110 mg, 0.40 mmol, 66%) as a pale yellow solid.

C₁₀H₁₂F₂N₂O₃S (278.3 g/mol).

### tert-Butyl (3,3-difluoro-3-(pyridin-2-ylsulfonyl)propyl)(methyl)carbamate

Under nitrogen atmosphere a heat-gun dried two-neck flask was charged with tert-butyl (3,3-difluoro-3-(pyridin-2-ylsulfonyl)propyl)carbamate (241 mg, 0.72 mmol) and DMF (8 mL). Then, the reaction mixture was cooled to 0° C. in an ice/water bath followed by addition of Mel (204 mg, 90.0 μL, 1.44 mmol) and NaH (60% in mineral oil, 43 mg, 1.08 mmol) and the resulting mixture was stirred for six hours at room temperature. The crude reaction mixture was quenched by addition of water (25 mL) and the resulting aqueous mixture was extracted with EtOAc (3×25 mL). The combined organic layers were washed with water (25 mL), brine (25 mL), dried over MgSO₄ and concentrated under vacuum. The crude product was purified by using a CombiFlash Rfflash chromatography system equipped with an 4 g RediSep R_{f} silica gold column (gradient: 2 min 100% petrol ether then linear gradient to 100% EtOAc/petroleum ether (4:5) over 14 min) to afford the product (210 mg, 0.60 mmol, 83%) as a yellow gum.

C₁₄H₂₀F₂N₂O₄S (350.4 g/mol).

### 3,3-Difluoro-N-methyl-3-(pyridin-2-ylsulfonyl)propan-1-aminium trifluoroacetate

A round-bottom flask was charged with tert-butyl (3,3-difluoro-3-(pyridin-2-ylsulfonyl)-propyl)(methyl)carbamate (175 mg, 0.50 mmol) and CH₂Cl₂ (5 mL). Then, the reaction mixture was cooled in an ice-water bath followed by dropwise addition of TFA (1.19 g, 0.80 mL, 10.5 mmol) and stirred over night at room temperature. Afterwards the crude mixture was concentrated and dried under vacuum to afford the product (182 mg, 0.50 mmol, 100%) as a yellow oil.

C₁₁H₁₃F₅N₂O₄S (364.3 g/mol):

### 3,3-Difluoro-N,N-dimethyl-3-(pyridin-2-ylsulfonyl)propan-1-amine

A round-bottom flask was charged with 3,3-difluoro-3-(pyridin-2-ylsulfonyl)propan-1-aminium trifluoroacetate (70 mg, 0.20 mmol) and MeOH (2 mL). Then, formaldehyde (37 wt. % in H₂O, 66.6 mg, 180 μL, 2.22 mmol) was added and the resulting reaction mixture was stirred for 10 minutes at room temperature followed by addition of sodium triacetoxyborohydride (179 mg, 0.84 mmol). After stirring for further 16 hours at room temperature the reaction mixture was concentrated under vacuum. The crude product was purified by using a CombiFlash Rfflash chromatography system equipped with an 4 g RediSep R_{f} silica gold column (gradient: 2 min 100% CH₂Cl₂ then linear gradient to 100% CH₂Cl₂/MeOH (1:1) over 14 min) to afford the product (40 mg, 0.15 mmol, 76%) as a pale yellow liquid.

C₁₀H₁₄F₂N₂O₂S (264.3 g/mol).

### 3,3-Difluoro-N,N,N-trimethyl-3-(pyridin-2-ylsulfonyl)propan-1-aminium

round-bottom flask was charged with 3,3-difluoro-3-(pyridin-2-ylsulfonyl)propan-1-aminium trifluoroacetate (150 mg, 0.43 mmol), MeCN (2.7 mL) and MeOH (1.3 mL). Then, DIPEA (332 mg, 448 μL, 2.57 mmol) and Mel (609 mg, 267 μL, 4.29 mmol) were added and the resulting reaction mixture was stirred for 30 hours at room temperature. Afterwards the crude mixture was concentrated and dried under vacuum. The resulting crude solid was triturated with a solution of chloroform and MeOH (10%) and the white solid was filtered off. The white solid was washed with a solution of chloroform and MeOH (10%) and dried under vacuum to afford the product (110 mg, 0.39 mmol, 92%) as a white solid.

C₁₁H₁₇F₂N₂O₂S (279.1 g/mol).

### 2-((Difluoro(methylthio)methyl)sulfonyl)pyridine

Under nitrogen atmosphere a heat-gun dried two-neck flask was charged with difluoromethyl 2-pyridyl sulfone (500 mg, 2.59 mmol), THE (10 mL), DMI (1 mL) and S-methyl methanethiosulfonate (488 mg, 368 μL, 3.90 mmol). Then, the reaction mixture was cooled to −78° C. in an iso-propanol/dry ice mixture followed by dropwise addition of LiHMDS (1M in THF, 3.2 mL, 3.20 mmol) and after complete addition the mixture was stirred for 30 minutes at −78° C. After quenching with aqueous ammonium chloride solution (sat., 10 mL) the resulting aqueous solution was extracted with ethyl acetate (3×25 mL). The combined organic layers were washed with aqueous LiCl solution (sat. 25 mL), brine (25 mL), dried over MgSO₄ and concentrated under vacuum. The crude product was purified by using a CombiFlash Rfflash chromatography system equipped with an 12 g RediSep R_{f} silica gold column (gradient: 2 min 100% CHCl₃/heptane (1:1) then linear gradient to 100% CHCl₃/heptane/EtOAc (3:3:1) over 14 min) to afford the product (500 mg, 2.10 mmol, 81%) as a white solid.

C₇H₇F₂NO₂S₂ (239.3 g/mol).

### 2-((Difluoro(methylsulfinyl)methyl)sulfonyl)pyridine

Under nitrogen atmosphere a heat-gun dried round-bottomed neck flask was charged with 2-((difluoro(methylthio)methyl)sulfonyl)pyridine (180 mg, 0.75 mmol) and CH₂Cl₂ (3 mL). Then, the reaction mixture was cooled to 0° C. in an ice/water mixture followed by dropwise addition of 3-chloroperbenzoic acid (<77%, 186 mg, 0.82 mmol) in CH₂Cl₂ (1 mL) and after complete addition the mixture was stirred for 16 hours at room temperature. The crude mixture was concentrated under vacuum, dissolved in EtOAc (30 mL) and the organic layer was were washed with aqueous NaHCO₃ solution (sat., 2×30 mL), water (30 mL), brine (30 mL), dried over MgSO₄ and concentrated under vacuum. The crude product was purified by using a CombiFlash Rfflash chromatography system equipped with an 12 g RediSep R_{f} silica gold column (gradient: 2 min 100% hexane then linear gradient to 100% petroleum ether/EtOAc (4:5) over 14 min) to afford the product (110 mg, 0.43 mmol, 56%) as a colorless liquid.

C₇H₇F₂NO₃S₂ (255.3 g/mol).

### 2-((Difluoro(methylsulfonyl)methyl)sulfonyl)pyridine

Under nitrogen atmosphere a round-bottom flask was charged with 2-((difluoro(methylthio)methyl)sulfonyl)pyridine (100 mg, 0.42 mmol), MeCN (2 mL), CH₂Cl₂ (1 mL) and water (3 mL). Then, the reaction mixture was cooled to 0° C. in an ice-water mixture followed by addition of sodium periodate (411 mg, 1.93 mmol) and RuCl₃×H₂O (1 mg) and the mixture was stirred for 16 hours. After dilution with water (30 mL) the resulting aqueous solution was extracted with ethyl acetate (3×30 mL). The combined organic layers were washed with water (25 mL), brine (25 mL), dried over MgSO₄ and concentrated under vacuum. The crude product was purified by using a CombiFlash Rfflash chromatography system equipped with an 4 g RediSep R_{f} silica gold column (gradient: 2 min 100% petrol ether then linear gradient to 100% petrol ether/EtOAc (4:3) over 12 min) to afford the product (108 mg, 0.40 mmol, 95%) as a white solid.

C₇H₇F₂NO₄S₂ (271.3 g/mol).

### 3,3-Difluoro-3-(pyridin-2-ylsulfonyl)propan-1-ol) and 3,3-difluoro-3-(pyridin-2-ylsulfonyl)-propyl acetate

Under nitrogen atmosphere a heat-gun dried two-neck flask was charged with difluoromethyl 2-pyridyl sulfone (965 mg, 5.00 mmol), 1,3,2-dioxathiolane 2,2-dioxide (931 mg, 7.50 mmol), THE (20 mL) and DMI (2 mL). Then, the reaction mixture was cooled to −78° C. in an iso-propanol/dry ice mixture followed by dropwise addition of LiHMDS (1M in THF, 6.00 mL, 6.00 mmol) and after complete addition the mixture was stirred at −78° C. After 30 minutes, the reaction mixture was quenched by addition aqueous ammonium acetate (1M, 10 mL), allowed to warm to room temperature and stirred for three hours. At 0° C., the reaction mixture was adjusted to an alkaline pH (>10) by addition of NaOH solution (1M) and the resulting aqueous mixture was extracted with EtOAc (3×50 mL). The combined organic layers were washed with aqueous LiCl solution (sat., 10 mL), brine (10 mL), dried over MgSO₄ and concentrated under vacuum. The crude product was purified by using a CombiFlash Rfflash chromatography system equipped with an 24 g RediSep R_{f} silica gold column (gradient: 2 min 100% petrol ether then linear gradient to 100% EtOAc over 14 min) to afford 3,3-difluoro-3-(pyridin-2-ylsulfonyl)propan-1-ol) (210 mg, 0.89 mmol, 18%) as a white solid and 3,3-difluoro-3-(pyridin-2-ylsulfonyl)-propyl acetate (400 mg, 1.43 mmol, 29%) as a colorless liquid. Analytical data for 3,3-difluoro-3-(pyridin-2-ylsulfonyl)propan-1-ol):

C₈H₉F₂NO₃S (237.2 g/mol).

Analytical data for 3,3-difluoro-3-(pyridin-2-ylsulfonyl)-propyl acetate:

C₁₀H₁₁F₂NO₄S (279.3 g/mol).

### 3,3-Difluoro-3-(pyridin-2-ylsulfonyl)propyl hydrogen sulfate

Under nitrogen atmosphere a heat-gun dried two-neck flask was charged with difluoromethyl 2-pyridyl sulfone (1.93 g, 10.0 mmol), 1,3,2-dioxathiolane 2,2-dioxide (1.86 g, 15.0 mmol), THF (40 mL) and DMI (4 mL). Then, the reaction mixture was cooled to −78° C. in an iso-propanol/dry ice mixture followed by dropwise addition of LiHMDS (1M in THF, 12.0 mL, 12.0 mmol) and after complete addition the mixture was stirred at −78° C. After 30 minutes, the reaction mixture was quenched by addition formic acid in water (1%, 10 mL), allowed to warm to room temperature and concentrated under vacuum. The crude product was purified by using a CombiFlash Rfflash chromatography system equipped with an 80 g RediSep R_{f} silica gold column (gradient: 2 min 100% CHCl₃ then linear gradient to 100% CHCl₃/MeOH (1:1) over 14 min) to afford the product (3.00 g, 9.46 mmol, 95%) as a yellow solid.

C₈H₉F₂NO₆S₂ (317.3 g/mol).

### 3,3-Difluoro-3-(pyridin-2-ylsulfonyl)propyl 4-methylbenzenesulfonate

A round-bottom flask was charged with 3,3-difluoro-3-(pyridin-2-ylsulfonyl)propyl hydrogen sulfate (1.00 g, 3.16 mmol) and THE (24 mL). After addition of hydrochloric acid (37%, 1.60 mL) the reaction mixture was stirred at room temperature for 16 hours. Then, the crude mixture was cooled in an ice-water bath and quenched with aqueous NaHCO₃ solution (sat., 30 mL). The resulting aqueous solution was extracted with EtOAc (3×25 mL), the combined organic layers were washed with brine (25 mL), dried over MgSO₄ and concentrated under vacuum to afford the crude alcohol (745 mg, 3.14 mmol, 100%) as yellow solid.

The crude alcohol was dissolved in CH₂Cl₂ (25 mL) and cooled in an ice-water batch. At 0° C., triethylamine (850 mg, 617 μL, 6.10 mmol) and 4-toluenesolfonyl chloride (700 mg, 3.67 mmol) were added and the resulting reaction solution was stirred over night in the ice-water bath. Then, the mixture was quenched by addition of aqueous hydrochloric acid solution (1M, 30 mL) and the aqueous layer was extracted with CH₂Cl₂ (3×25 mL), the combined organic layers were washed with water (30 mL), brine (30 mL), dried over MgSO₄ and concentrated under vacuum. The crude product was purified by using a CombiFlash Rfflash chromatography system equipped with an 24 g RediSep R_{f} silica gold column (gradient: 2 min 100% petrol ether then linear gradient to 100% EtOAc/petrol ether (3:2) over 14 min) to afford the product (825 mg, 2.09 mmol, 66%) as a white solid.

C₁₅H₁₅F₂NO₅S₂ (391.4 g/mol).

### 2-((3-Azido-1,1-difluoropropyl)sulfonyl)pyridine

A round-bottom flask was charged with 3,3-difluoro-3-(pyridin-2-ylsulfonyl)propyl 4-methylbenzenesulfonate (370 mg, 0.95 mmol), DMF (10 mL) and sodium azide (308 mg, 4.75 mmol). After stirring for three hours at 85° C. the reaction mixture was diluted with water (30 mL). The aqueous mixture was extracted with EtOAc (3×25 mL), the combined organic layers were washed with water (3×25 mL), brine (2×25 mL), dried over MgSO₄ and concentrated under vacuum to afford the product (203 mg, 0.77 mmol, 82%) as yellow liquid.

C₈H₈F₂N₄O₂S (262.2 g/mol).

### 2-((1,1-Difluoro-3-iodopropyl)sulfonyl)pyridine

round-bottom flask was charged with 3,3-difluoro-3-(pyridin-2-ylsulfonyl)propyl 4-methylbenzenesulfonate (391 mg, 1.00 mmol), acetone (10 mL) and sodium iodide (749 mg, 5.00 mmol). After stirring for six hours at 60° C. the reaction mixture was concentrated and then diluted with water (30 mL). The aqueous mixture was extracted with EtOAc (3×25 mL), the combined organic layers were washed with an aqueous solution of Na₂S₂O₃ (sat., 2×25 mL), water (25 mL), brine (25 mL), dried over MgSO₄ and concentrated under vacuum to afford the product (277 mg, 0.88 mmol, 88%) as yellow liquid.

C₈H₈F₂INO₂S (347.1 g/mol).

### 2-((Difluoroiodomethyl)sulfonyl)pyridine

Under nitrogen atmosphere a heat-gun dried two-neck flask was charged with difluoromethyl 2-pyridyl sulfone (290 mg, 1.50 mmol), THE (6 mL) and DMI(0.6 mL). Then, the reaction mixture was cooled to −78° C. in an iso-propanol/dry ice mixture followed by addition of diiodoethane (1.06 g, 3.75 mmol) and dropwise addition of LiHMDS (1M in THF, 3.75 mL, 3.75 mmol) and after complete addition the mixture was stirred for 30 minutes at −78° C. After quenching with aqueous ammonium chloride solution (sat., 10 mL) the resulting aqueous solution was extracted with chloroform (3×25 mL). The combined organic layers was dried over MgSO₄, concentrated under vacuum and the crude product was purified by CombiFlash R_{f} flash chromatography system equipped with an 12 g RediSep R_{f} silica gold column (gradient: 2 min 100% petrol ether then linear gradient to 100% EtOAc over 14 min to afford the product (223 mg, 0.70 mmol, 47%) as a yellow solid.

C₆H₄F₂INO₂S (319.1 g/mol).

### 2-Bromo-2,2-difluoroacetamide

A round-bottom flask was charged with ethyl bromodifluoroacetate (1.58 g, 1.0 mL, 7.78 mmol) and methanol (5 mL). Then, the reaction mixture was cooled to −15° C. in an sodium chloride/ice mixture followed by dropwise addition of ammonia in methanol (7N, 2.5 mL). After stirring for 48 hours at room temperature, the crude mixture was concentrated and dried under vacuum to afford the product (1.25 g, 92%) as a white solid.

C₂H₂BrF₂NO (173.9 g/mol).

### Sodium 2-bromo-2,2-difluoroacetate

A round-bottom flask was charged with sodium hydroxide (300 mg, 7.71 mmol) and methanol (7 mL). Then, the reaction mixture was cooled to 0° C. in an ice-water bath followed by dropwise addition of ethyl bromodifluoroacetate (1.58 g, 1.0 mL, 7.78 mmol). After stirring for 16 hours at room temperature, the crude mixture was concentrated and dried under vacuum to afford the product (1.30 g, 86%) as a white solid.

C₂BrF₂Na (196.9 g/mol).

### Ethyl 2-fluoro-2-(pyridin-2-ylsulfonyl)acetate

Under nitrogen atmosphere a heat-gun round-bottom flask was charged with 2-mercaptopyridine (1.50 g, 13.5 mmol) and ethanol (34 mL). Then, the reaction mixture was cooled to 0° C. in an ice/water bath followed by dropwise addition of triethylamine (1.38 g, 1.90 mL, 13.5 mmol). After stirring for 10 minutes, ethyl bromofluoroacetate (2.50 g, 1.6 mL, 13.49 mmol) was added dropwise and the resulting mixture was stirred for 16 hours at room temperature. Afterwards the crude mixture was quenched by addition of aqueous hydrochloric acid solution (1M, 50 mL) and the aqueous layer was extracted with dichloromethane (3×50 mL). The combined organic layers were washed with brine (50 mL), dried over MgSO₄, concentrated under vacuum and the crude product was purified by column chromatography (SiO₂, petrol ether:ethyl acetate (6:1), d×h: 6×9.5 cm) to afford the sulfide precursor (2.81 g, 13.1 mmol, 97%) as colorless oil.

A round-bottom flask was charged with sulfide precursor (1.00 g, 4.65 mmol), acetonitrile (6 mL), dichloromethane (6 mL) and water (15 mL). Then, sodium periodate (4.50 g, 21.4 mmol) and ruthenium chloride hydrate (3 mg) were added to the reaction mixture and the resulting solution was stirred for 16 hours at room temperature. Afterwards the crude mixture was diluted with water (50 mL) and the aqueous mixture was extracted with ether (3×50 mL). The combined organic layers were washed with brine (50 mL), dried over MgSO₄, concentrated under vacuum and the crude product was purified by column chromatography (SiO₂, dichloromethane:chloroform (20:1), d×h: 6×12 cm) to afford the product (1.05 g, 4.25 mmol, 91%) as colorless oil.

C₉H₁₀FNO₄S (247.2 g/mol).

### 2-Fluoro-2-(pyridin-2-ylsulfonyl)acetamide

A round-bottom flask was charged with ethyl 2-fluoro-2-(pyridin-2-ylsulfonyl)acetate (490 mg, 1.98 mmol) and ethanol (6 mL). Then, the reaction mixture was cooled to 0° C. in an ice-water bath followed by dropwise addition of ammonia in methanol (7N, 4.00 mL). After stirring for 30 minutes at room temperature, the crude mixture was concentrated under vacuum. Afterwards the resulting solid was triturated with ethyl acetate/hexane (4:2, 6 mL) and to afford the product (380 mg, 84%) as a white solid after drying on vacuum.

C₇H₇FN₂O₃S (218.2 g/mol).

### Sodium 2-fluoro-2-(pyridin-2-ylsulfonyl)acetate

A round-bottom flask was charged with ethyl 2-fluoro-2-(pyridin-2-ylsulfonyl)acetate (450 mg, 1.82 mmol), MeOH (8 mL) and THE (8 mL). Then, aqueous sodium hydroxide solution (1M, 1.9 mL) was added dropwise to the reaction mixture and stirred for 10 minutes. The crude mixture was concentrated and dried under vacuum to afford the product (424 mg, 97%) as a white solid.

C₇H₅FNNaO₄S (241.2 g/mol).

### 2-(Ethylsulfonyl)pyridine

Under nitrogen atmosphere a heat-gun round-bottom flask was charged with 2-mercaptopyridine (3.1 g, 27.8 mmol), THE (56 mL) and MeCN (56 mL). Then, the reaction mixture was cooled to 0° C. in an ice/water bath followed by dropwise addition of DBU (4.68 g, 4.60 mL, 30.8 mmol). After stirring for five minutes, ethyl iodide (6.50 g, 3.35 mL, 41.7 mmol) was added dropwise and the resulting mixture was stirred for 16 hours at room temperature. Afterwards the crude mixture was diluted with water (200 mL), extracted with EtOAc (3×50 mL), the combined organic layers were washed with water (50 mL), aqueous HCl (1M, 50 mL), brine (50 mL), dried over MgSO₄, concentrated under vacuum to afford the crude sulfide (750 mg) as yellow oil.

A round-bottom flask was charged with crude sulfide (750 mg), acetonitrile (30 mL), dichloromethane (10 mL), water (40 mL) and cooled 0° C. in an ice/water bath. Then, sodium periodate (5.30 g, 24.9 mmol) and ruthenium chloride hydrate (5 mg) were added to the reaction mixture and the resulting solution was stirred for 16 hours at room temperature. Afterwards the crude mixture was diluted with water (40 mL) and the aqueous mixture was extracted with EtOAc (3×60 mL). The combined organic layers were washed with water (50 mL), brine (50 mL), dried over MgSO₄, concentrated under vacuum and the crude product was purified by CombiFlash Rfflash chromatography system equipped with an 24 g RediSep R_{f} silica gold column (gradient: 2 min 100% hexane then linear gradient to 100% petroleum ether/EtOAc (1:1) over 14 min to afford the product (430 mg, 2.51 mmol, 9%) as a yellow oil.

C₇H₉NO₂S (171.2 g/mol).

### 2-((1-Fluoroethyl)sulfonyl)pyridine

Under nitrogen atmosphere a heat-gun dried two-neck flask was charged with 2-(ethylsulfonyl)pyridine (350 mg, 1.82 mmol), benzyl 1,2,3-oxathiazolidine-3-carboxylate 2,2-dioxide (400 mg, 2.33 mmol) and THE (10 mL). Then, the reaction mixture was cooled to −78° C. in an iso-propanol/dry ice mixture followed by addition of NFSI (880 mg, 2.80 mmol) and dropwise addition of LiHMDS (1M in THF, 2.5 mL, 2.5 mmol) and after complete addition the mixture was stirred at −78° C. for 90 minutes and further 90 minutes at room temperature. Afterwards, the reaction mixture was quenched by addition of aqueous NH₄Cl solution (sat., 20 mL) and extracted with EtOAc (3×25 mL). The combined organic layers were washed with aqueous NaHCO₃ solution (sat., 30 mL), water (30 mL), brine (30 mL), dried over MgSO₄ and concentrated under vacuum. The crude product was purified by using a CombiFlash Rfflash chromatography system equipped with an 4 g RediSep R_{f} silica gold column (gradient: 2 min 100% petroleum ether then linear gradient to 100% petroleum ether:EtOAc (5:4) over 14 min) to afford the product (138 mg, 0.73 mmol, 31%) as a colorless liquid.

C₇H₈FNO₂S (189.2 g/mol).

### Ethyl 2,2-difluoro-2-(pyridin-2-ylthio)acetate

A round-bottom flask was charged with cesium carbonate (23.5 g, 72.0 mmol) and heated with a heat-gun three times for 10 minutes under vacuum. Then, under nitrogen atmosphere DMF (340 ml), 2-mercaptopyridine (4.00 g, 36.0 mmol) and ethyl bromodifluoroacetate (14.6 g, 9.23 mL, 72.0 mmol) were added and the resulting mixture was stirred for 18 hours at room temperature. Afterwards the reaction mixture was diluted with water (300 mL) and the aqueous mixture was extracted with EtOAc (3×200 mL). The combined organic layers were washed with water (100 mL) brine (100 mL), dried over MgSO₄ and concentrated under vacuum. The crude product was purified by CombiFlash Rfflash chromatography system equipped with an 80 g RediSep R_{f} silica gold column (gradient: 2 min 100% petrol ether then linear gradient to 100% petrol ether/EtOAc (5:1) over 14 min) to afford the product (6.30 g, 27.0 mmol, 75%) as a yellow liquid.

C₉H₉F₂NO₂S (233.2 g/mol).

### 2,2-Difluoro-2-(pyridin-2-ylthio)ethan-1-ol

Under nitrogen atmosphere a heat-gun dried round-bottom flask was charged with ethyl 2,2-difluoro-2-(pyridin-2-ylthio)acetate (3.00 g, 12.9 mmol), THF (7.5 mL) and EtOH (52.5 mL). Then, the reaction mixture was cooled to 0° C. in an ice/water bath followed by addition of sodium borohydride (583 mg, 15.4 mmol) and the resulting mixture was stirred for one hour at 0° C. Afterwards the crude mixture was quenched by addition of aqueous hydrochloric acid solution (1M, 15 mL) and the solvent was removed under vacuum. The aqueous layer was extracted with EtOAc (3×60 mL) and the combined organic layers were washed with brine (50 mL) dried over MgSO₄, concentrated under vacuum to give the product (2.25 g, 11.8 mmol, 91%) as a yellow liquid.

C₇H₇F₂NOS (191.2 g/mol).

### 2,2-Difluoro-2-(pyridin-2-ylsulfonyl)ethan-1-ol

Under nitrogen atmosphere a heat-gun dried round-bottom flask was charged with 2,2-difluoro-2-(pyridin-2-ylthio)ethan-1-ol (2.25 g, 10.9 mmol) and CH₂Cl₂ (100 mL). Then, the reaction mixture was cooled to 0° C. in an ice/water bath followed by portion wise addition of meta-chloroperoxybenzoic acid (4×1.55 g, 27.2 mmol) and stirred for 16 hours in the cooling bath. Afterwards the crude mixture was quenched by addition of aqueous sodium hydroxide solution (0.5M, 120 mL) and aqueous layer was extracted with CH₂Cl₂ (3×100 mL) and the combined organic layers were washed with water (100 mL), brine (100 mL) dried over MgSO₄ and concentrated under vacuum. to give the product (2.25 g, 11.8 mmol, 91%) as a yellow liquid. The crude product was purified by CombiFlash Rfflash chromatography system equipped with an 24 g RediSep R_{f} silica gold column (gradient: 2 min 100% petrol ether then linear gradient to 100% petrol ether/EtOAc (4:3) over 12 min) to afford the product (647 mg, 2.90 mmol, 27%) as a pale yellow gum.

C₇H₇F₂NO₃S (223.2 g/mol).

### 2,2-Difluoro-2-(pyridin-2-ylthio)ethyl 4-methylbenzenesulfonate

Under nitrogen atmosphere a heat-gun dried round-bottom flask was charged with 2,2-difluoro-2-(pyridin-2-ylthio)ethan-1-ol (1.00 g, 5.23 mmol) and CH₂Cl₂ (20 mL). Then, the reaction mixture was cooled to 0° C. in an ice/water bath followed by addition of triethylamine (794 mg, 1.09 mL, 7.85 mmol), p-toluenesulfonyl chloride (1.50 g, 7.85 mmol) and the resulting mixture was stirred in the cooling bath for 16 hours. Afterwards the crude mixture was quenched by addition of aqueous hydrochloric acid solution (1M, 30 mL), diluted with CH₂Cl₂ (30 mL) and the organic layer was washed with brine (30 mL), dried over MgSO₄, concentrated under vacuum. The crude product was purified by CombiFlash Rfflash chromatography system equipped with an 24 g RediSep R_{f} silica gold column (gradient: 2 min 100% petroleum ether then linear gradient to 100% petroleum ether/EtOAc (2:1) over 12 min) to afford the product (1.60 g, 4.64 mmol, 89%) as a yellow solid.

C₁₄H₁₃F₂NO₃S₂ (345.4 g/mol).

### 2,2-Difluoro-2-(pyridin-2-ylsulfonyl)ethyl 4-methylbenzenesulfonate

A round-bottom flask was charged with 2,2-difluoro-2-(pyridin-2-ylthio)ethyl 4-methylbenzenesulfonate (7 g, 20.3 mmol), acetonitrile (100 mL), dichloromethane (50 mL), water (150 mL) and cooled 0° C. in an ice/water bath. Then, sodium periodate (21 g, 98.2 mmol) and ruthenium chloride hydrate (20 mg) were added to the reaction mixture and the resulting solution was stirred for 16 hours at room temperature. Afterwards the crude mixture was diluted with water (200 mL) and the aqueous mixture was extracted with EtOAc (3×250 mL). The combined organic layers were washed with water (100 mL), brine (100 mL), dried over MgSO₄, concentrated under vacuum and the crude product was purified by CombiFlash Rfflash chromatography system equipped with an 80 g RediSep R_{f} silica gold column (gradient: 2 min 100% petroleum ether then linear gradient to 100% EtOAc over 14 min to afford the product (7.66 g, 20.3 mmol, 100%) as a white solid.

C₁₄H₁₃F₂NO₅S₂ (377.4 g/mol).

### 2-((2-Azido-1,1-difluoroethyl)sulfonyl)pyridine

Under nitrogen atmosphere a heat-gun dried round-bottom flask was charged with 2,2-difluoro-2-(pyridin-2-ylsulfonyl)ethyl 4-methylbenzenesulfonate (1.51 g, 4.00 mmol), sodium azide (1.30 g, 20 mmol) and DMF (32 mL). After stirring for 133 hours at 70° C., the reaction mixture was cooled to room temperature, diluted with water (70 mL), extracted with EtOAc (3×70 mL), dried over MgSO₄ and concentrated under vacuum. The crude product was purified by CombiFlash Rfflash chromatography system equipped with an 40 g RediSep R_{f} silica gold column (gradient: 2 min 100% hexane then linear gradient to 100% petroleum ether:EtOAc (5:4) over 14 min to afford the product (650 mg, 2.62 mmol, 66%) as a white solid.

C₇H₆F₂N₄O₂S (248.2 g/mol).

### 2,2-Difluoro-2-(pyridin-2-ylsulfonyl)ethan-1-amine

Under nitrogen atmosphere a heat-gun dried round-bottom flask was charged with 2-((2-azido-1,1-difluoroethyl)sulfonyl)pyridine (650 mg, 2.62 mmol) and MeOH (20 mL). Then, triethylamine (448 mg, 617 μL, 4.43 mmol) and 1,3-propanedithiol (826 mg, 890 μL, 7.63 mmol) were added to the reaction mixture and stirred for two hours at room temperature. After concentration under vacuum, the crude product was purified by CombiFlash Rfflash chromatography system equipped with an 24 g RediSep R_{f} silica gold column (gradient: 3 min 100% CHCl₃ then linear gradient to 80% CHCl₃:MeOH (95:5) over 14 min to afford the product (520 mg, 2.34 mmol, 89%) as a pale yellow liquid.

C₇H₈F₂N₂O₂S (222.2 g/mol).

### N-(2,2-Difluoro-2-(pyridin-2-ylsulfonyl)ethyl)acetamide

Under nitrogen atmosphere a heat-gun dried round-bottom flask was charged with 2,2-difluoro-2-(pyridin-2-ylsulfonyl)ethan-1-amine (50 mg, 0.23 mmol), CH₂Cl₂ (1 mL) and DIPEA (101 mg, 136 μL, 0.78 mmol) followed by dropwise addition of acetic anhydride (29.5 mg, 27.2 μL, 0.29 mmol). After stirring for two hours at room temperature the reaction mixture was concentration under vacuum. Then, the crude mixture was dissolved in DCM (25 mL) and the resulting organic layer was washed with NaOH (2M, 10 mL), HCl (1M, 10 mL), brine (10 mL), dried over MgSO₄ and concentrated under vacuum. The crude product was purified by using a CombiFlash Rfflash chromatography system equipped with an 4 g RediSep R_{f} silica gold column (gradient: 2 min 100% CHCl₃ then linear gradient to 100% CHCl₃:MeOH (95:5) over 12 min and 5 min 100% EtOAc) to afford the product (48 mg, 0.18 mmol, 79%) as a pale yellow solid.

C₉H₁₀F₂N₂O₃S (264.3 g/mol).

### (2R,3S,4R,5R,6R)-5-Acetamido-2-(acetoxymethyl)-6-(2-bromo-2,2-difluoro-acetamido)-tetrahydro-2H-pyran-3,4-diyl diacetate

Under nitrogen atmosphere a heat-gun dried round-bottom flask was charged with (2R,3S,4R,5R,6R)-5-acetamido-2-(acetoxymethyl)-6-aminotetrahydro-2H-pyran-3,4-diyl diacetate (400 mg, 1.15 mmol), bromo difluoroacetic acid (242 mg, 1.38 mmol), EEDQ (341 mg, 1.38 mmol) and THE (16 mL) and the resulting mixture was stirred at room temperature. After 16 hours the crude product was concentrated under vacuum and purified by CombiFlash Rfflash chromatography system equipped with an 40 g RediSep R_{f} silica gold column (gradient: 1.5 min 100% CHCl₃ then linear gradient to 100% CHCl₃/MeOH (9:1) over 15 min) to afford the product (410 mg, 0.81 mmol, 71%) as a white solid.

C₁₆H₂₁BrF₂N₂O₉ (503.3 g/mol).

### N-((2R,3R,4R,5S,6R)-3-Acetamido-4,5-dihydroxy-6-(hydroxymethyl)tetrahydro-2H-pyran-2-yl)-2-bromo-2,2-difluoroacetamide

Under nitrogen atmosphere a heat-gun dried round-bottom flask was charged with (2R,3S,4R,5R,6R)-5-acetamido-2-(acetoxymethyl)-6-(2-bromo-2,2-difluoro-acetamido)-tetra-hydro-2H-pyran-3,4-diyl diacetate (250 mg, 0.50 mmol) and MeOH (5 mL). Then, a solution of sodium methoxide (25%, 114 μL) was added and the resulting reaction mixture was stirred for two hours at room temperature. Afterwards the crude mixture was quenched by addition of DOWEX H⁺ (100 mg) and stirred for 5 minutes. Finally, the reaction mixture was filtered, concentrated under vacuum to afford the product (185 mg, 0.49 mmol, 98%) as a white solid.

C₁₀H₁₅BrF₂N₂O₆ (377.1 g/mol).

### 2-((Trifluoromethyl)sulfonyl)pyridine

In a 25 mL round-bottomed flask was added pyfluor (2.60 mmol) and KHF₂ (2 mg, 0.26 mmol, 10 mol %) in DMSO (4 mL). To this mixture was added, TMSCF3 (384 μL, 2.60 mmol, 1.0 equiv). The reaction mixture was stirred for 30 minutes and then extracted into toluene (20 mL). Organic fractions were combined, dried over MgSO₄, filtered and concentrated in vacuo, to obtain the product in high purity as a pale yellow solid in 80% yield.

C₆H₄F₃NO₂S (211.0 g/mol).

### 2-((Fluoromethyl)sulfonyl)pyridine

To a solution of NaH (60%, wt %, 151 mg, 3.77 mmol, 1.05 equiv) and DMF (10 mL), was added in a dropwise fashion under a stream of N₂: pyridine-2-thiol (400 mg, 3.6 mmol, 1.0 equiv) dissolved in DMF (10 mL) at 0° C. CH₂FI (1.0 mL, 14.4 mmol, 4.0 equiv) (Note: CH₂FI is volatile and highly toxic) was then added dropwise over a period of 30 minutes. The reaction was then slowly allowed to warm to room temperature and was stirred overnight for 12 h. The reaction mixture was then quenched with H₂O (50 mL) and extracted with Et₂O (3×30 mL). The separated organic phase was then washed with Brine (50 mL) and dried over MgSO₄. The resulting solution was then filtered and concentrated in vacuo to afford crude 2-((fluoromethyl)thio)pyridine as a yellow oil. The crude product was then used without further purification in the next step. Crude 2-((fluoromethyl)thio)pyridine was added added to a 50 mL round-bottomed flask containing MeCN (10 mL), DCM (10 mL) and H₂O (20 mL). NaIO₄ (3.0 g, 14.5 mmol) and RuCl₃xH₂O (3 mg) were then subsequently added. The reaction was then monitored by ¹⁹F NMR until completion. Once complete, 10 mL of distilled H₂O was added, and the resulting reaction mixture was extracted with Et₂O (3×30 mL). The organic phase was then washed with saturated NaHCO₃ (30 mL) and brine (30 mL). The solution was then filtered and dried in vacuo. The crude residue was then subjected to silica gel chromatography (pentane/EtOAc, 3:1) to yield 2-((fluoromethyl)sulfonyl)pyridine as a colourless solid. Yield 55% (over two steps).

C₆H₆FNO₂S (175.1 g/mol).

### 2-((Fluoroiodomethyl)sulfonyl)pyridine

To a 100 mL pear-shaped schlenk tube were added under nitrogen, 2-((fluoromethyl)sulfonyl)pyridine (0.5 g, 2.9 mmol) and iodine crystals (1.46 g, 11.5 mmol, 4.0 equiv) in degassed anhydrous DMF (10 mL). To this mixture was subsequently added, tBuOK (1.1 g, 10 mmol, 3.5 equiv) in DMF (10 mL) at 5° C. The reaction was allowed to warm to room temperature and quenched with an aqueous saturated ammonium chloride solution (10 mL) when complete consumption of starting material was observed. The product was then extracted into EtOAc (3×20 mL) and stirred with aqueous NaHSO₃ (10 g, in 100 mL distilled water). ¹⁹F NMR was used to determine complete conversion of the diiodonated product (approximately 10 hours). The organic phase was then separated and washed with H₂O (2×30 mL) and brine (1×30 mL) and dried over MgSO₄. After filtration, the reaction mixture was concentrated in vacuo. The crude product was then subjected to column chromatography (EtOAc/pentane, 1:3), yielding 2-((fluoroiodomethyl)sulfonyl)pyridine in 62% yield as a white solid.

C₆H₅FNIO₂S (301.1 g/mol).

### 2-Fluoro-1-(4-methoxyphenyl)-2-(pyridin-2-ylsulfonyl)ethan-1-one

To a 100 mL pear-shaped schlenk tube were added under nitrogen, LiHMDS (24 mL, 1.0 M in THF, 24 mmol, 1.4 equiv) to a solution 2-((fluoromethyl)sulfonyl)pyridine (3.0 g, 17.1 mmol, 1.0 equiv) and methyl 4-methoxybenzoate (4.3 g, 25.7 mmol, 1.5 equiv) in 50 mL of THF at −78° C. The reaction mixture was then stirred for 30 minutes at this temperature. HCl_{(aq)} (3M, 15 mL) was then slowly added. The reaction mixture was then allowed to warm to room temperature. The organic phase was extracted with EtOAc (2×100 mL) and subsequently washed with distilled H₂O (100 mL) and brine (100 mL). The organic phase was then dried over MgSO₄, filtered and concentrated in vacuo. The crude product was then purified by silica gel chromatography (EtOAc/pentane, 1:3), yielding 2-fluoro-1-(4-methoxyphenyl)-2-(pyridin-2-ylsulfonyl)ethan-1-one as a white solid, 81% yield.

C₁₄H₁₂FNO₄S (309.0 g/mol).

### 2-((Chlorofluoromethyl)sulfonyl)pyridine

To a 25 mL pear-shaped schlenk tube were added under nitrogen, 2-fluoro-1-(4-methoxyphenyl)-2-(pyridin-2-ylsulfonyl)ethan-1-one (154 mg, 0.5 mmol, 1.0 equiv) and NCS (89 mg, 0.66 mmol, 1.3 equiv) in DMF (5 mL). The reaction mixture was cooled to −78° C. LiHMDS (0.75 mL, 1.0 M in THF, 0.75 mmol, 1.5 equiv) was then added dropwise over 10 minutes at −78° C. NaOH_{(aq)} (3 mL, 0.5 M) was then added and the reaction mixture was allowed to warm to room temperature. The organic phase was extracted with EtOAc (2×100 mL) and subsequently washed with distilled H₂O (100 mL) and brine (100 mL). The organic phase was then dried over MgSO₄, filtered and concentrated in vacuo. The crude product was then purified by silica gel chromatography (EtOAc/pentane, 1:3), yielding the title compound as a colourless oil, 70%.

C₆H₅ClFNO₂S (209.6 g/mol).

py-SOOF Biotin

NEt₃ (29 μL) was added to a solution of 3,3-difluoro-3-(pyridine-2-ylsulfonyl)propan-1-amine hydrogenchloride (23 mg, 84 μmol) and the active biotin ester (45 mg, 70 μmol) in anhydrous DCM under argon. The reaction mixture was left to stir overnight at rt and then concentrated. Purification by flash column chromatography (CHCl₃/MeOH 0->10% gradient elution) gave the title product as a white solid (25 mg, 50%).

C₂₉H₄₅F₂N₅O₉S₂ (709.8 g/mol).

### 2-((4-Methoxybenzyl)sulfonyl)pyridine

To a solution of 2-thiopyridine (1.11 g, 10 mmol) in MeCN (100 mL) was added PMB-Cl (1.62 mL, 12 mmol) followed by NEt₃ (2.09 mL, 15 mmol) dropwise. The reaction was stirred at room temperature for 2 h before being diluted with H₂O (150 mL) and neutralised to pH ˜7 with 2 M HCl. The mixture was then extracted with EtOAc (3×100 mL) and the combined organics were then washed with brine (100 mL), dried dried (MgSO₄), filtered and concentrated in vacuo. The crude yellow oil was then used without further purification.

The above crude oil was dissolved in CH₂Cl₂ (30 mL) and cooled to 0° C. mCPBA (4.5 g, 20 mmol) was then added portionwise. The mixture was then warmed to room temperature and stirred for 3 h before being quenched with a solution of saturated aqueous Na₂S₂O₃ (20 mL) and diluted with CH₂Cl₂ (70 mL) The organic phase was washed with saturated aqueous NaHCO₃ (3×60 mL), brine (70 mL), dried (MgSO₄), filtered and concentrated in vacuo. The crude product was then purified by flash chromatography (1:1 EtOAc:petroleum ether) to yield the desired pyridal sulfone as a white solid (1.51 g, 57% yield).

C₁₃H₁₃NO₃S (263.3 g/mol).

### 2-((Difluoro(4-methoxyphenyl)methyl)sulfonyl)pyridin

To a solution of sulfone (526 mg, 2 mmol) and NFSI (1.58 g, 5 mmol) in THE (80 mL) at −78° C. was added a solution of NaHMDS in THE (4.4 mL, 4.4 mmol, 1M). The mixture was stirred for at this temperature for 2.5 h, and then warmed to room temperature and stirred for 1.5 h. The reaction mixture was then cooled to 0° C. and quenched with saturated aqueous NH₄Cl (200 mL) and extracted with EtOAc (2×100 mL). The combined organic layers were then washed with saturated aqueous NaHCO₃ (200 mL), saturated aqueous NaCl (200 mL), dried (MgSO₄), filtered and concentrated in vacuo. The crude product was then purified by flash chromatography (1:1 EtOAc:petroleum ether) to yield the desired thioether as a yellow oil (487 mg, 80%).

C₁₃H₁₁F₂NO₃S (299.3 g/mol).

### 2-(Benzylthio)pyridine

PySH (2 g) was dissolved in 25 mL of anhydrous MeCN under argon. To this solution Et₃N (3.8 mL) was added. After 5 mins, benzyl bromide (2.65 mL) was added dropwise over 5 mins. After starting material was consumed (2h), the reaction was quenched with 1 M HCl. The mixture was partitioned between EtOAc and water, the aqueous phase extracted 3×with 20 mL EtOAc, dried with MgSO₄ and concentrated in vacuo. Half of the crude reaction was purified via flash chromatography on silica using hexane/EtOAc up to 8%. 1.34 g of pure product (37% regarding full stoichiometry) was obtained.

C₁₂H₁₀NS (201.3 g/mol).

### 2-(Benzylsulfonyl)pyridine

0.5 g of crude PySCH₂Ph was dissolved in 15 mL MeCN and 12 mL of DCM. To this mixture 20 mL of aqueous KIO₄ (5.75 g) suspension as well as 6 mg of RuCl₃ xH₂O was added and the reaction mixture was stirred overnight at rt. After that, the mixture was partitioned between DCM and water, separated and aqueous phase extracted 3×with 15 mL DCM. Combined organic fractions were filtered, dried with MgSO₄, filtered through silica plug and evaporated to dryness to yield 556 mg of brownish solid.

C₁₂H₁₀NO₂S (233.3 g/mol).

### 2-((Difluoro(phenyl)methyl)sulfonyl)pyridine

To a solution of sulfone (526 mg, 2 mmol) and NFSI (1.58 g, 5 mmol) in THE (80 mL) at −78° C. was added a solution of NaHMDS in THE (4.4 mL, 4.4 mmol, 1M). The mixture was stirred for at this temperature for 2.5 h, and then warmed to room temperature and stirred for 1.5 h. The reaction mixture was then cooled to 0° C. and quenched with saturated aqueous NH₄Cl (200 mL) and extracted with EtOAc (2×100 mL). The combined organic layers were then washed with saturated aqueous NaHCO₃ (200 mL), saturated aqueous NaCl (200 mL), dried (MgSO₄), filtered and concentrated in vacuo. The crude product was then purified by flash chromatography (1:1 EtOAc:petroleum ether) to yield the desired thioether as a yellow oil (487 mg, 80%).

C₁₂H₉F₂NO₂S (269.3 g/mol).

pySOOF-Arg Boc

To a solution of amine (50 mg, 0.23 mmol) in CH₂Cl₂ (2.3 mL) was added Goodman's guanidinylating reagent (88 mg, 0.23 mmol) followed by NEt₃ (32 mL, 0.23 mmol). The mixture was stirred at room temperature for 3 days and then diluted with CH₂Cl₂ (10 mL). The organic layer was then washed with 0.5 M HCl (3×10 mL) dried (MgSO₄), filtered and concentrated in vacuo. The crude product was then purified by flash chromatography (3:7 EtOAc:petroleum ether) to yield the desired protected guanidine as a white solid (83 mg, 77%).

C₁₈H₂₆F₂N₄O₆S (464.5 g/mol).

pySOOF-Arg

To a solution of diBoc-Guanidine (above) (35 mg, 0.075 mmol) in CH₂Cl₂ (1 mL) at 0° C. was added TFA (0.5 mL) slowly. The solution was stirred warming to room temperature over 1.5 h and then stirred for a further 1.5 h at room temperature. It was then concentrated in vacuo to yield the free guanidine as the TFA salt (30.2 mg, quant.) as a pale yellow oil.

C₈H₁₀F₂N₄O₂S (464.5 g/mol).

Catecholo-Ru(bpy)2

To a dried flask, catechol (30 mg, 0.27 mmol, 1.0 equiv) was dissolved in hot ethanol (2 mL). KOH (32 mg, 0.54 mmol, 2.0 equiv) was added, followed by cis-bis(2,2′-bipyridine)dichlororuthenium (II) hydrate (110 mg, 0.21 mmol, 0.80 equiv). The flask was fitted with a Dimroth condensor (though any might do), put under Ar and brought to reflux overnight. The mixture was cooled to room temperature, and ferrocenium hexafluorophosphate (69 mg, 0.27 mmol, 1 equiv) was added to ensure complete semiquinone formation. EtOH was removed and aqueous saturated KPF₆ was added to precipitate the complex. The solid material was dried overnight to obtain 290 mg of a very dark red solid. Full conversion by mass spectrometry was observed. The complex was further purified by dissolution in acetonitrile and application to silica column (10 g silica), eluted with 5% saturated aqueous KPF₆/acetonitrile to obtain 32 mg (48%) of a deep red solid (almost black). Thin-layer chromatography was used to follow the reaction (product R_{f}=0.64 in 5% aq. KPF₆/MeCN); the reactant/products have different shades of red and are visible by eye. High-resolution mass spectrometry (calculated 522.06243, observed 522.06256) confirmed the desired product.

General Experimental Protocol for BACED and pySOOF Reactions

All solutions were degassed for at least 8 h in a glovebox (<6 ppm O₂). Clear glass vials Chromacol 300 μL fixed insert vial, clear, screw top, Thermo Scientific for <100 μL, and 2 mL CLR RAM VIAL 9MM THD, 32009-1232, Novetech for >100 μL) with gas-tight caps (Cat. No VWRI548-3298, VWR) were used. Standard reactions consisted of mixing the Dha-containing protein of choice into the desired reaction buffer in the glovebox, followed by the sequential addition of catalyst, additive, and chemical substrates from stocks prepared fresh in buffer in the glovebox. Most reaction optimization and chemical substrate screening reactions were performed on model protein substrate X.I. Histone H3-Dha9 (1 mg/mL) in denaturing buffer (500 mM NH₄OAc, 3 M guanidinium chloride, pH 6.0) at a final concentration of 1 mg/mL (66 μM) in volumes of 50-200 μL. All reagents were first ported into a glovebox (<6 ppm O₂) where subsequent stock solutions and reactions would be prepared. All reagents were water soluble at their final concentrations and required no cosolvents unless explicitly noted. The reactions were then mixed thoroughly by pipette, capped, and removed from the glovebox for irradiation. 3 W blue (ca. 450 nm) LED flashlights were arranged for even irradiation of up to 20 reaction vials at a time or a variable intensity photobox was used for up to 7 reactions at a time with blue LED intensities ranging from 5-50 W (Intensity readings of 1-10 on the dial, respectively). Short reaction times (<20 min) did not result in significant temperature increases but longer reaction times (>20 min) could have their temperatures controlled by submerging the reaction vials in a glass beaker filled with water at the desired temperature. Irradiation proceeded for the desired time, afterwards which an aliquot of the reaction was diluted 25-fold for mass spectrometric analysis (2 μL in 48 μL water+0.1% formic acid) and conversions calculated relative to total ion counts of starting material, single, double addition, and any side reactions observed. Protein recovery was generally above 85% using PD SpinTrap G-25 (GE Healthcare) desalting columns and tracking overall protein absorbance, though this analysis was not performed for all conditions and substrates. The modified proteins could be stored in the freezer as a crude reaction mixture for several months without any degradation or appearance of new adducts, and in several cases, incomplete reactions could be continued simply by degassing the reaction mixture again and continuing with irradiation.

### Example 1—BACED Reactions

Reactions according to the methods of embodiments (ii) and (iii) described herein were demonstrated using a variety of substituents in order to functionalize example Dha containing proteins with a variety of different functional side chains.

All sidechains installed with the BACED reaction manifold (1a-1y, see FIG. 5) were screened on the model protein substrate Histone H3-Dha9. In many cases, more than one sidechain precursor substrate could be used to give the same sidechain product, such as potassium ethyltrifluoroborate or ethylboronic acid both giving the sidechain product 1a, for example. In these cases, all tested conditions leading to the same sidechain product are described. For the different histone variants, modification sites, or protein scaffolds, a variety of different sidechains were installed.

LC-MS/MS analysis was performed to confirm the site-specific sidechain installation. Conversions were calculated as a percentage of all products vs Dha starting material, based on the intensities of the deconvoluted LC/MS spectra. In some cases, minor undesired products such as double addition or catechol adducts were present, and are indicated as a percentage of the total product. As a general rule, a baseline cutoff of 10% was used when analyzing intensities of the deconvoluted spectra. In some cases, a small amount of methionine oxidation occurred during production, storage, and use (+16 Da+/−1 Da). These adducts were combined into the total sums for starting material and product calculations.
1a—In one example, ethyl was installed on a protein substrate using the BACED reaction manifold according to FIG. 6(A).

In the glovebox, a glass HPLC vial was charged with NH₄OAc buffer (500 mM, pH 6, 3M Gdn·HCl, 90 μL) containing Histone H3-Dha9 (100 g, final concentration of 1 mg/mL, 66 μM). After the sequential addition of Ru(bpy)₃Cl₂ (1 μL of a 66 mM stock prepared fresh in water, 10 eq), catechol (1 μL of a 660 mM stock prepared fresh in water, 100 eq) and potassium ethyltrifluoroborate (10 μL of a 330 mM stock prepared fresh in buffer, 500 eq), the vial was sealed with a cap, transferred out of the glovebox and irradiated with blue LED light (50 W) for 20 minutes. Conversion was determined by analysis of an aliquot of the crude mixture by LC-MS (82% conversion).

The same method was used to install a number of different groups onto protein substrates. The following table lists these further examples together with any variation in the reaction conditions. The resulting functionalized side chains are shown in FIG. 5.

| | | Starting | | Protein | Buffer | | | Irradiation | |
|---|---|---|---|---|---|---|---|---|---|
| | | material | | amount | volume | | Catechol | time | Conversion |
| Example | Starting material | eq | Protein | (μg) | (μL) | Catalyst | eq. | (mins) | (%) |
| 1a | potassium ethyltrifluoroborate | 500 | Histone | 100 | 90 | Ru(bpy)₃Cl₂ | 100 | 20 | 82 |
| | | | H3-Dha9 | | | | | | |
| 1a | ethylboronic acid | 500 | Histone | 100 | 90 | Ru(bpy)₃Cl₂ | 100 | 20 | 84 |
| | | | H3-Dha9 | | | | | | |
| 1b | 1-propylboronic acid catechol | 500 | Histone | 100 | 90 | Ru(bpy)₃Cl₂ | — | 15 | 100 |
| | ester | | H3-Dha9 | | | | | | |
| 1c | potassium | 500 | Histone | 100 | 90 | Ru(bpy)₃Cl₂ | 100 | 30 | 100 |
| | isopropyltrifluoroborate | | H3-Dha9 | | | | | | |
| 1c | isopropylboronic acid | 500 | Histone | 100 | 90 | Ru(bpy)₃Cl₂ | 100 | 15 | 83 |
| | | | H3-Dha9 | | | | | | |
| 1d | potassium | 500 | Histone | 100 | 90 | Ru(bpy)₃Cl₂ | 100 | 60 | 79 |
| | cyclohexyltrifluoroborate | | H3-Dha9 | | | | | | |
| 1e | potassium (3- | 500 | Histone | 50 | 45 | Ru(bpm)₃Cl₂ | 100 | 30 | 80 |
| | butenyl)trifluoroborate | | H3-Dha9 | | | | | | |
| 1f | 4-pentenylboronic acid¹ | 1000 | Histone | 50 | 45 | Ru(bpm)₃Cl₂ | 100 | 60 | 77 |
| | | | H3-Dha9 | | | | | | |
| 1g | potassium benzyltrifluoroborate | 500 | Histone | 100 | 90 | Ru(bpy)₃Cl₂ | 100 | 15 | 100 |
| | | | H3-Dha9 | | | | | | |
| 1g | benzylboronic acid pinacol | 500 | Histone | 100 | 90 | Ru(bpy)₃Cl₂ | 100 | 240 | 89 |
| | ester¹¹ | | H3-Dha9 | | | | | | |
| 1h | phenethylboronic acid¹⁰ | 500 | Histone | 100 | 90 | Ru(bpm)₃Cl₂ | 100 | 15 | 100 |
| | | | H3-Dha9 | | | | | | |
| 1h | potassium | 500 | Histone | 100 | 90 | Ru(bpm)₃Cl₂ | 100 | 15 | 100 |
| | phenethyltrifluoroborate¹⁰ | | H3-Dha9 | | | | | | |
| 1i | potassium | 500 | Histone | 100 | 90 | Ru(bpy)₃Cl₂ | 100 | 15 | 78 |
| | phenoxymethyltrifluoroborate¹ | | H3-Dha9 | | | | | | |
| 1j | potassium [(4- | 500 | Histone | 50 | 90 | Ru(bpy)₃Cl₂ | 100 | 60 | 79 |
| | methoxybenzyloxy)methyl]trifluoroborate² | | H3-Dha9 | | | | | | |
| 1k | 3-aminopropylboronic acid | 500 | Histone | 100 | 95 | Ru(bpm)₃Cl₂ | 100 | 60 | 85 |
| | pinacol ester | | H3-Dha9 | | | | | | |
| 1l | 3-trimethylaminopropylboronic | 500 | Histone | 100 | 95 | Ru(bpm)₃Cl₂ | 100 | 60 | 87 |
| | acid pinacol ester iodide | | H3-Dha9 | | | | | | |
| 1m | N-(3-(4,4,5,5-tetramethyl-1,3,2- | 250 | Histone | 100 | 90 | Ru(bpm)₃Cl₂ | 100 | 60 | 82 |
| | dioxaborolan-2- | | H3-Dha9 | | | | | | |
| | yl)propyl)acetamide | | | | | | | | |
| 1n | N-(3-(4,4,5,5-tetramethyl-1,3,2- | 1000 | Histone | 100 | 90 | Ru(bpm)₃Cl₂ | 100 | 60 | 85 |
| | dioxaborolan-2- | | H3-Dha9 | | | | | | |
| | yl)propyl)benzamide³ | | | | | | | | |
| 1o | 3-(3,3,4,4-tetramethylborolan-1- | 1000 | Histone | 100 | 90 | Ru(bpm)₃Cl₂ | 100 | 60 | 76 |
| | yl)propanoic acid | | H3-Dha9 | | | | | | |
| 1p | methyl 3-(3,3,4,4- | 500 | Histone | 100 | 90 | Ru(bpm)₃Cl₂ | 100 | 60 | 92 |
| | tetramethylborolan-1- | | H3-Dha9 | | | | | | |
| | yl)propanoate | | | | | | | | |
| 1q | tert-butyl 3-(3,3,4,4- | 1000 | Histone | 100 | 90 | Ru(bpm)₃Cl₂ | 100 | 60 | 88 |
| | tetramethylborolan-1- | | H3-Dha9 | | | | | | |
| | yl)propanoate | | | | | | | | |
| 1r | 3-azidopropylboronic acid | 1500 | Histone | 50 | 45 | Ru(bpm)₃Cl₂ | 100 | 60 | 84 |
| | pinacol ester | | H3-Dha9 | | | | | | |
| 1s | 3-iodopropylboronic acid | 1500 | Histone | 50 | 85 | Ru(bpm)₃Cl₂ | 100 | 60 | 76⁵ |
| | pinacol ester⁴ | | H3-Dha9 | | | | | | |
| 1t | 3-bromopropylboronic acid | 250 | Histone | 50 | 45 | Ru(bpm)₃Cl₂ | 100 | 60 | 81 |
| | pinacol ester | | H3-Dha9 | | | | | | |
| 1u | 4-bromobutylboronic acid | 1000 | Histone | 50 | 45 | Ru(bpm)₃Cl₂ | 100 | 60 | 86 |
| | | | H3-Dha9 | | | | | | |
| 1u | 4-bromobutylboronic acid | 500 | Histone | 50 | 45 | Ru(bpm)₃Cl₂ | 100 | 60 | 84 |
| | catechol ester | | H3-Dha9 | | | | | | |
| 1v | (4-(5-bromo-α-D- | 300 | Histone | 100 | 90 | Ru(bpm)₃Cl₂ | 100 | 60 | 86⁷ |
| | ribofuranose)butyl)boronic acid⁶ | | H3-Dha9 | | | | | | |
| 1w | (4-(5-chloro-α-D- | 150 | Histone | 100 | 90 | Ru(bpm)₃Cl₂ | 100 | 60 | 81⁷ |
| | ribofuranose)butyl)boronic acid⁶ | | H3-Dha9 | | | | | | |
| 1x | (2-acetamido-3-(benzylamino)- | 500 | Histone | 100 | 90 | Ru(bpm)₃Cl₂ | 100 | 30 | 85 |
| | 3-oxopropyl)boronic acid | | H3-Dha9 | | | | | | |
| 1y | biotin-PEG4-boronic acid | 250 | Histone | 50 | 90 | Ru(bpm)₃Cl₂ | 500 | 30 | 100 |
| | pinacol ester | | H3-Dha9 | | | | | | |
| 1r | 3-azidopropylboronic acid | 1400 | Human | 100 | 86 | Ru(bpm)₃Cl₂ | 100 | 60 | >90 |
| | pinacol ester | | Histone | | | | | | |
| | | | eH3-Dha18 | | | | | | |
| 1m | N-(3-(4,4,5,5-tetramethyl-1,3,2- | 250 | Human | 100 | 90 | Ru(bpm)₃Cl₂ | 100 | 60 | 90 |
| | dioxaborolan-2- | | Histone | | | | | | |
| | yl)propyl)acetamide | | eH3-Dha18 | | | | | | |
| 1n | N-(3-(4,4,5,5-tetramethyl-1,3,2- | 250 | Human | 100 | 90 | Ru(bpm)₃Cl₂ | 100 | 60 | 89 |
| | dioxaborolan-2- | | Histone | | | | | | |
| | y1)propyl)benzamide⁸ | | eH3-Dha18 | | | | | | |
| 1u | 4-bromobutylboronic acid | 500 | Human | 100 | 95 | Ru(bpm)₃Cl₂ | 50 | 60 | 72 |
| | | | Histone | | | | | | |
| | | | eH3-Dha4 | | | | | | |
| 1u | 4-bromobutylboronic acid | 500 | Human | 100 | 95 | Ru(bpm)₃Cl₂ | 50 | 60 | 80 |
| | | | Histone | | | | | | |
| | | | eH3-Dha9 | | | | | | |
| 1u | 4-bromobutylboronic acid | 500 | Human | 100 | 95 | Ru(bpm)₃Cl₂ | 50 | 60 | 81 |
| | | | Histone | | | | | | |
| | | | eH3-Dha27 | | | | | | |
| 1h | potassium | 1500 | Histone | 12.5 | 45 | Ru(bpy)₃Cl₂ | 300 | 30 | 89 |
| | phenethyltrifluoroborate⁹ | | H4-Dha16 | | | | | | |

¹660 mM starting material stock prepared fresh in 50:50 DMSO:H₂O.
²330 mM starting material stock prepared fresh in 3:1 buffer:DMSO.
³660 mM starting material stock prepared fresh in acetonitrile.
⁴660 mM starting material stock prepared fresh in DMSO.
⁵Conversion over three cycles.
⁶Dissolved in as a solid.
⁷Conversion over two cycles.
⁸280 mM starting material stock prepared fresh in acetonitrile.
⁹30 eq catalyst used.
¹⁰Buffer: Gdn · HCl concentration is 5M.
¹¹Irradiation power of 9 W used

Further examples of BACED reactions are set out below.

1h Installation—Large Scale

A glass vial containing pre-weighed quantities of Ru(bpm)₃Cl₂ (1.8 mg, 2.8 μmol), catechol (3.1 mg, 28 μmol), and 4-bromobutylboronic acid (76 mg, 420 μmol) was ported into a glovebox (<6 ppm O₂) and charged with NH₄OAc buffer (500 mM, pH 6, 3M Gdn·HCl, 2 mL) containing Human Histone H3-Dha9 (5 mg, final concentration of 2.5 mg/mL, 140 μM). After briefly missing with a pipette to solubilize the reagents, the vial was sealed with a cap, transferred out of the glovebox and irradiated with blue LED light (50 W) for 1 h. After the reaction, the solution was dialyzed thrice against milliQ H₂O (twice for 2 h, once overnight, 4° C.) and then nanodropped to determine the percent recovery of the protein (94%). Conversion was determined by analysis of an aliquot of the mixture post-dialysis by LC-MS.

1h Installation on AcrA-Dha123

In the glovebox, a glass HPLC vial was charged with fluorinated phosphate buffer (20 mM NaPi, 100 mM NaF, pH 7.4, 95 μL) containing AcrA-Dha123 (4 μM final concentration). After the sequential addition of Ru(bpy)₃Cl₂ (1 μL of a 4 mM stock prepared fresh in water, 10 eq), catechol (1 μL of a 20 mM stock prepared fresh in water, 50 eq) and potassium phenethyltrifluoroborate (5 μL of a 40 mM stock prepared fresh in buffer, 500 eq), the vial was sealed with a cap, transferred out of the glovebox and irradiated with blue LED light (50 W) for 15 minutes. Conversion was determined by analysis of an aliquot of the crude mixture by LC-MS. After the reaction, the sample was desalted (PD Minitrap G25) into the same buffer to remove excess reagents and analyzed by Circular Dichroism along with the relevant protein controls.

1h Installation on NPP-G2F-Dha61 In the glovebox, a glass HPLC vial was charged with fluorinated phosphate buffer (20 mM NaPi, 100 mM NaF, pH 7.4, 95 μL) containing NPP-G2F-M61Dha (40 μM final concentration). After the sequential addition of Ru(bpy)₃Cl₂ (1 μL of a 40 mM stock prepared fresh in water, 10 eq), catechol (1 μL of a 200 mM stock prepared fresh in water, 50 eq) and potassium phenethyltrifluoroborate (5 μL of a 400 mM stock prepared fresh in buffer, 500 eq), the vial was sealed with a cap, transferred out of the glovebox and irradiated with blue LED light (50 W) for 15 minutes. Conversion was determined by analysis of an aliquot of the crude mixture by LC-MS. After the reaction, the sample was desalted (PD Minitrap G25) into the same buffer to remove excess reagents and analyzed by Circular Dichroism along with the relevant protein controls.

1h Installation on PanC-Dha47

In the glovebox, a glass HPLC vial was charged with NH₄OAc buffer (500 mM, pH 6, 3M Gdn·HCl, 95 μL) containing PanC-Dha47 (4 μM final concentration). After the sequential addition of Ru(bpy)₃Cl₂ (1 μL of a 4 mM stock prepared fresh in water, 10 eq), catechol (1 L of a 20 mM stock prepared fresh in water, 50 eq) and potassium phenethyltrifluoroborate (5 μL of a 40 mM stock prepared fresh in buffer, 500 eq), the vial was sealed with a cap, transferred out of the glovebox and irradiated with blue LED light (50 W) for 15 minutes. Conversion was determined by analysis of an aliquot of the crude mixture by LC-MS.

### Example 2—ASOOF, Iodo-ASOOF and Difluorobromo Precursor Reactions

Reactions according to the methods of embodiments (i), (ia), and (ib) described herein were demonstrated using a variety of substituents in order to functionalize example Dha containing proteins with a variety of different functional side chains. The pySOOF reaction manifold was used as an exemplary ASOOF moiety according to embodiment (i).

All sidechains installed with the pySOOF reaction manifold (2a-2ag, see FIG. 5) were screened on the model protein substrate Histone H3-Dha9. This example also include the substrate scope originating from RC(O)CF₂Br (embodiment (ib)) radical precursors, as they follow the same mechanistic pathway. For the different histone variants, modification sites, or protein scaffolds, a variety of different sidechains were installed. LC-MS/MS analysis was used to confirm the site-specific sidechain installation. All reactions defined as “Large Scale” used >1 mg of protein Dha starting material and had their yields measured via Nanodrop after buffer exchanging to remove small molecule reaction components. All reactions were monitored via LC-MS. Conversions were calculated as a percentage of all products vs Dha starting material, based on the intensities of the deconvoluted LC/MS spectra. In some cases, minor undesired products such as double addition were present, and are indicated as a percentage of the total product. As a general rule, a baseline cutoff of 10% was used when analyzing intensities of the deconvoluted spectra. In many cases, a small amount of methionine oxidation occurred during production, storage, and use (+16 Da+/−1 Da). These adducts were combined into the total sums for starting material and product calculations.

2a—In a first example, —CF₂H was installed on a protein substrate using the pySOOF reaction manifold according to FIG. 6(B).

In the glovebox, a glass HPLC vial containing FeSO₄·7H₂O (408 g, 1.65 μmol) was charged with an aliquot of Histone H3-Dha9 (100 μg, 6.59 nmol) and diluted with NH₄OAc (500 mM, pH 6, 3M Gdn·HCl) to a final protein concentration of 1 mg/mL. After the addition of difluoromethyl 2-pyridyl sulfone (13.2 nmol in DMSO [0.02M]) and Ru(bpy)₃Cl₂ (16.48 nmol in 2 μL water), the vial was sealed with a cap, transferred out of the glovebox and irradiated with blue LED light (50 W) for 15 minutes. Conversion was determined by analysis of an aliquot of the crude mixture by LC-MS (conversion 100%). The same method was used to install a number of different groups onto protein substrates. The following table lists these further examples and sets out any variations in the reaction conditions. The resulting functionalized side chains are shown in FIG. 5.

| | | Starting | Starting | | | | |
|---|---|---|---|---|---|---|---|
| | | material | material | | FeSO₄•7H₂O | Catalyst | |
| | | amount | concentration | | amount | amount | Conversion |
| Example | Starting material solution | (nmol) | (M) | Protein | (μmol) | (nmol) | (%) |
| 2a | difluoromethyl 2-pyridyl | 13.2 | 0.02 | Histone | 1.65 | 16.48 | 100 |
| | sulfone in DMSO | | | H3-Dha9 | | | |
| 2b | 2-((1,1,1- | 329.5 | 0.02 | Histone | 1.32 | 65.9 | 80 |
| | trifluoromethyl)sulfonyl)pyridine in | | | H3-Dha9 | | | |
| | DMSO | | | | | | |
| 2c | 2-((1,1-difluoroethyl)sulfonyl)pyridine | 13.2 | 0.02 | Histone | 1.32 | 13.2 | 90 |
| | in DMSO | | | H3-Dha9 | | | |
| 2d | 2- | 32.95 | 0.02 | Histone | 0.66 | 13.2 | 87 |
| | ((difluoro(phenyl)methyl)sulfonyl)pyridine | | | H3-Dha9 | | | |
| | in DMSO | | | | | | |
| 2e | 2-((difluoro(4- | 65.9 | 0.02 | Histone | 1.65 | 33 | 89 |
| | methoxyphenyl)methyl)sulfonyl)pyridine | | | H3-Dha9 | | | |
| | in DMSO | | | | | | |
| 2f | 3,3-difluoro-3-(pyridin-2- | 13.2 | 0.02 | Histone | 0.659 | 13.2 | 100 |
| | ylsulfonyl)propan- | | | H3-Dha9 | | | |
| | 1-amine trifluoroacetate in DMSO | | | | | | |
| 2g | 2,2-difluoro-2-(pyridin-2- | 13.2 | 0.02 | Histone | 0.408 | 16.5 | 87 |
| | ylsulfonyl)ethan-1-amine in DMSO | | | H3-Dha9 | | | |
| 2h | 3,3-difluoro-N-methyl-3-(pyridin-2- | 13.2 | 0.02 | Histone | 0.659 | 13.2 | 100 |
| | ylsulfonyl)propan-1-aminium | | | H3-Dha9 | | | |
| | trifluoroacetate in DMSO | | | | | | |
| 2i | 3,3-difluoro-N,N-dimethyl-3-(pyridin- | 13.2 | 0.02 | Histone | 0.659 | 13.2 | 100 |
| | 2-ylsulfonyl)propan-1-amine in DMSO | | | H3-Dha9 | | | |
| 2j | 2-((1,1-difluoro-3-(trimethyl-l4- | 13.2 | 0.02 | Histone | 0.659 | 13.2 | 91 |
| | azaneyl)propyl)sulfonyl)pyridine iodide | | | H3-Dha9 | | | |
| | in DMSO | | | | | | |
| 2k | N-(3,3-difluoro-3-(pyridin-2- | 13.2 | 0.02 | Histone | 0.659 | 13.2 | 89 |
| | ylsulfonyl)propyl)acetamide in DMSO | | | H3-Dha9 | | | |
| 2l | N-(2,2-difluoro-2-(pyridin-2- | 13.2 | 0.02 | Histone | 1.65 | 16.5 | 91 |
| | ylsulfonyl)ethyl)acetamide in DMSO | | | H3-Dha9 | | | |
| 2m | tert-butyl (3,3-difluoro-3-(pyridin-2- | 32.9 | 0.02 | Histone | 0.659 | 13.2 | 100 |
| | ylsulfonyl)propyl)carbamate in DMSO | | | H3-Dha9 | | | |
| 2n | benzyl (3,3-difluoro-3-(pyridin-2- | 13.2 | 0.02 | Histone | 0.659 | 13.2 | 88 |
| | ylsulfonyl)propyl)carbamate in DMSO | | | H3-Dha9 | | | |
| 2o | 1-(2,2-difluoro-2-(pyridin-2- | 13.2 | 0.02 | Histone | 1.65 | 16.5 | 88 |
| | ylsulfonyl)ethyl)guanidine in DMSO | | | H3-Dha9 | | | |
| 2p | 2,2-difluoro-2-(pyridin-2- | 13.2 | 0.02 | Histone | 1.65 | 16.5 | 100 |
| | ylsulfonyl)ethan-1-ol in DMSO | | | H3-Dha9 | | | |
| 2q | 3,3-difluoro-3-(pyridin-2- | 13.2 | 0.02 | Histone | 0.659 | 13.2 | 100 |
| | ylsulfonyl)propan-1-ol in DMSO | | | H3-Dha9 | | | |
| 2r | 3,3-difluoro-3-(pyridin-2- | 13.2 | 0.02 | Histone | 0.659 | 13.2 | 91 |
| | ylsulfonyl)propyl acetate in DMSO | | | H3-Dha9 | | | |
| 2s | 2-fluoro-2-(pyridin-2- | 65.9 | 0.1 | Histone | 1.65 | 33 | 91 |
| | ylsulfonyl)acetamide in DMSO | | | H3-Dha9 | | | |
| 2u | 2-fluoro-2-(pyridin-2-ylsulfonyl)acetate | 165 | 0.1 | Histone | 1.65 | 33 | 89 |
| | in water | | | H3-Dha9 | | | |
| 2x | 2- | 13.2 | 0.02 | Histone | 1.65 | 16.5 | 100 |
| | ((difluoro(methylthio)methyl)sulfonyl)pyridine | | | H3-Dha9 | | | |
| | in DMSO | | | | | | |
| 2y | 2- | 13.2 | 0.02 | Histone | 1.65 | 16.5 | 87 |
| | ((difluoro(methylsulfinyl)methyl)sulfonyl)pyridine | | | H3-Dha9 | | | |
| | in DMSO | | | | | | |
| 2z | 2- | 13.2 | 0.02 | Histone | 1.65 | 16.5 | 81 |
| | ((difluoro(methylsulfonyl)methyl)sulfonyl)pyridine | | | H3-Dha9 | | | |
| | in DMSO | | | | | | |
| 2aa | 3,3-difluoro-3-(pyridin-2-ylsulfonyl)propyl | 13.2 | 0.02 | Histone | 1.65 | 16.5 | 89 |
| | hydrogen sulfate in DMSO | | | H3-Dha9 | | | |
| 2ab | 2-((2-azido-1,1- | 13.2 | 0.02 | Histone | 1.65 | 16.5 | 88 |
| | difluoroethyl)sulfonyl)pyridine in DMSO | | | H3-Dha9 | | | |
| 2ac | 2-((3-azido-1,1- | 13.2 | 0.02 | Histone | 1.65 | 16.5 | 91 |
| | difluoropropyl)sulfonyl)pyridine in DMSO | | | H3-Dha9 | | | |
| 2ad | 2-((1,1-difluoro-3- | 13.2 | 0.02 | Histone | 1.65 | 16.5 | 100 |
| | iodopropyl)sulfonyl)pyridine in DMSO | | | H3-Dha9 | | | |
| 2ag | N-(2-(3-((3,3-difluoro-3-(pyridin-2- | 13.2 | 0.1 | Histone | 0.66 | 26.4 | 87 |
| | ylsulfonyl)propyl)amino)-3-oxopropoxy)ethyl)- | | | H3-Dha9 | | | |
| | 5-(2-oxohexahydro-1H-thieno[3,4- | | | | | | |
| | d]imidazol-4-yl)pentanamide in DMSO | | | | | | |
| 2a | difluoromethyl 2-pyridyl sulfone in DMSO | 56.5 | 0.1 | Human | 1.41 | 28.3 | 100¹ |
| | | | | Histone | | | |
| | | | | eH3.1-Dha9 | | | |
| 2a | difluoromethyl 2-pyridyl sulfone in DMSO | 11.3 | 0.02 | Human | 1.41 | 14.1 | 100¹ |
| | | | | Histone | | | |
| | | | | eH3.1-Dha27 | | | |
| 2a | difluoromethyl 2-pyridyl sulfone in DMSO | 11.2 | 0.02 | Human | 1.41 | 14.1 | 78¹ |
| | | | | Histone | | | |
| | | | | eH3.1-Trp21- | | | |
| | | | | Dha9 | | | |
| 2a | difluoromethyl 2-pyridyl sulfone in DMSO ² | 17.9 | 0.02 | X.l Histone | 2.24 | 44.8 | 86 |
| | | | | H4-Dha16 | | | |
| 2a | difluoromethyl 2-pyridyl sulfone in DMSO | 119 | 0.06 | NPβ-Phe2- | 0.476 | 23.8 | 100 |
| | | | | Dha61³ | | | |
| 2a | difluoromethyl 2-pyridyl sulfone in DMSO ⁴ | 12.9 | 0.02 | AcrA-Dha123 | 0.219 | 6.45 | 100 |
| 2a | difluoromethyl 2-pyridyl sulfone in DMSO | 29.4 | 0.02 | PanC-Dha44 | 0.735 | 14.7 | 82 |
| 2a | difluoromethyl 2-pyridyl sulfone | 63.4 | 0.1 | cabLys3- | 3.17 | 31.7 | 100 |
| | | | | Dha101 | | | |

¹Crude reaction mixture incubated with EDTA (2 mg) for 15 min at room temperature prior to conversion analysis.
² Final protein concentration of 0.75 mg/mL.
³Diluted in NaPi (100 mM, pH 7) buffer.
⁴ Protein amount is 50 μg.

pySOOF reactions were also carried out on a large scale for a number of the starting materials, using essentially the same methods, except that the crude mixture was treated with EDTA (8 mg) followed by a buffer exchange using a PD midiTrap G25 to remove small molecule reagents. The protein concentration was then measured via Nanodrop to give a yield. Examples are presented in the table below.

| | | | Protein | Buffer | | |
|---|---|---|---|---|---|---|
| | | | amount | volume | Conversion | Yield |
| Example | Starting material | Protein | (mg) | (mL) | (%) | (%) |
| 2a | difluoromethyl 2-pyridyl sulfone | Histone | 4.6 | 1.5 | 100 | 90 |
| | | H3-Dha9 | | | | |
| 2f | 3,3-difluoro-3-(pyridin-2-ylsulfonyl)propan- | Histone | 3.8 | 1.5 | 100 | 98 |
| | 1-amine trifluoroacetate | H3-Dha9 | | | | |
| 2k | N-(3,3-difluoro-3-(pyridin-2- | Histone | 3.8 | 1.5 | 100 | 95 |
| | ylsulfonyl)propyl)acetamide | H3-Dha9 | | | | |
| 2x | 2-((difluoro(methylthio)methyl)sulfonyl)pyridine | Histone | 3.8 | 1.5 | 86 | 82 |
| | | H3-Dha9 | | | | |
| 2f | 3,3-difluoro-3-(pyridin-2-ylsulfonyl)propan- | Human | 1.5 | 0.5 | 100 | 96 |
| | 1-amine trifluoroacetate | Histone | | | | |
| | | eH3-Dha18 | | | | |
| 2k | N-(3,3-difluoro-3-(pyridin-2- | Human | 1.5 | 1.5 | >95 | 95 |
| | ylsulfonyl)propyl)acetamide | Histone | | | | |
| | | eH3.1-Dha18 | | | | |

pySOOF reactions were also carried out on a large scale for a number of the starting materials using essentially the same methods, except that after the reaction, beta-mercaptoethanol was added to a concentration of 80 mM, which was observed to have an advantageous effect in reducing extra methionine oxidation that was commonly observed when working with the FLAG-HA tagged Human Histone eH3. Examples are presented in the table below. 8

| | | | Protein | Buffer | |
|---|---|---|---|---|---|
| | | | amount | volume | Conversion |
| Example | Starting material | Protein | (mg) | (mL) | (%) |
| 2l | N-(2,2-difluoro-2-(pyridin-2- | Human Histone | 0.5 | 0.1 | 88 |
| | ylsulfonyl)ethyl)acetamide | eH3-Dha18 | | | |
| 2r | 3,3-difluoro-3-(pyridin-2-ylsulfonyl)propyl | Human Histone | 0.5 | 0.1 | 86 |
| | acetate | eH3-Dha18 | | | |
| 2i | 3,3-difluoro-N,N-dimethyl-3-(pyridin-2- | Human Histone | 0.5 | 0.1 | 89 |
| | ylsulfonyl)propan-1-amine | eH3-Dha18 | | | |
| 2j | 2-((1,1-difluoro-3-(trimethyl-l4- | Human Histone | 0.5 | 0.1 | 87 |
| | azaneyl)propyl)sulfonyl)pyridine iodide | eH3-Dha18 | | | |
| 2x | 2-((difluoro(methylthio)methyl)sulfonyl)pyridine | Human Histone | 0.5 | 0.1 | 85 |
| | | eH3-Dha18 | | | |
| 2y | 2-((difluoro(methylsulfinyl)methyl)sulfonyl)pyridine | Human Histone | 0.5 | 0.1 | 82 |
| | | eH3-Dha18 | | | |
| 2z | 2-((difluoro(methylsulfonyl)methyl)sulfonyl)pyridine | Human Histone | 0.5 | 0.1 | 88 |
| | | eH3-Dha18 | | | |

2t—In a further example the group —CF₂C(O)NH₂ was installed on a protein substrate using the difluorobromo radical precursors of embodiment (ib) according to FIG. 6(C).

In the glovebox, a glass HPLC vial containing FeSO4·7H2O (408 g, 1.65 μmol) was charged with an aliquot of Histone H3-Dha9 (100 g, 6.59 nmol) and diluted with NH4OAc (500 mM, pH 6, 3M Gdn·HCl to a final protein concentration of 1 mg/mL. After the addition of 2-bromo-2,2-difluoroacetamide (32.9 nmol in DMSO [0.02M]) and Ru(bpy)3Cl₂ (16.48 nmol in 2 μL water), the vial was sealed with a cap, transferred out of the glovebox and irradiated with blue LED light (50 W) for 15 minutes. Conversion was determined by analysis of an aliquot of the crude mixture by LC-MS. The same method was used to install a number of different groups onto protein substrates. The following table lists these further examples and sets out any variations in the reaction conditions. The resulting functionalized side chains are shown in FIG. 5.

| | | Starting | Starting | | | | |
|---|---|---|---|---|---|---|---|
| | | material | material | | FeSO₄•7H₂O | Catalyst | |
| | | amount | concentration | | amount | amount | Conversion |
| Example | Starting material solution | (nmol) | (M) | Protein | (μmol) | (nmol) | (%) |
| 2t | 2-bromo-2,2-difluoroacetamide in DMSO | 32.9 | 0.02 | Histone | 1.65 | 16.48 | 88 |
| | | | | H3-Dha9 | | | |
| 2v | sodium bromodifluoroacetate in DMSO | 329 | 0.02 | Histone | 1.65 | 33 | 100 |
| | | | | H3-Dha9 | | | |
| 2w | N-((2R,3R,4R,5S,6R)-3-acetamido-4,5- | 32.9 | 0.02 | Histone | 1.65 | 16.5 | 91 |
| | dihydroxy-6-(hydroxymethyl)tetrahydro-2H- | | | H3-Dha9 | | | |
| | pyran-2-yl)-2-bromo-2,2-difluoro-acetamide | | | | | | |
| | in water | | | | | | |

2ae—In a further example a mono fluorinated pySOOF group was installed on a protein substrate using the Iodo-pySOOF radical precursors of embodiment (ia) according to FIG. 6 (D).

In the glovebox, a glass HPLC vial containing FeSO4·7H₂O (408 μg, 1.65 μmol) was charged with an aliquot of Histone H3-Dha9 (100 g, 6.59 nmol) and diluted with NH₄OAc (500 mM, pH 6, 3M Gdn·HCl to a final protein concentration of 1 mg/mL. After the addition of 2-((fluoroiodomethyl)sulfonyl)pyridine (65.9 nmol in DMSO [0.1M]) and Ru(bpy)₃Cl₂ (33 nmol in 2 μL water), the vial was sealed with a cap, transferred out of the glovebox and irradiated with blue LED light (50 W) for 15 minutes. Conversion was determined by analysis of an aliquot of the crude mixture by LC-MS. The resulting functionalized side chains are shown in FIG. 5.

| | | Starting | Starting | | | | |
|---|---|---|---|---|---|---|---|
| | | material | material | | FeSO₄•7H₂O | Catalyst | |
| | | amount | concentration | | amount | amount | Conversion |
| Example | Starting material solution | (nmol) | (M) | Protein | (μmol) | (nmol) | (%) |
| 2ae | 2-((fluoroiodomethyl)sulfonyl)pyridine | 65.9 | 0.1 | Histone | 1.65 | 33 | 100 |
| | in DMSO | | | H3-Dha9 | | | |
| 2af | 2-((difluoroiodomethyl)sulfonyl)pyridine | 33 | 0.1 | Histone | 0.66 | 3.3 | 89 |
| | in DMSO | | | H3-Dha9 | | | |

### Example 3—on Protein Heterolytic Reactions

As shown above, the methods of the present invention, such as using an alkylhalide functionalized BACED reagent, allows proteins to be functionalized with highly reactive side chains such as alkyl halide side chains. Such electrophilic side chains allow for diverse further functinoalization, as shown in FIG. 3(b).

The scheme on FIG. 3(b) outlines a reaction scheme and LC/MS spectra for the installation of Bromonorleuccine (Bnl) and Iodonorleucine (Inl) through photoredox catalysis on boronate radical precursors. An investigation Bnl and Inl stability in a mildly acidic buffer revealed that the only reaction was slow halogen exchange of the Cl— ions for both I and Br, creating Chloronorleucine (Cnl). Both Bnl and Inl showed similar reactivity with Cl—, reaching full conversion after several days.

Manipulating pH or substrate equivalents further allows unwanted hydroxyl substitution and elimination side reactions to be disfavored, giving excellent conversions for the formation of C—S, C—P, and C—N bonds from on-protein alkyl halide reactive handles, as described below.

### Formation of Chloronorleucine (Cnl) at Mild pH

Reaction products from the installation of Iodonorleucine (Inl) and Bromonorleucine(Bnl), Histones H3-In19 and H3-Bnl9, were buffer exchanged immediately after the reaction into phosphate buffer (100 mM NaPi, 3 M Gdn·HCl, pH 6) to test their long term stability in a mildly acidic buffer. Samples of each modification (100 μL, 10 μM Histone H3-In19 or H3-Bnl9) were incubated at 37° C. with shaking (600 rpm), with aliquots from the crude reaction mixture taken for LC-MS analysis after 1, 16, 36, and 64 h. Analysis showed slow but nearly full conversion (˜90%) to the chloronorleucine containing product Histone H3-Cnl9, at roughly equal rates for both H3-In19 and H3-Bnl9, as well as little evidence of any other side reactions at any significant level (FIG. 3b).

Addition of βME to Histone H3-Inl/Bnl9

Reaction products from the installation of Inl and Bnl, Histones H3-In19 and H3-Bnl9, were buffer exchanged immediately after the reaction into phosphate buffer (100 mM NaPi, 3 M Gdn·HCl, pH 10). Neat PME was added to both samples (25 mM, 100 μL total reaction volume, 10 μM Histone H3-In19 or H3-Bnl9) and the samples were incubated at 37° C. with shaking (600 rpm) for 4 h. Aliquots from the crude reaction mixtures were taken for LC-MS analysis. Analysis showed full conversion for both Histone H3-In19 and H3-Bnl9, with βME substitution consisting of the major products in both cases and H3-Cnl9 formation (chloronorleucine formation from halogen exchange) as a minor product in both cases (FIG. 3b).

Addition of TCEP to Histone H3-Inl/Bnl9

Reaction products from the installation of Inl and Bnl, Histones H3-In19 and H3-Bnl9, were buffer exchanged immediately after the reaction into phosphate buffer (100 mM NaPi, 3 M Gdn·HCl, pH 10). TCEP was added (25 mM, from a 50 mM stock in buffer) to both samples (100 μL total reaction volume, 10 μM Histone H3-In19 or H3-Bnl9) and the samples were incubated at 37° C. with shaking (600 rpm) for 12 h. Aliquots from the crude reaction mixtures were taken for LC-MS analysis. Analysis showed full conversion for Histone H3-Inl9 and incomplete conversion for H3-Bnl9, with TCEP substitution consisting of the major products in both cases and H3-Cnl9 formation (chloronorleucine formation from halogen exchange) as a minor product in both cases (FIG. 3b).

Addition of Azide to Histone H3-Inl/Bnl9

Reaction products from the installation of Inl and Bnl, Histones H3-In19 and H3-Bnl9, were buffer exchanged immediately after the reaction into phosphate buffer (100 mM NaPi, 3 M Gdn·HCl, pH 10). Sodium azide was added (200 mM, from a 400 mM stock in buffer) to both samples (100 μL total reaction volume, 10 μM Histone H3-In19 or H3-Bnl9) and the samples were incubated at 37° C. with shaking (600 rpm) for 12 h. Aliquots from the crude reaction mixtures were taken for LC-MS analysis. Analysis showed full conversion for Histone H3-In19 and incomplete conversion for H3-Bnl9, with azide substitution consisting of the major products in both cases and H3-Cnl9 formation (chloronorleucine formation from halogen exchange) as a minor product for the reaction with H3-Bnl9. (FIG. 3b).

Addition of Methylamine to Histone H3-Inl/Bnl9

Reaction products from the installation of Inl and Bnl, Histones H3-In19 and H3-Bnl9, were buffer exchanged immediately after the reaction into phosphate buffer (100 mM NaPi, 3 M Gdn·HCl, pH 10). Methylamine was added (0.5 M, from a 1 M stock prepared in buffer from aqueous methylamine source) to both samples (100 μL total reaction volume, 10 μM Histone H3-In19 or H3-Bnl9) and the samples were incubated at 37° C. with shaking (600 rpm) for 12 h. Aliquots from the crude reaction mixtures were taken for LC-MS analysis. Analysis showed moderate conversion to the desired modification for both H3-In19 and H3-Bnl9, with methylamine substitution consisting of the major products in both cases but with significant amounts of minor products. The high pH required to deprotonate methylamine caused significant competition with the side-reactions discussed above and earlier (FIG. 3b), and only near molar equivalents of the reagent allowed for methylamine addition to exist as the major product (FIG. 3b).

Addition of Dimethylamine to Histone H3-Inl/Bnl9

Reaction products from the installation of Inl and Bnl, Histones H3-In19 and H3-Bnl9, were buffer exchanged immediately after the reaction into phosphate buffer (100 mM NaPi, 3 M Gdn·HCl, pH 10). Dimethylamine was added (0.5 M, from a 1 M stock prepared in buffer from the HCl salt) to both samples (100 μL total reaction volume, 10 μM Histone H3-In19 or H3-Bnl9) and the samples were incubated at 37° C. with shaking (600 rpm) for 1 h. Aliquots from the crude reaction mixtures were taken for LC-MS analysis. Analysis showed moderate conversion to the desired modification for both H3-In19 and H3-Bnl9, with dimethylamine substitution consisting of the major products in both cases but with moderate amounts of minor products. The high pH required to deprotonate dimethylamine caused some competition with the side-reactions discussed above (FIG. 3b), therefore near molar equivalents of the reagent were used to facilitate methylamine addition as the major product (FIG. 3b).

Addition of Trimethylamine to Histone H3-Inl/Bnl9

Reaction products from the installation of Inl and Bnl, Histones H3-In19 and H3-Bnl9, were buffer exchanged immediately after the reaction into phosphate buffer (100 mM NaPi, 3 M Gdn·HCl, pH 10). Trimethylamine was added (0.5 M, from a 1 M stock prepared in buffer from the an aqueous source) to both samples (100 μL total reaction volume, 10 μM Histone H3-In19 or H3-Bnl9) and the samples were incubated at 37° C. with shaking (600 rpm) for 1. Aliquots from the crude reaction mixtures were taken for LC-MS analysis. Analysis showed excellent conversion to the desired modification for both H3-In19 and H3-Bnl9, with trimethylamine substitution consisting of the major products in both cases but with a small amount of H3-Cnl9 formation present in the H3-Bnl9 reaction (FIG. 3b). Trimethylamine acts as an excellent nucleophile for both H3-In19 and H3-Bnl9, as side reactions are suppressed, even more so that with more than in the mono- or dimethylamine substitution reactions.

As shown in the examples above, flexibility (both structural and reactive) of the incorporated halogen electrophiles afforded a novel on-protein heterolytic reaction platform for conjugation with off-protein nucleophiles (FIG. 3B). This allowed, essentially, a strategic reversal (umpolung) of the common yet non-site-specific practice in the field of protein conjugation of using widely prevalent nucleophiles in proteins (Cys, Lys, etc.) to target off protein electrophiles. By tuning the pH, off-protein nucleophile concentration and halogen choice, it proved possible to selectively facilitate intermolecular nucleophile substitution at C-Hal bonds while avoiding putative, competing side-reactions of elimination and intraprotein nucleophile substitution. In addition to the creation of C—S(with thiol, beta-mercaptoethanol, BME), C—P bonds (with phosphine TCEP), and C—N bonds (with various methylamines creating methyllysine PTMs and N3—giving Anl), it was even possible to directly exchange halogens (Br→Cl or I→Cl) allowing further Finkelstein-type tuning of electrophile reactivity.

### Example 4—On-Protein Radical Reactions

As shown above, the methods of the present invention, can be used to functionalize proteins with on-protein radical precursor moieties such as the ASOOF motif Such groups allows for further diverse functionalization of the protein as shown in FIG. 3(a). Described below are various on-protein radical reactions which can be used to further functionalize the protein or peptide, e.g. via on-site radical polymerization, reactions with further radical substituents, and protein protein crosslinking.

General Protocol

In the glovebox, a glass HPLC vial was charged with an aliquot of Histone H3-pySOOF9 100 g, 6.59 nmol) and diluted with NH4OAc (500 mM, pH 6, 3M Gdn·HCl) to a final protein concentration of 1 mg/mL. After the addition of radical acceptor reagent (10-200 eq in DMSO [0.1M-0.5M] or water [0.1M-1M]), Ru(bpy)3Cl2 (2-5 eq in 2 μL water) and FeSO4·7H2O (0-100 eq in 4 μL water) the vial was sealed with a cap, transferred out of the glovebox and irradiated with blue LED light (50 W) for 15 minutes. Conversion was determined by analysis of an aliquot of the crude mixture by LC-MS. This work is summarized in FIG. 3a.

Reduction of pySOOF to DfeGly

In the glovebox, a glass HPLC vial was charged with an aliquot of Histone H3-pySOOF9 (100 g, 6.59 nmol) and diluted with NH4OAc (500 mM, pH 6, 3M Gdn·HCl) to a final protein concentration of 1 mg/mL. After the addition of Ru(bpy)3Cl2 (16.48 nmol in 2 μL water) and FeSO4·7H2O (1.648 μmol in 4 μL water) the vial was sealed with a cap, transferred out of the glovebox and irradiated with blue LED light (50 W) for 15 minutes. Conversion was determined by analysis of an aliquot of the crude mixture by LC-MS.

Installation of Vinyl Boronic Acid

In the glovebox, a glass HPLC vial was charged with an aliquot of Histone H3-pySOOF9 (100 g, 6.59 nmol) and diluted with NH4OAc (500 mM, pH 6, 3M Gdn·HCl) to a final protein concentration of 1 mg/mL. After the addition of vinyl boronic acid pinacol ester (1.318 μmol in DMSO [1M]) and Ru(bpy)3Cl2 (16.48 nmol in 2 μL water) the vial was sealed with a cap, transferred out of the glovebox and irradiated with blue LED light (50 W) for 15 minutes. Conversion was determined by analysis of an aliquot of the crude mixture by LC-MS.

Installation of N-acetyldehydroalanine

In the glovebox, a glass HPLC vial was charged with an aliquot of Histone H3-pySOOF9 (100 g, 6.59 nmol) and diluted with NH4OAc (500 mM, pH 6, 3M Gdn·HCl) to a final protein concentration of 1 mg/mL. After the addition of N-acetyldehydroalanine (0.824 mol in DMSO [0.5M]) and Ru(bpy)3Cl2 (26.36 nmol in 2 μL water) the vial was sealed with a cap, transferred out of the glovebox and irradiated with blue LED light (50 W) for 15 minutes. Conversion was determined by analysis of an aliquot of the crude mixture by LC-MS.

Installation of TEMPO

In the glovebox, a glass HPLC vial was charged with an aliquot of Histone H3-pySOOF9 (100 g, 6.59 nmol) and diluted with NH4OAc (500 mM, pH 6, 3M Gdn·HCl) to a final protein concentration of 1 mg/mL. After the addition of 4-hydroxy TEMPO (65.9 nmol in water [0.1M]), Ru(bpy)3Cl2 (13.18 nmol in 2 μL water) and FeSO4·7H2O (164.8 nmol in 2 L water) the vial was sealed with a cap, transferred out of the glovebox and irradiated with blue LED light (50 W) for 15 minutes. Conversion was determined by analysis of an aliquot of the crude mixture by LC-MS.

Installation of Diphenyl Diselenide

In the glovebox, a glass HPLC vial was charged with an aliquot of Histone H3-pySOOF9 (100 g, 6.59 nmol) and diluted with NH4OAc (500 mM, pH 6, 3M Gdn·HCl) to a final protein concentration of 1 mg/mL. After the addition of diphenyl diselenide (131.8 nmol in DMSO [0.1M]), Ru(bpy)3Cl2 (26.36 nmol in 2 μL water) and FeSO4·7H2O (164.8 nmol in 2 μL water) the vial was sealed with a cap, transferred out of the glovebox and irradiated with blue LED light (50 W) for 15 minutes. Conversion was determined by analysis of an aliquot of the crude mixture by LC-MS.

Installation of Boc-4-methylene-piperidine

In the glovebox, a glass HPLC vial was charged with an aliquot of Histone H3-pySOOF9 (100 g, 6.59 nmol) and diluted with NH4OAc (500 mM, pH 6, 3M Gdn·HCl) to a final protein concentration of 1 mg/mL. After the addition of Boc-4-methylene-piperidine (659 nmol in DMSO [0.5M]) and Ru(bpy)3Cl2 (32.95 nmol in 2 μL water) the vial was sealed with a cap, transferred out of the glovebox and irradiated with blue LED light (50 W) for 15 minutes. Conversion was determined by analysis of an aliquot of the crude mixture by LC-MS.

Installation of 3,4-butenediol

In the glovebox, a glass HPLC vial was charged with an aliquot of Histone H3-pySOOF9 (100 g, 6.59 nmol) and diluted with NH4OAc (500 mM, pH 6, 3M Gdn·HCl) to a final protein concentration of 1 mg/mL. After the addition of 3,4-butenediol (659 nmol in DMSO [0.5M]) and Ru(bpy)3Cl2 (32.95 nmol in 2 μL water) the vial was sealed with a cap, transferred out of the glovebox and irradiated with blue LED light (50 W) for 15 minutes. Conversion was determined by analysis of an aliquot of the crude mixture by LC-MS.

Installation of Vinyl Acetate

In the glovebox, a glass HPLC vial was charged with an aliquot of Histone H3-pySOOF9 (100 g, 6.59 nmol) and diluted with NH₄OAc (500 mM, pH 6, 3M Gdn·HCl) to a final protein concentration of 1 mg/mL. After the addition of vinyl acetate (659 nmol in DMSO [0.5M]) and Ru(bpy)3Cl2 (32.95 nmol in 2 μL water) the vial was sealed with a cap, transferred out of the glovebox and irradiated with blue LED light (50 W) for 15 minutes. Conversion was determined by analysis of an aliquot of the crude mixture by LC-MS.

Installation of Dimethyl Ethylidenemalonate

In the glovebox, a glass HPLC vial was charged with an aliquot of Histone H3-pySOOF9 (100 g, 6.59 nmol) and diluted with NH4OAc (500 mM, pH 6, 3M Gdn·HCl) to a final protein concentration of 1 mg/mL. After the addition of dimethyl ethylidenemalonate (659 nmol in DMSO [0.5M]) and Ru(bpy)3Cl2 (32.95 nmol in 2 μL water) the vial was sealed with a cap, transferred out of the glovebox and irradiated with blue LED light (50 W) for 15 minutes. Conversion was determined by analysis of an aliquot of the crude mixture by LC-MS.

Installation of Acrylamide

In the glovebox, a glass HPLC vial was charged with an aliquot of Histone H3-pySOOF9 (100 g, 6.59 nmol) and diluted with NH4OAc (500 mM, pH 6, 3M Gdn·HCl) to a final protein concentration of 1 mg/mL. After the addition of acrylamide (131.8 nmol in DMSO [0.1M]), FeSO4·7H2O (164.8 nmol in 2 μL water) and Ru(bpy)3Cl2 (32.95 nmol in 2 μL water) the vial was sealed with a cap, transferred out of the glovebox and irradiated with blue LED light (50 W) for 15 minutes. Conversion was determined by analysis of an aliquot of the crude mixture by LC-MS.

Functionalization with Further Dehydroalanine Containing Protein

A Histone H3 protein was functionalized with a further dehydroalanine containing protein, FLAG labelled eH3-Dha9, as set out in FIG. 6(E) The reaction was performed under the same conditions as set out for the above on-protein radical reactions, with the specific reagents and conditions set out in the reaction scheme below. The cross-linked protein-protein complex produced was confirmed with SDS Gel electrophoresis (see FIG. 3A).

### Example 5—KDM4A Crosslinking to Bhn Containing Histones

The initiation methods described above allow the insertion of varied, halogenated (chloro-, bromo- and iodo-), potentially electrophilic, side-chains into proteins including those with side-chain lengths precisely matched to Lys. This highlights the remarkable chemoselectivity and efficiency of the processes of the present invention through the use of reagents that not only contain moieties (alkylhalides) that have traditionally been used for 2e-heterolytic alkylation of protein-based nucleophiles but can also act as radical precursors through 1e-reductive initiation (see above); here they remained untouched during 1e-radical installation via C—C bond formation. When aliphatic 4-bromobutyl-boronic acid (precursor to the bromoalkyl sidechain bromohomonorleucine, Bhn, (1u) was evaluated using cyclic voltammetry under catechol-enhanced conditions (1:12) an irreversible oxidation event corresponding to half potential Eₒₓ=+0.93 V was observed. Notably, no reductive peak for C—Br activation was observed and no oxidation was observed in the absence of catechol, further confirming the benefit of BACED reagents. This control in the insertion of halogenated side chains into proteins allowed the design of site-selective ‘protein alkylators’ (FIG. 4C). These have the potential to remain essentially inactive under typical conditions in a biological mixture (FIG. 4C) but then display enhanced alkylative reactivity in a ‘guided’ manner by virtue of solvent exclusion, effective molarity and proper mimicry when suitably engaged and so ‘fitted’/tailored into a bound, protein-protein interface (PPI), (FIG. 4C). Such a system requires a balance in electrophilic reactivity and native shape fidelity, and therefore allows for investigation of protein protein interactions, e.g. enzyme substrate investigations.

The site-selective insertion of minimally-sized, alkylhalide side-chains such as bromonorleucine (Bnl) or bromohomonorleucine (Bhn) or even iodonorleucine (Inl) (FIG. 3B) into proteins has not been previously possible. Therefore, the methods of the present invention open up the potential for more closely mimicking the binding (and hence site-specific crosslinking) of specific sidechains with nucleophilic residues in interacting protein partners (FIG. 4D). In this way, Bhn or Bnl or Inl, by bearing the same simple alkyl-sidechain, represent near-direct (non-extended) alkyl halide mimics of Lys (FIG. 3A) allowing potentially for their probing, artefact-free, of even buried protein-protein interfaces in which Lys might reside in wild-type proteins. Residues of this type cannot be incorporated using, for example, complementary amber-codon suppression methods. To test this mimicry in a stringent, buried (and so space-limited) and transient (substrate·enzyme) PPI, bromohomonorleucine (Bhn, 1u) was installed as a brominated mimic of Lys at three sites in human histone isoform H3.1 (C-terminally FLAG-HA tagged form, eH3.1) that are normally occupied by Lys (sites 4, 9 and 27) to create eH3.1-Bhn4, eH3.1-Bhn9 and eH3.1-Bhn27, respectively. These ‘guided alkylator proteins’ and a WT control were incubated with a representative partner enzyme that processes, and so binds, Lys residues, the human histone Lys-demethylase KDM4A (N-terminally His-tagged, FIG. 4D). Coomassie staining and Western blots showed crosslinking exclusive to the mixtures with KDM4A and Bhn-containing histone H3 proteins, but not with WT histone H3 (FIG. 4D). The ‘guided’ nature of this cross-linking was confirmed by incubations with control proteins. None of the Bhn-containing histones showed any evidence of cross-linking with either serum albumin (bovine, BSA, as a known Cys-rich control) or with known nucleosomal binding partner histone H4 (which non-covalently forms H3/H4 dimers and tetramers and is Cys-free), even after extended periods and elevated temperature (2 h at 37° C.). Notably, the H3·H4 PPI does not involve Lys4, Lys9 or Lys27,64 whereas only the H₃KDM4A PPI does. Together, this lack of reaction with BSA or H4, despite their potential for non-specific reaction and/or binding suggests that observed cross-linking of our edited functionalized H3.1-Bhn variants requires a suitable PPI such as in KDM4A.

This seeming PPI-selective reaction was confirmed by MS/MS analysis (FIG. 4D) that revealed highly conserved crosslinking in KDM4A to the two cysteines (Cys234 and Cys306) located at the Zn-binding domain in the critical H3·KDM4A PPI interface pocket (FIG. 4C). Bhn therefore, by virtue of its near-direct (non-extended) structural analogy to Lys is a representative mimic of Lys behaviour—covalently probing, artefact-free, the same protein-protein interfaces as Lys when ‘edited’ inserted into relevant Lys sites in a protein. In this way, Bhn in the H3-Lys→Bhn ‘mutants’ created ‘reach to’ the same sites in the confined PPI of the H3·KDM4A complex as the corresponding H3-Lys wild-type proteins.

The reaction of the Bhn side chain in eH3.1-Bhn with KDM4A was directly and quantitatively assessed through zinc ejection studies (FIG. 4D).

The ability of alkylator proteins to capture interaction partners was investigated with dual-FLAG+HA tagged histone eH3.1-Bhn9, which was immobilized onto beads bearing anti-HA-flag antibodies and incubated with human cell (HeLa) nuclear lysate (4 h, 37 C) to facilitate the capture of interaction partners of eH3.1-Lys9 present in cells. After such capture, western blots (anti-FLAG to selectively detect any eH3.1-adduct species) revealed the presence of several distinct eH3.1-adduct species found only in samples containing alkylator protein Histone eH3.1-Bhn9 (bearing sidechain 1u at site 9), whilst wildtype Histone eH3.1 and control without histone showed none (FIG. 4E).

The retained inherent reactivity of eH3 proteins with high concentration small molecules; the failure of KDM4A to react with low concentration small-molecule side-chain reagent; and the successful reaction of eH3-Bhn with KDM4A even at low (nM-μM) levels confirmed the origin of this novel ‘effective molarity-driven’ cross-linking reaction (again at levels of EM >103).

Histone eH3-Bhn-KDM4A Crosslinking General Protocol

The histone modifying enzyme KDM4A (2 μM) was mixed with either the modified histone eH3.1-Bhn4/9/27 or the WT control (4 μM) in HEPES buffer (50 mM, pH 7.4) and incubated at the indicated temperature and for the indicated time. The crosslinking reaction was quenched with the addition of 5×Laemmli buffer and analyzed via SDS-PAGE or Western Blot (see FIG. 4E).

Antibody details for western blot analysis DYKDDDDK (FLAG) Tag Monoclonal Antibody (eBioscience, catalogue number 14-6681-82, clone FG4R, lot number 1981531, dilution 1:1,000), Monoclonal Anti-polyHistidine-Alkaline Phosphatase (Sigma-Aldrich, catalogue number A5588, clone HIS-1, lot number 085M4836V, dilution 1:2,000), Histone H3 Antibody (Cell Signaling Technology, catalogue number 3638S, clone 96C10, lot number 10, 1:1,000), Goat Anti-Mouse IgG H&L Alkaline Phosphatase (Sigma-Aldrich, catalog number A3562, polyclonal, lot number SLCB8722, dilution 1:10,000), Anti-Mouse IgG (H+L) HRP Conjugate (Promega, catalogue number W4021, polyclonal, lot number 0000306114, dilution 1:2,500). All antibodies were used per the manufacturers' instruction.

Zn Ejection Assay

Zn ejection assay was performed using N-(6-nethoxy-8-quinolyl)-p-toluenesulfonamide (TSQ) (Enzo) Zn(II) fluorophore as described with minor modifications28,33. In brief, assays were performed in 384 well black μCLEAR® non-binding plates (Grenier) using a reaction volume of 100 μL at 37° C. on a BMG CLARIOstar (360ex/490em). The plate was shaken (5 s, 700 rpm) before each reading, taken every 22 s for 270 cycles. Reactions consisted of 10 M TSQ, 25 μM Ebselen or 20 μM H3 K9Bhn/H3-wt/4-bromobutylboronic acid, and those with enzyme contained 2 μM KDM4A all with 1.1% (v/v) DMSO in 50 mM HEPES (pH 7.5). Compounds and TSQ were added to the plate before initiating the assay with addition of KDM4A using the CLARIOstar injector (FIG. 4d). An internal calibration curve of ZnCl2 (0-2 μM) in 50 mM HEPES (pH 7.5) was included in each experiment to quantitate the concentrations of Zn(II) ejected. Data were normalised by subtracting a no enzyme control for that compound at each time point. Mean±standard deviation (n=3 technical replicates) was plotted for each time point using GraphPad Prism 5.0, representative data from three biological replicates is shown.

MS/MS data suggests that Histone eH3-9Bhn cross-links to a Cys3-His Zn(II) binding site close to the active site. The rate of Zn(II) ejection was calculated from the slope of linear regression plotted over the linear region of Zn(II) ejection (from 946-3982 s) plotted in GraphPad Prism 5.0. Time-dependent release of Zn(II) from KDM4A was observed (9.270±0.025 nM/min) when incubated with eH3-Bhn9 but not with unmodified eH3 or 4-bromobutylboronic acid (FIG. 4d). This contrasts with the rapid Zn(II) ejection rate with Ebselen, a Zn(II) chelating small molecule inhibitor of KDM4A activity28, at >1663 nM/min. This suggests that the rate of release of Zn(II) is dependent on the rate of H3-Bhn9 cross-linking to KDM4A.

### Example 6—Effective Molarity-Driven Cross-Linking Reactions

The enhancement of nucleophilicity by “effective molarity” provided by protein protein interfaces was further demonstrated by the unprecedented formation of a Williamson-type (—C—O—C—) ether (FIG. 4F). The second order rate constant of this type of reaction has always been considered to be far too low (kₐₚₚ<10⁻⁴ M·s⁻¹) to allow effective cross-linking of protein-protein interactions at low (nM-μM) protein concentrations. The formation of an inter (and not intra) CP—O—CH₂—Bhn4 ether link therefore implies a strongly EM-enhanced protein-protein interaction of one H3 protein with another. This is considered to be due to the presence of a transient H3·H3 dimer in the presence of KDM4A.

This demonstrates the potential of the present methods to functionalize proteins to contain precisely mimicking residues such as Bhn that can trap transient intermediates and so provide information on new speculative mechanistic models.

Protein Partner Binding

To further investigate the ability of such alkylator proteins to capture interaction partners, dual-FLAG+HA tagged histone eH3.1-Bhn9 was immobilized onto beads bearing anti-HA-flag antibodies and incubated with human cell (HeLa) nuclear lysate (4 h, 37 C) to facilitate the capture of interaction partners of eH3.1-Lys9 present in cells as set out below, demonstrating the presence of various protein interaction partners.

Histone samples (20 μg or either Human Histone eH3.1-WT, Human Histone eH3.1-Bhn9, or no Histone control) were immobilized on Anti-HA Magbeads (Pierce 88836, 50 μL/sample pre-equilibrated in buffer used for immobilization) via their HA epitope tag for 30 min at RT in HEPES buffer (50 mM, pH 7.5). The beads were then incubated with HeLa nuclear lysate (250 μL, 0.5 mg/mL, 4 hr, 37° C., 600 rpm) to promote the crosslinking. The HeLa nuclear lysate was prepared as previously described2. After incubation, the beads were washed (5×with 500 μL HEPES buffer+0.1% Tween20, 1×with sdH2O). The histones+interaction partners were eluted off the beads with Glycine (0.1 M, pH 2.0, 100 μL, 10 min, 37° C.) and quenched with Tris buffer (1 M, pH 8.5, 15 μL). The elution and quenching was repeated once more with the beads. To analyse the crosslinking and immunoprecipitation, controls of all histones and lysate, as well as samples from all conditions for incubation, last wash, and elution were checked via SDS-PAGE with either a Coomassie Blue staining or by western blot with an α-FLAG antibody (Histone eH3 samples are FLAG-HA epitope tagged) to detect higher MW bands corresponding to the mass of the Histone eH3.1 covalently crosslinking to an unknown interaction partner (See FIGS. 4C and 4E).

### Example 7—Investigating Reaction Mechanisms

Insertion of native and “Zero-Size”-labeled and reactive sidechains into proteins further allowed insight into enzymes that post-translationally modify them.

Lys mimicry (FIG. 4) was tested through the installation of acetyl- (AcLys/KAc, 1 m) and benzoyl-lysine (BzLys/KBz, In) side-chains as well as H→F labeled side chain analogues K[γF₂]Ac 2 k and K[γF₂] 2f into protein precursors, respectively.

H3-KAc18 and H3-KBzl8 were generated using BACED reagents (FIG. 4A). These proteins enabled timecourse studies during the incubation of Sirt2 with both histone H3-K18Ac and H3-K18Bz which confirmed56 true Sirt2 activity on both acylated Lys an revealed a strong, substrate KAc >KBz selectivity by Sirt2 (FIG. 4A).

The pySOOF reagents were also used to generate corresponding H—F labeled side chain analogues such as K[γF₂]Ac and K[γF₂] sidechains 2 k and 2f, respectively. The centrally-placed γ-carbon-F₂ label in these systems proved to be powerful in enabling in situ reporting of the modification state of these sidechains. Alteration of the sidechain identity at position 18 in human H3.1 could be detected simply by use of protein ¹⁹F NMR (565 MHz) that sensitively distinguished identity of H3.1-K18 from →H3.1-KAc18 despite the 4 or 5-bond distance from γ-carbon-F₂ label to the sites of change, hence probing modification state (sidechains 2f→2 k=δF −98.0→−99.4, FIG. 4B). Other sidechain variations could similarly be distinguished at different sites in the same protein e.g. H3.1-K9→H3.1-KAc9→H3.1-Kme39 (sidechains 2f→2 k→2j=δF −99.0→−98.0→−99.2) or H3.1-M27 (sidechain 2×δF −74.8) or H3.1-E9 (sidechain 2u δF −103.3). In this way, the diverse scope of available further sidechains allows this approach to be explored in numerous additional directions such as e.g. monitoring heteroatom variation (e.g. N→O, ‘deaza-oxo’ variant KOAc, sidechain 2r for H3.1-K18→H3.1-KOAc18, sidechains 2 k→2r) or even precisely assaying sidechain Met oxidation state (H3.1-M27→H3.1-Mₒₓ27→H3.1-Mₒₓ27, sidechains 2x→2y→2z).

Due to the site-selectivity of the insertion of this label and its excellent sensitivity in ‘zero background’, not only could chemical shift of ¹⁹F signal be ‘read’ but also its multiplicity through correlated simulation (FIG. 4B). In this way the γ-F₂ label could simultaneously report on both the processing of sidechain modification (KAc→K at the NE site, 5 bonds ‘down’ the sidechain) but also, by virtue of highly sensitive CF₂-diastereotopicity, the stereo-chemical processing (and hence selectivity L vs D at the Cα site, 3 bonds ‘up’ the sidechain). This remarkable sensitivity along the full length of the residue sidechain in turn allowed, in situ, on-protein reporting of enzyme-mediated post-translational modification in real-time—this revealed that the HDAC deacylation enzyme Sirt2 (despite its processing of a modification that is six bonds distant) shows a L/D selectivity preference of >14 (ΔΔGø>6.6 kJ mol⁻¹).

Thus, the insertion of the site specific labels of the present invention further allow simultaneous, real-time determinations of both substrate- and stereo-selectivity for post-translation-modifying enzymes in intact proteins which has not been previously possible. The sensitivity of the γ-F₂ label was applied to monitor differential folding and higher assembly states in a single protein. Thus, use of H3-DfeGly9 allowed the full step-wise processes of histone octamer assembly to be monitored directly at each step from unfolded H3 monomer→folded H3 monomer→(H3)₂·(H4)₂ hetero-tetramer to full (H3)₂·(H4)₂° (H2A)₂·(H2B)₂ hetero-octamer, even at low sub-milligram scales.

Octamer Reconstitution for ¹⁹F-NMR Measurement

After ¹⁹F-NMR measurement of the unfolded Histone H3-DfeGly9, 2 mg of the Histone protein was buffer exchanged into 1 mL of Unfolding Buffer (7 M Gdn·HCl, 10 mM Tris, 1 mM EDTA, 10 mM DTT, 1 mM Benzamidine, pH 7.5) then buffer exchanged into Tris Buffer (150 mM NaCl, 10 mM Tris, 1 mM EDTA, 2 mM PME, pH 7.5) with a PD10 G-25 Minitrap. An equivalent volume of Deuterated Tris Buffer (as above but made with 100% D₂O) to make a final buffer with 50% D₂O, which was concentrated to a volume of 0.75 mL (Vivaspin 6, 5 kDa MWCO). The mixture was centrifuged (15000 rpm, 10 min, 4° C.) to pellet any precipitant, and an internal standard of trifluoroethanol (0.001 μL) was added. The concentration was measured (Nanodrop, 2.0 mg/mL), folding checked via Circular Dichroism (CD), and filtered into an NMR tube.

For the Histone H3-DfeGly9-H4 tetramer reconstitution, the modified Histone H3 and Histone H4 WT (1:1 molar ratio, 2.5 mg of Histone H3-DfeGly9) were mixed in Unfolding Buffer (6 mL), incubated for 30 min at RT, then dialyzed into Refolding Buffer (3×into 1 L, 2 hr each with one overnight). The resultant solution was centrifuged (15000 rpm, 10 min, 4° C.) to pellet any precipitant, concentration was measured (0.5 mg/mL, 1 mL), and purified via Size Exclusion (Superdex S75, 16/60 pre-equilibrated in Refolding Buffer). Tetramer containing fractions (visualized via SDS-PAGE analysis) were combined, concentrated and resuspended into 50% Deuterated Refolding Buffer (made with 1:1 H₂O/D₂O). Trifluoroethanol (0.1 μL in 1 mL) was added as an internal NMR standard. The final concentration was measured (Nanodrop, 2.5 mg/mL), and folding checked via Circular Dichroism (CD) before filtering into an NMR tube.

For the Histone H3-DfeGly9-H4-H2A-H2B octamer reconstitution, all histones were dissolved in Unfolding Buffer (1:1:1.1:1.1 molar ratio, 25 nmol of modified Histone H3) and incubated for 30 min at RT before dialyzing into Refolding Buffer (3×into 1 L, 2 hr each with one overnight). The resultant solution was centrifuged (15000 rpm, 10 min, 4° C.) to 310 pellet any precipitant before purifying via Size Exclusion as above. Fractions containing H3F-H4-H2A-H2B Octamer were collected, concentration measured (Nanodrop, 0.8 mg total), an internal trifluoroethanol standard was added (0.1 μL) and the NMR sample was prepared and measured as above. After the NMR, the octamer was analysed by SDS-PAGE and CD to check proper folding.

### Further Synthesis Examples

Further compounds used in the examples were synthesised as set out below. The reaction products were analysed and confirmed with ¹H, ¹³C and ¹⁹F NMR.

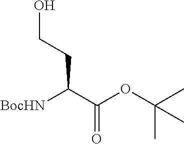 [SMILES: CC(C)(C)OC(=O)N[C@@H](CCO)C(=O)OC(C)(C)C]

To a solution of aspartic acid (5.0 g, 17.3 mmol) in THE (170 mL) at 0° C. was added iso-butyl chloroformate (6.8 mL, 51.8 mmol) followed by iPr₂NEt (4.5 mL, 25.95 mmol) and stirred at 0° C. for 2 h. NaBH₄ (4.58 g, 121.3 mmol) was added portionwise before careful addition of H₂O (40 mL) over about 30 min. The mixture was then warmed to room temperature and then quenched with saturated aqueous NH₄Cl (300 mL) and extracted with EtOAc (3×200 mL). The combined organic layers were washed with saturated aqueous NaCl (300 mL), dried (MgSO₄), filtered and concentrated in vacuo to yield a the alcohol as a yellow oil. The crude product was then purified by flash chromatography (3:7, EtOAc:petroleum ether) to yield the desired difluoro sulfone as a colourless oil (4.3 g, 90° % yield) AMG-1-48-A

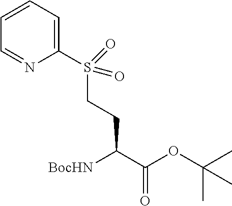 [SMILES: CC(C)(C)OC(=O)N[C@@H](CCS(=O)(=O)c1ccccn1)C(=O)OC(C)(C)C]

To a solution of the alcohol (2.0 g, 7.24 mmol) in CH₂Cl₂ (50 mL) at 0° C. was added triethylamine (2.5 mL, 18.15 mmol) followed by slow addition of mesylchloride (670 μL, 8.7 mmol). The mixture was stirred at this temperature for 30 min before being poured onto saturated aqueous NaCl (150 mL). The aqueous phase was extracted with CH₂Cl₂ (3×100 mL) and the combined organic layers were dried (MgSO₄), filtered and concentrated in vacuo to afford the mesylate as white needles.

To a solution of the crude mesylate in MeCN (50 mL) was added 2-thiopyridine (968 mg, 8.71 mmol) and triethylamine (1.52 mL, 10.52 mmol). The reaction mixture was stirred for 72 h before being quenched with H₂O (100 mL) and adjusted to pH ˜ 7 with 1M HCl. Then aqueous phase was then extracted with EtOAc (3×70 mL) and the combined organic layers were dried (MgSO₄), filtered and concentrated in vacuo to yield a yellow oil.

To a solution crude thioether in CH₂Cl₂ (50 mL) at 0° C. was added mCPBA (3.56 g, 15.97 mmol, 77% by weight). The mixture was stirred at this temperature for 3 h before being quenched with 10% aqueous Na₂S₂O₃ (100 mL) and extracted with CH₂Cl₂ (2×50 mL). The combined organic phases were washed with saturated aqueous NaHCO₃ (3×150 mL), dried (MgSO₄), filtered and concentrated in vacuo. The crude product was then purified by flash chromatography (7:13, EtOAc:petroleum ether) to yield the desired sulfone as a white solid (2.02 g, 69% yield from starting alcohol) AMG-1-64-A

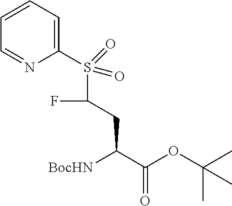 [SMILES: CC(C)(C)OC(=O)N[C@@H](CC(F)S(=O)(=O)c1ccccn1)C(=O)OC(C)(C)C]

To a solution of the sulfone (1.25 g, 3.21 mmol) and NFSI (1.37 g, 4.27 mmol) in THE (45 mL) at −78° C. was added dropwise a solution of NaHMDS in THE (7.49 mL, 1M). The solution was stirred at this temperature for 4.5 h before being quenched with saturated aqueous NH₄Cl (150 mL). The aqueous phase was then extracted with EtOAc (3×100 mL) and the organic phase was dried (MgSO₄), filtered and concentrated in vacuo. The crude product was then purified by flash chromatography (5:95, EtOAc:CH₂Cl₂) to yield the desired sulfone (contaminated with ˜8% diF compound) as a white solid (740 mg, 57% yield from starting alcohol)) AMG-2-20-A AMG-3-05

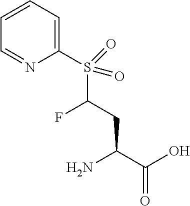 [SMILES: N[C@@H](CC(F)S(=O)(=O)c1ccccn1)C(=O)O]

To a solution of mono-fluoro sulfone (1.00 g, 2.4 mmol) in DCM (5 mL) was added TFA (5 mL). The solution was stirred at RT for 3 h and then concentrated in vacuo. The residue was re-treated under the same conditions. After concentration again the residue was dissolved in anhydrous MeOH (3 mL) and HCl in dioxane (4 M, 1 mL) was added. The solution was stirred for 15 min before being concentrated in vacuo. This was repeated twice more to yield a white powder.

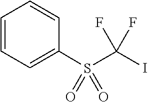 [SMILES: O=S(=O)(c1ccccc1)C(F)(F)I]

To a solution of difluoromethyl pyridyl sulfone (500 mg, 2.6 mmol) in THE (10.4 mL) at −35° C. was added iodine (2.6 g, 10.36 mmol), followed by KOtBu (1M in THF, 10.4 mL). The reaction was stirred at this temperature for 1 h at which point it was quenched with HCl (1 M, 20 mL). The aqueous phase was then extracted with EtOAc, (2×30 mL) and the combined organic layers were then washed with sat. aq. Na₂S₂O₃ (50 mL), brine (50 mL) and then dried (Na₂SO₄), filtered and concentrated in vacuo. The crude product was then purified by flash chromatography (3:7 pet. ether:DCM) to yield the desired iodo-Hu as a white solid (418 mg, 51%).

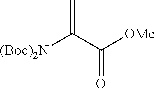

To a solution of Boc-Ser-OMe (2.68 g, 12 mmol) in MeCN (30 mL) at 0° C. was added Boc₂O (5.87 g, 26 mmol) followed by DMAP (0.30 g, 2.4 mmol). The solution was stirred gradually warming to RT over 6 h, before the addition of DBU (0.18 mL, 1.2 mmol). The mixture was stirred at RT for 16 h and then concentrated in vacuo. The residue was then dissolved in EtOAc (150 mL) and washed with HCl (1 M, 100 mL) and sat. aqueous NaHCO₃ (100 mL), then dried (Na₂SO₄), filtered and concentrated in vacuo. The crude product was then purified by flash chromatography (1:19→1:4, EtOAc:pet. ether) to yield the desired Dha as a white solid (1.80 g, 50%) AMG-2-98

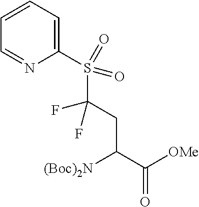

Dha (21 mg, 0.07), iodo-Hu (15 mg, 0.04), H-atom source eg Hantsch ester (15.5 mg, 0.06) and photocat (0.01 eq) were placed in a vial and ported into the glovebox. DMSO/H₂O (0.5 mL, 5:1) was then added and the vial was sealed and taken out of the glovebox, and either irradiated in the photobox or in a small multi well plate for 5 h. TLC analysis was initially used to estimate the reaction efficiency before ¹H-NMR and ¹⁹F-NMR analysis with the best reactions being around 30-40% conversion.

Bt-AA

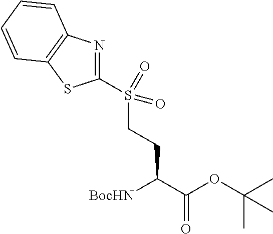 [SMILES: CC(C)(C)OC(=O)N[C@@H](CCS(=O)(=O)c1nc2ccccc2s1)C(=O)OC(C)(C)C]

To a solution of aspartic acid (4.80 g, 16.6 mmol) in THE (170 mL) at 0° C. was added iso-butyl chloroformate (6.53 mL, 49.8 mmol) followed by iPr₂NEt (4.32 mL, 24.9 mmol) and stirred at 0° C. for 3 h. NaBH₄ (4.40 g, 116.2 mmol) was added portionwise before careful addition of H₂O (38 mL) over about 30 min. The mixture was then warmed to room temperature and then diluted with EtOAc (300 mL) and washed with aqueous HCl (3×300 mL, 0.4 M), saturated aqueous NaCl (300 mL), dried (Na₂SO₄), filtered and concentrated in vacuo to yield the alcohol as a yellow oil which was used without further purification. AMG-3-43 To a solution of alcohol XX (16.6 mmol) in CH₂Cl₂ (120 mL) at 0° C. was added triethylamine (5.78 mL, 41.5 mmol) followed by slow addition of mesylchloride (1.54 mL, 19.9 mmol). The mixture was stirred at this temperature for 30 min before being poured onto saturated aqueous NaCl (200 mL). The aqueous phase was extracted with CH₂Cl₂ (3×150 mL) and the combined organic layers were dried (Na₂SO₄), filtered and concentrated in vacuo to afford the mesylate as a white solid.

To a solution of the crude mesylate in MeCN (120 mL) was added mercaptobezothiazole (3.61 g, 21.58 mmol) and triethylamine (3.47 mL, 24.9 mmol). After 16 h TLC indicated the presence of SM, and so excess K₂CO₃ (4.8 g, 34.8 mmol) was added and the reaction mixture was stirred for a further 72 h. The reaction mixture was diluted with EtOAc (250 mL) and washed with saturated aqueous NaHCO₃ (2×200 mL), water (200 mL), aqueous HCl (3×200 mL, 0.5 M) and saturated aqueous NaCl (200 mL) before being dried (Na₂SO₄), filtered and concentrated in vacuo to yield a yellow oil. AMG-3-46

To a solution crude thioether (13.42 mmol) in CH₂Cl₂ (150 mL) at 0° C. was added mCPBA (9.02 g, 40.26 mmol, 77% by weight). The mixture was stirred at this temperature for 5 h before a further portion of mCPBA (2.0 g, 8.92 mmol, 77% by weight) was added and the reaction stirred for 16 h. At this point the reaction mixture was cooled to 0° C. and quenched with 10% aqueous Na₂S₂O₃ (200 mL) and diluted with CH₂Cl₂ (200 mL). The organic phase was washed with saturated aqueous NaHCO₃ (5×400 mL), saturated aqueous NaCl (300 mL) before being dried (Na₂SO₄), filtered and concentrated in vacuo to yield the sulfone as a yellow powder. The crude product was analysed by MS and NMR. AMG-3-54

LRMS (ESI) 479.0 (M+Na⁺)

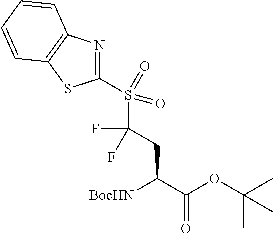 [SMILES: CC(C)(C)OC(=O)N[C@@H](CC(F)(F)S(=O)(=O)c1nc2ccccc2s1)C(=O)OC(C)(C)C]

To a solution of Bt-sulfone (410 mg, 0.90 mmol) in THE (5 mL) at −78° C. was LiHMDS (2.70 mL, 2.70 mmol, 1 M in THF) dropwise. The solution was stirred at this temperature for 5 min at which point a solution of NFSI (6.75 mL, 2.70 mmol, 0.4 M in THF) was added dropwise. The solution was stirred for 30 min at this temperature by which point TLC analysis indicated consumption of SM. The reaction mixture was then quenched at −78° C. with saturated aqueous NH₄Cl (10 mL) and Et₂O (5 mL). The aqueous layer was then extracted with Et₂O (2×15 mL) and the combined organic layers were then washed with saturated aqueous NH₄Cl (30 mL), saturated aqueous NaCl (30 mL) before being dried (Na₂SO₄), filtered and concentrated in vacuo. The crude product was then purified by flash chromatography (DCM→1:49 Et₂O:DCM) to yield the desired diF-Bt-AA as a white solid (219 mg, 49% yield from starting aspartic acid). AMG-3-55-A

LRMS (ESI) 515.0 (M+Na⁺)

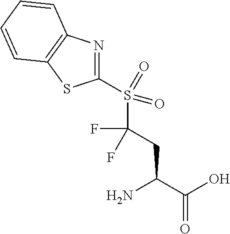 [SMILES: N[C@@H](CC(F)(F)S(=O)(=O)c1nc2ccccc2s1)C(=O)O]

To a solution of diF-Bt sulfone (180 mg, 0.37 mmol) in TFA (4.5 mL) was added water (0.5 mL). The solution was stirred at RT for 3 h and then concentrated in vacuo. The residue was dissolved in anhydrous MeOH (3 mL) and HCl in dioxane (4 M, 1 mL) was added. The solution was stirred for 15 min before being concentrated in vacuo. This was repeated twice more to yield a white powder. AMG-3-56 also AMG-3-76-cr (no decomposition) LRMS (ESI) 337.0 (M+H⁺)

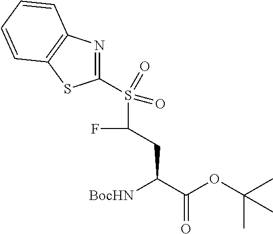 [SMILES: CC(C)(C)OC(=O)N[C@@H](CC(F)S(=O)(=O)c1nc2ccccc2s1)C(=O)OC(C)(C)C]

To a solution of Bt-sulfone (500 mg, 1.10 mmol) in THE (6 mL) at −78° C. was LiHMDS (2.20 mL, 2.20 mmol, 1 M in THF) dropwise. The solution was stirred at this temperature for 5 min at which point a solution of NFSI (3.57 mL, 1.43 mmol, 0.4 M in THF) was added dropwise. The solution was stirred for 50 min at this temperature by which point TLC analysis indicated consumption of SM. The reaction mixture was then quenched at −78° C. with saturated aqueous NH₄Cl (10 mL) and Et₂O (5 mL). The aqueous layer was then extracted with Et₂O (2×15 mL) and the combined organic layers were then washed with saturated aqueous NH₄Cl (30 mL), saturated aqueous NaCl (30 mL) before being dried (Na₂SO₄), filtered and concentrated in vacuo. The crude product was then purified by flash chromatography (DCM→1:49 Et₂O:DCM) to yield the desired monoF-Bt-AA as a white solid (258 mg, 49% yield). AMG-3-80-A

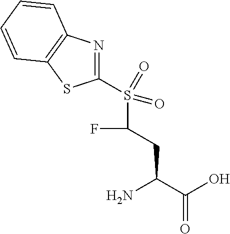 [SMILES: N[C@@H](CC(F)S(=O)(=O)c1nc2ccccc2s1)C(=O)O]

To a solution of diF-Bt sulfone (250 mg, 0.53 mmol) in TFA (4.5 mL) was added water (0.5 mL). The solution was stirred at RT for 3 h and then concentrated in vacuo. DCM was added concentrated in vacuo. This was repeated twice more to yield a white powder (210 mg, 95%). AMG-3-86

Fluoro-Lys

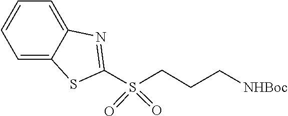 [SMILES: CC(C)(C)OC(=O)NCCCS(=O)(=O)c1nc2ccccc2s1]

To a solution of mercapto benzothiazole (2.92 g, 17.4 mmol) in MeCN (100 mL) was added K₂CO₃ (4.80 g, 34.8 mmol), NaI (350 mg, 2.33 mmol) and 3-Boc-amino-propyl bromide (5.00 g, 20.9 mmol). The reaction mixture was stirred for 16 h before being diluted with EtOAc (250 mL) and washed with water (150 mL), saturated aqueous NH₄Cl (150 mL), saturated aqueous NaCl (150 mL) before being dried (Na₂SO₄), filtered and concentrated in vacuo to yield a yellow oil.

To a solution crude thioether (17.4 mmol) in CH₂Cl₂ (200 mL) at 0° C. was added mCPBA (11.7 g, 50.2 mmol, 77% by weight). The mixture was stirred at this temperature for 5 h before a further portion of mCPBA (3.0 g, 13.38 mmol, 77% by weight) was added and the reaction stirred for 16 h. At this point the reaction mixture was cooled to 0° C. and quenched with 10% aqueous Na₂S₂O₃ (200 mL) and diluted with CH₂Cl₂ (200 mL). The organic phase was washed with saturated aqueous NaHCO₃ (5×400 mL), saturated aqueous NaCl (300 mL) before being dried (Na₂SO₄), filtered and concentrated in vacuo. The crude product was then purified by flash chromatography (2:49→1:19 EtOAc:DCM) to yield the desired Bt-sulfone as a white solid (4.85 g, 78% yield over two steps). LRMS (ESI) 379.0 (M+Na⁺)

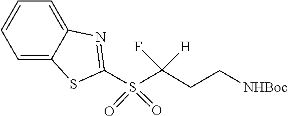 [SMILES: CC(C)(C)OC(=O)NCCC(F)S(=O)(=O)c1nc2ccccc2s1]

To a solution of sulfone (350 mg, 1.0 mmol) in THF (5 mL) at −78° C. was LiHMDS (2.0 mL, 2.0 mmol, 1 M in THF) dropwise. The solution was stirred at this temperature for 5 min at which point a solution of NFSI (3.75 mL, 1.5 mmol, 0.4 M in THF) was added dropwise. The solution was stirred for 30 min at this temperature by which point TLC analysis indicated consumption of SM. The reaction mixture was then quenched at −78° C. with saturated aqueous NH₄Cl (15 mL) and Et₂O (20 mL). The aqueous layer was then extracted with Et₂O (2×20 mL) and the combined organic layers were then washed with saturated aqueous NH₄Cl (30 mL), saturated aqueous NaCl (30 mL) before being dried (Na₂SO₄), filtered and concentrated in vacuo. The crude product was then purified by flash chromatography (DCM→3:97 Et₂O:DCM) to yield the desired monoF-sulfone as a white solid (150 mg, 40% yield). AMG-3-68A

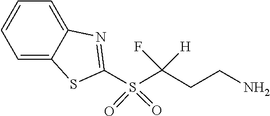 [SMILES: NCCC(F)S(=O)(=O)c1nc2ccccc2s1]

To a solution of Boc amine (73 mg, 0.195 mmol) in DCM (2 mL) was added 4M HCl in dioxane (0.48 mL, 1.95 mmol). The mixture was stirred at room temperature for 2 h and then concentrated under a stream of N₂ with co-evaporation with extra DCM added. This yielded the desired amine as the HCl salt in quantitative yield as a white solid. AMG-3-70-cr

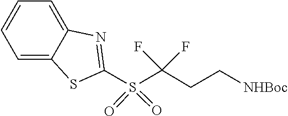 [SMILES: CC(C)(C)OC(=O)NCCC(F)(F)S(=O)(=O)c1nc2ccccc2s1]

To a solution of sulfone (350 mg, 1.0 mmol) in THF (5 mL) at −78° C. was LiHMDS (3.0 mL, 3.0 mmol, 1 M in THF) dropwise. The solution was stirred at this temperature for 5 min at which point a solution of NFSI (7.50 mL, 3.0 mmol, 0.4 M in THF) was added dropwise. The solution was stirred for 30 min at this temperature by which point TLC analysis indicated consumption of SM. The reaction mixture was then quenched at −78° C. with saturated aqueous NH₄Cl (15 mL) and Et₂O (20 mL). The aqueous layer was then extracted with Et₂O (2×20 mL) and the combined organic layers were then washed with saturated aqueous NH₄Cl (30 mL), saturated aqueous NaCl (30 mL) before being dried (Na₂SO₄), filtered and concentrated in vacuo. The crude product was then purified by flash chromatography (DCM→3:97 Et₂O:DCM) to yield the desired monoF-sulfone as a white solid (200 mg, 51% yield). AMG-3-69A

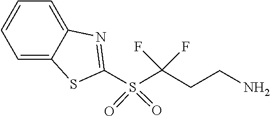 [SMILES: NCCC(F)(F)S(=O)(=O)c1nc2ccccc2s1]

To a solution of Boc amine (80 mg, 0.204 mmol) in DCM (1 mL) was added TFA (200 μL). The mixture was stirred at room temperature for 2 h and then concentrated under a stream of N₂ with co-evaporation with extra DCM added. This yielded the desired amine as the TFA salt in quantitative yield as a white solid. AMG-3-83 LRMS (ESI) (M+H⁺) 293.0

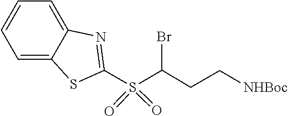 [SMILES: CC(C)(C)OC(=O)NCCC(Br)S(=O)(=O)c1nc2ccccc2s1]

To a solution of sulfone (356 mg, 1.0 mmol) in THE (5 mL) at −78° C. was LiHMDS (3.0 mL, 3.0 mmol, 1 M in THF) dropwise. The solution was stirred at this temperature for 5 min at which point a solution of NBS (531 mg, 3.0 mmol) was added. The solution was stirred for 45 min at this temperature by which point TLC analysis indicated consumption of SM. The reaction mixture was then quenched at −78° C. with saturated aqueous NH₄Cl (15 mL) and Et₂O (20 mL). The aqueous layer was then extracted with Et₂O (2×20 mL) and the combined organic layers were then washed with saturated aqueous NH₄Cl (30 mL), saturated aqueous NaCl (30 mL) before being dried (Na₂SO₄), filtered and concentrated in vacuo. The crude product was then purified by flash chromatography (DCM→3:97 EtOAc:DCM) to yield the desired monoBr-sulfone as a white solid (245 mg, 56% yield). AMG-3-84-A LRMS (ESI) (M+H⁺) 434.5, 436.5

### Example 8—Synthesis and Incorporation of pySOOF-Amino Acid into Protein

The below synthetic amino acid was incorporated into maltose binding protein using the protocol set out below, and in FIG. 7

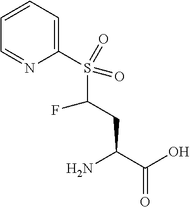 [SMILES: N[C@@H](CC(F)S(=O)(=O)c1ccccn1)C(=O)O]

according to the method of embodiment (iai) described herein.

A pyroLys tRNA, tRNA synthetase pair, and maltose binding protein (MBP) containing plasmids were co-transformed into E. coli BL21 (DE3) cells and subsequently plated out.

Single colonies were used for expression, with the amino acid dosed in at OD 0.5, followed by induction of expression by IPTG at OD=0.8 cells followed by overnight expression and harvesting of the cells.

The crude protein was then purified using Ni affinity chromatography. The desired protein containing the unnatural amino-acid was isolated with reasonable purity. Analysis of the protein via MS on Xevo confirmed the desired protein with the PySOOF AA incorporated.

### Example 9—Labelling of Proteins Using Fluoro-Bt-Sulfones

The Bt-sulfone system was used to functionalise various Dha containing proteins with a variety of substituents using the methods of embodiments (i) described herein. The reaction schemes are demonstrated in FIGS. 8A-D.

As can be seen, both diF-Bt-AA, was used to successfully label a protein, as was a biotinylated Bt-sulfone. The Bt-sulfone system was also used to generate a fluorinated lys analogue on the protein.

### Example 10—¹⁸F Labelling of Proteins Using Fluoro-Bt-Sulfones

As described in above, proteins and peptides containing ¹⁸F radiolabels may be produced using the methods described herein. A number of ¹⁸F labelled proteins were produced using the methods of embodiment (i) above.

In a first step, an additive-free halogen exchange (halex) reaction with 2-((bromofluoromethyl)thio)pyridine to introduce ¹⁸F, using [¹⁸F]KF/K₂₂₂ before subsequent oxidation to the sulfone reagent was used to provide the radical precursor compound, see FIG. 9.

The reaction of FIG. 9A was carried out using the procedure set out below.

To the full-batch of activity (3.4 GBq of dried [¹⁸F]KF/K₂₂₂), a solution of precursor (11.1 mg, 0.04 mmol in 0.5 mL MeCN) was added and the solution was left to stir at 110° C. for 10 min. The crude reaction containing the ¹⁸F-labelled compound was then allowed to cool prior to dilution with 4 mL of H₂O. The mixture was then filtered through a C18 plus cartridge (pre-conditioned with EtOH (10 mL) and H₂O (10 mL)). A solution containing NaIO₄ (52 mg, 0.24 mmol) and RuCl₃-xH₂O (2 mg, 0.010 mmol) in H₂O (4 mL) was passed through the C18 plus cartridge, pausing for 30 s after every 1 mL. After complete addition, the oxidation was left for 5 min at room temperature. The crude labelled sulfone reagent was then eluted from the cartridge with 1.2 mL of MeCN prior to semi-prep HPLC purification (55% MeCN in 25 mM ammonium formate buffer). The peak corresponding to benzothiazole sulfone CH¹⁸FF was collected at approximately 12.5 min (retention time) in a collection vial containing 20 mL of water. This solution was then passed over a C18 plus cartridge (pre-conditioned with EtOH (10 mL) and H₂O (10 mL)). The reagent was then eluted from the cartridge into a reaction vial with Et₂O (˜1.2 mL total volume). Aliquots of the Et₂O solution containing the purified ¹⁸F-sulfone reagent were dispensed into reaction vials such that the starting activity for each protein labelling reaction should be approximately 25-30 MBq. This solution was then concentrated to dryness under a flow of N₂ at rt. Protein solution containing photocatalyst, iron and DMSO under buffered conditions was then added under N₂. Histone NTEV R2Dha was functionalized with the ¹⁸F labelled BtSOOF as shown in FIG. 9A, using the reaction conditions in the table below (RCY=radiochemical yield).

| | Protein Conc. | | Equiv. of | | Equiv. | |
|---|---|---|---|---|---|---|
| No. | (mg/ml) | Photocat. | Photocat. | Additive | of Iron | RCY |
| 1 | 4 (250 μM) | Rubpy₃Cl₂•H₂O | 2 | FeSO₄•7H₂O | 250 | 53% |
| 2 | 2 (125 μM) | Rubpy₃Cl₂•H₂O | 2 | FeSO₄•7H₂O | 250 | 41% |

Formation of a desired ¹⁸F-labelled compound was confirmed by RP-HPLC, comparing the retention time with that of the cold reference product.

The reaction of FIG. 9B was carried out using the procedure below.

To the full-batch of activity (12.55 GBq of dried [¹⁸F]KF/K₂₂₂), a solution of precursor (10.4 mg, 0.04 mmol in 0.5 mL MeCN) was added and the solution was left to stir at 110° C. for 10 min. The crude reaction containing the ¹⁸F-labelled compound was then allowed to cool prior to dilution with 4 mL of H₂O. The mixture was then filtered through a C18 plus cartridge (pre-conditioned with EtOH (10 mL) and H₂O (10 mL)). A solution containing NaIO₄ (52 mg, 0.24 mmol) and RuCl₃-xH₂O (2 mg, 0.010 mmol) in H₂O (4 mL) was passed through the C18 plus cartridge, pausing for 30 s after every 1 mL. After complete addition, the oxidation was left for 5 min at room temperature. The crude labelled sulfone reagent was then eluted from the cartridge with 1.2 mL of MeCN prior to semi-prep HPLC purification (55% MeCN in 25 mM ammonium formate buffer). The peak corresponding to benzothiazole sulfone CH¹⁸FF was collected at approximately 13.5 min (retention time on Gemini column) in a collection vial containing 20 mL of water. This solution was then passed over a C18 plus cartridge (pre-conditioned with EtOH (10 mL) and H₂O (10 mL)). The reagent was then eluted from the cartridge into the corresponding reaction vial with Et₂O (˜1.2 mL total volume). This solution was then concentrated to dryness under a flow of N₂ at rt. Protein solution containing photocatalyst, iron and DMSO under buffered conditions was then added under N₂.

Histone NTEV R2Dha was functionalized with the ¹⁸F labelled mono-BtSOOF as shown in FIG. 9B, using the reaction conditions in the table below.

| | Protein Conc. | | Equiv. of | | Equiv. | |
|---|---|---|---|---|---|---|
| No. | (mg/ml) | Photocat. | Photocat. | Additive | of Iron | RCY |
| 1 | 2 (125 μM) | Rubpy₃Cl₂•H₂O | 2 | FeSO₄•7H₂O | 250 | 67% |
| 2 | 2 (125 μM) | Rubpy₃Cl₂•H₂O | 2 | FeSO₄•7H₂O | 100 | 53% |

Protein Purification

The remaining ¹⁸F-labelled protein reaction mixture was loaded onto a PD MiniTrap G-25 (pre-equilibrated with HEPES (100 mM, pH 7.4)) and then eluted with 800 μL of HEPES buffer.

MS analysis showed minimal oxidation and the expected Dha protein mass (16003 Da). The mass corresponding to background 19F-CH₂F Histone H3 is not observed due to the higher molar activity of mono-BtSOOF compared to BtSOOF.

In FIG. 9C human histone EH3 K4Dha was functionalized with the ¹⁸F labelled mono-BtSOOF, see FIG. 9C, using the reaction conditions in the table below.

| | | | Equiv. of | | Equiv. | | |
|---|---|---|---|---|---|---|---|
| No. | Protein Conc. | Photocat. | Photocat. | Additive | of Iron | Conditions | RCY |
| 4 | 2.21 mg/mL | Rubpy₃Cl₂•H₂O | 2 | FeSO₄•7H₂O | 250 | 50 W, | 57% (15 min) |
| | (125 μM) | | | | | 15 and 30 min | 58% (30 min) |
| 5 | 2.21 mg/mL | Rubpy₃Cl₂•H₂O | 0.5 | FeSO₄•7H₂O | 250 | 10 W, | 56% (30 min) |
| | (125 (μM) | | | | | 30 and 45 min | 62% (45 min) |

Good RCY was observed for the ¹⁸F-labelling of Human Histone H3 with only low levels of oxidation. As mentioned earlier, the corresponding ¹⁹F-labelled protein is not observed by MS. Milder conditions, e.g. reduced light power, were used here which reduced any double addition, which may occur with standard conditions (50 W, 15 min) when mono-BTSOOF was employed as a fluorinating reagent fot human histone eH3.

In FIG. 9D neurofilament light chain (NfL Dha) was functionalized with the ¹⁸F labelled mono-BtSOOF, see FIG. 9D, using the reaction conditions in the table below.

| | | | Equiv. of | | Equiv. | |
|---|---|---|---|---|---|---|
| No. | Protein Conc. | Photocat. | Photocat. | Additive | of Iron | RCY |
| 1 | ~70 μM | Rubpy₃Cl₂•H₂O | 5 | FeSO₄•7H₂O | 400 | 32% |

A RadioHPLC trace of the product was obtained, and showed successful labelling of NfL with good RCY (32%). This is improved compared with the RCY obtained with ¹⁸F-BtSOOF (10%). The molar activity of ¹⁸F-mono-BtSOOF is higher than ¹⁸F-BtSOOF (the difluoroalkylating reagent).

### Example 11—Biocompatability in Zebrafish

To investigate the biocompatibility of the reaction conditions zebrafish, zebrafish (3 dpf, n=25 per condition) were anaesthetized in Tricaine and injected (˜2 nL, reagents in 10 mM Tris pH 7.5) into the lower back of the head. There were 4 injection conditions:
(1) No injection control. A negative control and baseline for survival observations.
(2) Full reaction conditions minus the Dha containing protein. This was to discount potential histone toxicity if the larvae were to die as well as check background reactivity of the reagents minus a Dha substrate.
(3) Full reaction conditions. This is the full experimental condition, including both Ru(bpy)₃Cl₂ and BtSOOF-biotin (as described in the above examples).
(4) Full reaction conditions minus blue light exposure. This is to prove that light is a required trigger for the reaction and that the products didn't form spontaneously or prior to microinjection.

After microinjections, the larvae were place in a new petri dish of E3 media, where they rapidly regained mobility. For conditions requiring light exposure, the petri dish was placed directly above the 50 W blue LED in the photobox for 5 min. For all conditions, 5 of the 25 larvae were placed in a separate petri dish for survival monitoring. Not a single larvae died for 2 days following the microinjections for any condition, indicating exceptional biocompatibility of the reagents

1. A method of functionalizing a protein or peptide with a functional side chain moiety, wherein the protein or peptide comprises at least one singly occupied molecular orbital (SOMO) acceptor residue,
wherein said SOMO acceptor is a residue comprising a side chain having an alkene group;
wherein the method comprises:
(a) contacting the protein or peptide with a radical precursor compound and a photocatalyst having an oxidative half potential (Eₒₓ) of less than or equal to +1.2 V in its photo-activated state, when measured against a saturated calomel electrode and
(b) exposing the resultant composition to light radiation in order to provide a functionalized protein or peptide;
wherein the radical precursor compound is selected from formula (II) or formula (III) below 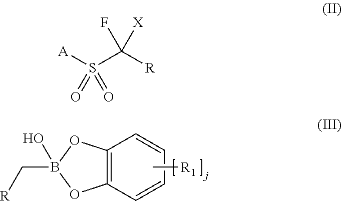
wherein R is the functional side chain moiety which is attached to the protein or peptide via the group —CFX— where the compound of formula (II) is used, or via the group —CH₂— where the compound of formula (III) is used;
X is selected from the group consisting of hydrogen, fluorine, chlorine, —C(O)OH, and —C(O)NH₂;
A is an aryl or heteroaryl group, which is optionally substituted by one or more R₂ groups;
j is 0, 1, 2, or 3;
R₁ and R₂ are independently selected from the group consisting of halogen and C₍₁₋₆₎ alkyl which is unsubstituted or substituted with one or more groups selected from hydroxy, oxy, halogen, amino, carboxy, C₍₁₋₆₎ ester, and C₍₁₋₆₎ ether; and
wherein when a compound of formula (II) is used as the radical precursor, step (a) further comprises contacting the protein or peptide with a source of Fe(II).

2. A method according to claim 1 wherein R is (i) a group selected from pharmaceutical drugs, sugars, polysaccharides, peptides, proteins, vaccines, antibodies, nucleic acids, viruses, labelling compounds, stabilized radical precursors, biomolecules and polymers, any of which may optionally be connected via a linker group.

3. A method according to claim 2, wherein the linker is a group L1 which is selected from alkyl in which one or more non-adjacent carbon atoms may be optionally substituted for a group selected from NH, O, S, —C(O)NH— or —NHC(O)—; polyethyleneglycol and analogues thereof, saccharides; polysaccharides; polyglycine; polyamides; or combinations of two or more of these groups.

4. A method according to claim 1 wherein R is (ii) a functional group R^{F}; or one or more functional groups R^{F} connected via a linker group L2; wherein R^{F} is
hydrogen, C₃₋₁₀ cycloalkyl, aryl or heteroaryl; wherein the cycloalkyl, aryl and heteroaryl groups are unsubstituted or substituted by one or more groups selected from ═O, ═NR^{a}, Y and (C₁₋₆ alkyl)-Y; or
a reactive group Y selected from C₂₋₆ alkenyl, C₂₋₆ alkynyl, halogen, hydroxy, —OR^{a}, —SR^{a}, —S(O)R^{a}, —S(O)₂R^{a}, —OSO₃R^{a}, —NR^{a}C(O)R^{b}, —NR^{a}CO₂R^{b}, —NHC(O)NR^{a}R^{b}, —NHCNH₂NR^{a}R^{b}, —NR^{a}SO₂R^{b}, —N(SO₂R^{a})₂, —NHSO₂NR^{a}R^{b}, —OC(O)R^{a}, —C(O)R^{a}, —CO₂R^{a}, —C(O)NR^{a}R^{b}, —C(O)(NHNH₂), —ONH₂, —C(O)N(OR^{a})R^{b}, —SO₂NR^{a}R^{b} or —SO(NR^{a})R^{b}; cyano, nitro, C₁₋₆ azidoalkyl, —NR^{a}R^{b} and —(NR^{a}R^{b}R^{c})⁺;
wherein:
R^{a}, R^{b}, and R^{c} independently in each instance represent hydrogen, C₁₋₆ alkyl, C₃₋₁₀ cycloalkyl, heterocyclyl, phenyl, benzyl and heteroaryl, wherein the alkyl, cycloalkyl, heterocyclyl, phenyl, benzyl and heteroaryl groups at R^{a}, R^{b}, and R^{c} are unsubstituted or substituted by one or more substituents selected from halogen, hydroxy, ═O, —NH₂, —SO₃⁻, and C₁₋₆ alkoxy; and
L2 is selected from alkyl in which one or more non-adjacent carbon atoms may be optionally substituted for a group selected from NH, O, S, —C(O)NH— or —NHC(O)—; polyethyleneglycol and analogues thereof; saccharides; polysaccharides; polyglycine; polyamides; or combinations of two or more of these groups.

5. The method of claim 1 or claim 4, wherein R is (ii) a functional group R^{F}; or one or more functional groups R^{F} connected via a linker group L2, wherein R^{F} is a reactive moiety selected from: C₂₋₆ alkenyl, C₂₋₆ alkynyl, halogen, —OC(O)R^{a}, —C(O)R^{a}, —CO₂R^{a}, —C(O)(NHNH₂), —ONH₂ and C₁₋₆ azidoalkyl; or R contains a reactive moiety of formula 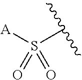
wherein A is as defined in claim 1; and
wherein the reactive moiety 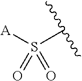
may optionally be connected via a linker group L2;
wherein L2 is an alkyl group in which one or more non-adjacent carbon atoms may be optionally substituted for a group selected from NH, O, S, —C(O)NH— or —NHC(O)—.

6. The method of claim 5 wherein the reactive moiety is selected from halogen, C₁₋₆ azido, C₂₋₆ alkynyl, 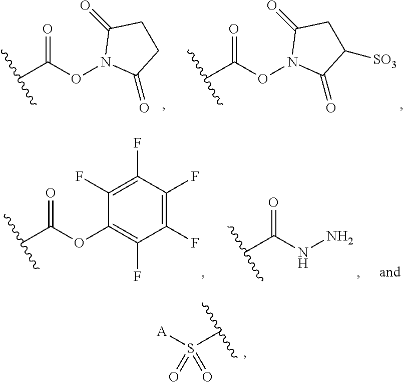
preferably

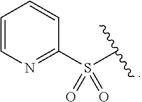 [SMILES: *S(=O)(=O)c1ccccn1]

7. A method of functionalizing a protein or peptide comprising at least one SOMO acceptor residue as defined in claim 1 with a functional side chain moiety, wherein the method comprises:
(a) contacting the protein or peptide with a radical precursor compound, a source of Fe(II) and a photocatalyst having an oxidative half potential (Eₒₓ) of less than or equal to +1.2 V in its photo-activated state when measured against a saturated calomel electrode; and
(b) exposing the resultant composition to light radiation in order to provide a functionalized protein or peptide;
wherein the radical precursor compound is a group of formula (IV) below, 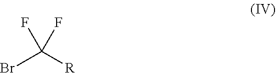
wherein R is the functional side chain moiety, which is attached to the protein or peptide via the group —CFX—; and wherein the group R is selected from —COOR^{d} and —CONR^{d}R^{e} wherein R^{d} represents hydrogen, C₁₋₆ alkyl, C₃₋₁₀ cycloalkyl, heterocyclyl, phenyl, benzyl or heteroaryl, wherein the alkyl, cycloalkyl, heterocyclyl, phenyl, benzyl, and heteroaryl groups at R^{d} are unsubstituted or substituted by one or more substituents selected from halogen, hydroxy, ═O, —NH₂, C₁₋₆ alkoxy and —NHCOR^{e}; and R^{e} represents hydrogen or C₁₋₄ alkyl.

8. A method of functionalizing a protein or peptide comprising at least one SOMO acceptor residue as defined in claim 1 with a functional side chain moiety having the structure 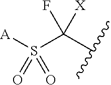
wherein the method comprises
(a) contacting the protein or peptide with a radical precursor compound, a source of Fe(II) and a photocatalyst having an oxidative half potential (Eₒₓ) of less than or equal to +1.2 V in its photo-activated state, when measured against a saturated calomel electrode; and
(b) exposing the resultant composition to light radiation in order to provide a functionalized protein or peptide;
wherein the radical precursor compound used has the following structure 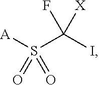
wherein the groups A and X are as defined in claim 1.

9. The method according to any one of claims 3 to 6, wherein when the functional side chain moiety comprises a reactive moiety as defined in one of claim 4 to 6, the method further comprises reacting the peptide or protein via one of the reactive moieties to connect the functional side chain to a further molecule.

10. The method according to claim 9, wherein the further molecule is a pharmaceutical drug, a sugar, a polysaccharide, a peptide, a protein, a vaccine, an antibody, a nucleic acid, a virus, a labelling compound, a biomolecule or a polymer.

11. The method according to any preceding claim wherein the SOMO acceptor residue is dehydroalanine.

12. The method according to any one of claims 1 to 6 and 8 to 11, wherein the group A is phenyl, pyridinyl, pyrimidinyl, benzothiazolyl or pyrazinyl, preferably pyridinyl, pyrimidinyl or benzothiazolyl.

13. The method according to claim 12, wherein the group A is 2-pyridinyl.

14. The method according to any one of claims 1 to 6 and 8 to 13 wherein the group X is fluorine.

15. The method according to any preceding claim, wherein the source of Fe(II) is iron(II)sulfate, FeOTf₂, Fe(ClO₄)₂, FeF₂, or (NH₄)₂Fe(SO₄)₂, preferably FeSO₄·7H₂O.

16. The method according to any preceding claim wherein the photocatalyst is a Ru(II) or Ir(II) based catalyst, preferably a Ru(II) catalyst.

17. The method according to claim 16, wherein the Ru(II) photocatalyst is Ru(bpy)₃Cl₂ or Ru(bpm)₃Cl₂.

18. The method according to any preceding claim wherein the light radiation is in the region of 300 to 600 nm, preferably 400 to 500 nm, more preferably 430 to 470 nm.

19. The method according to any one of claims 1 to 6 or 9 to 18, wherein the radical precursor compound is a compound of formula (III), and wherein the compound of formula (III) is generated in situ by contacting the protein or polypeptide in step (a) with a functionalized boron compound comprising a —BCH₂R moiety, and a catechol derivative represented by the formula (IIIB) below: 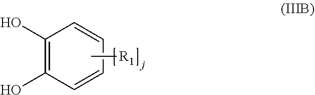
wherein R, R₁ and j are as defined in any one of claims 1 to 4.

20. A functionalized peptide or protein, comprising at least one residue of formula (IA): 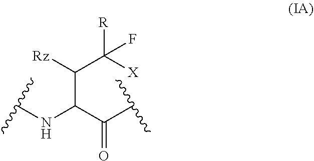
wherein X is selected from hydrogen, fluorine, —COOH, and —CONH₂, preferably fluorine;
R_{z} is hydrogen or methyl;
and
R is as defined in any one of claims 2 to 7.

21. A functionalized protein or peptide according to claim 20 wherein R is C₁₋₆ haloalkyl, C₁₋₆ azidoalkyl, or

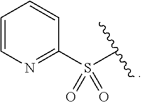 [SMILES: *S(=O)(=O)c1ccccn1]

22. A functionalized protein or peptide according to claim 20, wherein the residue of formula (IA) is any one of the compounds listed in examples 2a to 2ag.

23. A functionalized protein or peptide according to any one of claims 20 to 22 wherein X is fluorine.

24. A functionalized peptide or protein, comprising at least one residue of formula (IB): 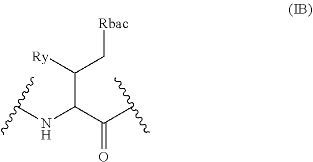
wherein Ry is hydrogen or methyl;
wherein Rbac is C₁₋₆ alkyl wherein the terminal carbon is substituted by at least one halogen, or Rbac is represented by the formula below 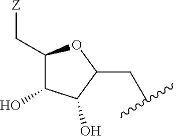
wherein Z is halogen.

25. A method of covalently linking a functionalized protein or peptide according to any one of claims 21 to 24 with a further protein or peptide, wherein the group R or Rbac in the functionalized protein or peptide is C₁₋₆ haloalkyl, and wherein the further protein or peptide comprises a group capable of reacting with an alkyl halide to form a covalent bond.

26. A method according to claim 25, wherein the functionalized protein or peptide is a substrate for the further protein or peptide, and wherein the alkyl halide group is held in a binding pocket of the other protein or peptide in order to bring said alkylhalide group into proximity with the group capable of reacting with the alkylhalide group.

27. A method of covalently linking a functionalized protein or peptide according to any one of claims 21 to 23 with a further protein or peptide, wherein the group R in the functionalized protein or peptide is 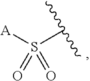
wherein the further protein or peptide comprises a group capable of reacting with a radical species to form a covalent bond, and wherein A is as defined in any one of claims 1, 12 and 13.

28. A compound according to formula (II) or (III) below: 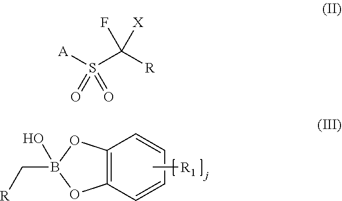
wherein A, X, R₁, and j are as defined any one of claims 1 and 12 to 14 and R is as defined in any one of claims 2 to 6.


SEQUENCE LISTING (4 sequences)

SEQ ID NO: 1 - protein, 12 aa, Artificial Sequence
  oligopeptide of page 59
  misc_feature (1)..(12): Beta amino acid
STYCHRKWDENQ

SEQ ID NO: 2 - protein, 7 aa, Artificial Sequence
  Histone NTEV fragment of Fig 9
GQFYLNE

SEQ ID NO: 3 - protein, 6 aa, Artificial Sequence
  His-tag
HHHHHH

SEQ ID NO: 4 - protein, 8 aa, Artificial Sequence
  FLAG tag
DYKDDDDK